(19) United States
(12) Patent Application Publication
(10) Pub. No.: US 2024/0327529 A1
(43) Pub. Date: Oct. 3, 2024

(54) STEROID SPARING

(71) Applicant: ASTRAZENECA AB, SODERTALJE (SE)

(72) Inventors: WENDY WHITE, GAITHERSBURG, MD (US); XIANG GUO, WILMINGTON, DE (US)

(21) Appl. No.: 18/253,280

(22) PCT Filed: Nov. 17, 2021
(86) PCT No.: PCT/EP2021/081971

§ 371 Date: May 17, 2023

Related U.S. Application Data
(60) Provisional application No. 63/115,286, filed on Nov. 18, 2020.

(51) Int. Cl. C07K 16/28; A61K 39/00; A61P 7/00; C12Q 1/6883; G01N 33/68

(52) U.S. Cl. CPC C07K 16/2866; A61P 7/00; C12Q 1/6883; G01N 33/6869; A61K 2039/505; A61K 2039/545; C07K 2317/52; C07K 2317/565; G01N 2800/52

(57) ABSTRACT

The disclosure relates to methods and compositions for the treatment of Systemic Lupus Erythematosus (SLE). Specifically, the disclosure relates to methods comprising administering to a subject a type I IFN receptor inhibitor.

### 1 BACKGROUND

A biomarker is termed as a defined characteristic that is measured as an indicator of normal biological processes, pathogenic processes or responses to an exposure or intervention. Predictive biomarkers may be used to predict response to therapy or disease course.

Systemic lupus erythematosus (SLE) is an autoimmune disease that causes significant morbidity and mortality¹. Clinical manifestations of SLE include, but are not limited to constitutional symptoms, alopecia, rashes, serositis, arthritis, nephritis, vasculitis, lymphadenopathy, splenomegaly, hemolytic anemia, cognitive dysfunction and other nervous system involvement. These disease manifestations cause a significant burden of illness and can lead to reduced physical function, loss of employment, lower health-related quality of life (QoL) and a lifespan shortened by 10 years. Increased hospitalizations and side effects of medications including chronic oral corticosteroids (OCS) and other immunosuppressive treatments add to disease burden in SLE.

Despite intense SLE clinical trial activity, only one drug, belimumab, has received regulatory approval in the last 60 years. Many factors have contributed to drug development failures in SLE, including trial design challenges, heterogeneous patient populations, and a lack of robust endpoints. Treatment of SLE is challenging because of the limited efficacy and poor tolerability of standard therapy². Many of the therapies currently used for the treatment of SLE have well known adverse effect profiles and there is a medical need to identify new targeted therapies, particularly agents that may reduce the requirement for corticosteroids and cytotoxic agents.

Most clinical trials for new treatments for SLE have failed to meet their primary and second endpoints. Part of the reason for the failure of these clinical trials may be the heterogeneity of disease manifestations in SLE patients. Furthermore, the extreme heterogeneity of the SLE has hindered the identification of biomarkers for predicting the response of SLE patients to therapy. Despite decades of investigation, there is currently no reliable biomarker for predicting the likelihood of patient response to treatment³. There is thus a need for predictive biomarkers to predict therapeutic responses in SLE patients.

The present invention solves one or more of the above-mentioned problems.

### 2 SUMMARY

The present inventors surprisingly demonstrate that high baseline IL10 was associated with worse clinical response of SLE patients and that IL10-low patients respond better to treatment than other patients. The present invention therefore provides for the first time a predictive biomarker for response to therapy in SLE patients.

The invention relates to a method of selecting a subject with SLE for treatment with a type I IFN receptor (IFNR) inhibitor, the method comprising selecting the subject for treatment if the subject's IL-10 plasma concentration is lower than a predetermined value, wherein the treatment reduces SLE disease activity in the subject; and pharmaceutical compositions of use in such a method.

The invention also relates to a method of selecting a subject with SLE for treatment with a type I IFN receptor (IFNR) inhibitor and an IL-10 inhibitor, the method comprising selecting the subject for treatment if the subject's IL-10 plasma concentration is higher than a predetermined value, wherein the treatment reduces SLE disease activity in the subject; and pharmaceutical compositions of use in such a method.

The invention further relates to a method of treating SLE in a subject in need thereof, the method comprising administering a therapeutically effective amount of an IFNR inhibitor, wherein the subject is identified as having an IL-10 plasma concentration lower than a predetermined value, wherein the treatment reduces SLE disease activity; and pharmaceutical compositions of use in such a method.

The invention further relates to a method of selecting a subject with SLE for treatment with an anti-BAFF monoclonal antibody, the method comprising selecting the subject for treatment if the subject's IL-10 plasma concentration is higher than a predetermined value, wherein the treatment reduces SLE disease activity in the subject. The invention also relates to a method of treating SLE in a subject in need thereof, the method comprising administering a therapeutically effective amount of an anti-BAFF monoclonal antibody and an anti-CD20 antibody, wherein the subject is identified as having an IL-10 plasma concentration higher than a predetermined value, wherein the treatment reduces SLE disease activity.

The invention is supported inter alia by data presented for the first time herein. Administration of anifrolumab leads to a rapid (as early as Week 8) and sustained BICLA response in SLE patients as demonstrated in placebo controlled double-blinded clinical trials (see Examples 1 to 4). The treatment effect of anifrolumab relative to placebo was consistent across preconceived subgroups (by age, gender, race, ethnicity, disease severity [SLEDAI-2K at baseline], and baseline OCS use) (see Example 5). IFNGS test-high and IL10-low patients respond better to anifrolumab treatments than other patients (Examples 6 and 7). In particular, the present invention is based on the surprising observation by the inventors that SLE patients with low levels of IL-10 respond better to treatment with a type I IFN inhibitor than SLE patients expressing high levels of IL-10. The IL-10 levels in SLE patients are correlated with the type I IFN gene signature (IFNGS) and high IL-10 is associated with more severe SLE disease.

### 3 BRIEF DESCRIPTION OF THE DRAWINGS

FIG. 1: IFN scores and efficacy of anifrolumab

FIG. 1A: IFN score distribution; FIG. 1B: Proportion of BICLA responders per visit; FIG. 1C: Kaplan-Meier curve for time to BICLA response sustained up to Week 52.

FIG. 2: Anifrolumab neutralizes 21-gene type I IFN PD signature in whole blood of SLE patients

FIG. 3: Downregulation of serum BAFF and Ficolin-3 by anifrolumab

CP1013 (NCT01438489); CP1145 (NCT01753193); ANI: anifrolumab.

FIG. 4: Long-term IL-10 and TNFα down-regulation by anifrolumab

FIG. 5: Higher SRI(4) response rate in IFNGS test-high and IL10 low patients (MUSE)

FIG. 5A: IFN-high & IL-10 low. FIG. 5B: IFN-high & IL-10 high.

FIG. 6: Higher SRI(4) response rate in IFNGS test-high and IL-10 low patients (TULIP I)

FIG. 6A: IFN-high & IL-10 high. FIG. 6A: IFN-high & IL-10 low.

FIG. 7: Higher BICLA response rate in IFNGS test-high and IL-10 low patients (MUSE)

FIG. 7A: IFN-high & IL-10 high. FIG. 7B: IFN-high & IL-10 low.

FIG. 8: Higher BICLA response rate in IFNGS test-high and IL-10 low patients (TULIP I)

FIG. 8A: IFN-high & IL-10 high. FIG. 8A: IFN-high & IL-10 low.

FIG. 9: 21-gene signature fold change vs IL-10 at Baseline (+/−GSE) (TULIP I)

FIG. 10: SLEDAI-2k total score vs IL-10 at Baseline (+/−SEM) (TULIP I)

FIG. 11: Anti-dsDNA vs IL-10 at Baseline (+/−GSE) (TULIP I)

Groups are shown in Table 14-1.

FIG. 12: IFNGS/IL-10 stratified vs anti-dsDNA (TULIP I)

Groups are shown in Table 14-1.

FIG. 13: Lymphocytes vs IL-10 baseline (+/−GSE) (TULIP I)

Groups are shown in Table 14-1.

FIG. 14: IL-10 concentration stratified by IFNGS (TULIP I)

FIG. 15: IL-10 concentration stratified by C3 levels (TULIP I)

FIG. 16: IL-10 concentration stratified by C4 levels (TULIP I)

FIG. 17: IL-10 change by IFN-test (geometric mean +/−GSE) (TULIP I)

FIG. 18: Plasma cells and auto-antibody production are primary targets of IL-10

FIG. 19: The IL-10 dependent increase of auto-AB leads to an increase of IFN1 production by dendritic cells

APC: antigen presenting cells; pDC: plasmacytoid dendritic cells; mDC: monocytic dendritic cells.

FIG. 20: The IL-10 dependent increase of auto-AB leads to an increase of cytotoxic T-cells

FIG. 21: The effect of IL-10 on Th1/Th2 responses

FIG. 22: Correlation between IL-10 and IFN1 gene21 score (MUSE)

FIG. 23. Correlation between IL-10 and SLEDAI 2K score (MUSE)

FIG. 24. Association between IL-10 and dsDNA (MUSE)

Anti-dsDNA antibody levels were classified as positive (>15 U mL 1) or negative (≤15 U mL 1) and were measured in a central laboratory using an automated fluoroimmunoassay.

FIG. 25. Association between IL-10 and autoantibodies (MUSE)

FIG. 26: Association between IL-10 and IFN-alpha (MUSE)

FIG. 27: IL-10 and lymphocyte and neutrophil levels (MUSE)

FIG. 27A: Inverse association between IL-10 and blood lymphocyte levels (MUSE). FIG. 27B: Association between IL-10 and neutrophil levels (MUSE).

FIG. 28. Synergistic effect of IL-10 and IFN1 gene score (anti-dsDNA) (MUSE)

FIG. 29. Synergistic effect of IL-10 and IFN1 gene score (SLEDAI2K score) (MUSE)

FIG. 30. Higher IL10 levels in SLE and its association with IFNGS test status (MUSE)

FIG. 30A: IFNGS test status. FIG. 30B SLEDAI score; FIG. 30C: anti-dsDNA levels; Anti-dsDNA antibody levels were classified as positive (>15 U mL⁻¹) or negative (≤15 U mL⁻¹) and were measured in a central laboratory using an automated fluoroimmunoassay. FIG. 30D: C3 levels. Complement levels were classified as abnormal (C3 <0.9 g L⁻¹; C4 <0.1 g L⁻¹) or normal (C3 ≥0.9 g L⁻¹; C4 ≥0.1 g L⁻¹) and were measured in a central laboratory. Y-axis is IL-10 serum concentration (pg/ml).

FIG. 31. IL10 is associated with complement C4 levels (MUSE)

Y-axis is serum concentration of IL-10 (pg/ml). Complement levels were classified as abnormal (C3 <0.9 g L⁻¹; C4 <0.1 g L⁻¹) or normal (C3 ≥0.9 g L⁻¹; C4 ≥0.1 g L⁻¹) and were measured in a central laboratory.

FIG. 32. Baseline IL10 level is associated with clinical response at day 365 after anifrolumab treatment (MUSE)

Y-axis is serum concentration of IL-10 (pg/ml)

FIG. 33. Baseline IL10 level is associated with clinical response at day 365 after anifrolumab treatment (MUSE)

Y-axis is serum concentration of IL-10 (pg/ml)

FIG. 34: IL10 and anifrolumab-induced clinical response in IFNGS test high patients (MUSE)

FIG. 35: Association of IL10 with clinical response in IFNGS test high patients receiving 300 mg and 1000 mg anifrolumab (MUSE)

FIG. 35A: Association of IL10 with clinical response in IFNGS test high patients receiving 300 mg and 1000 mg anifrolumab (MUSE). FIG. 35B: Comparison of IFNGS test-high & IL10-low SLE patients with other patients (MUSE); FIG. 35C: SRI(4) response status after anifrolumab treatment; FIG. 35D: Multiple regression analysis, SRI(4) response; FIG. 35E: SRI4 response without steroid tapering (MUSE).

FIG. 36: IL10 suppression by anifrolumab (MUSE)

FIG. 37: IL10 suppression by anifrolumab in IFNGS test-high patients (MUSE)

FIG. 38: Clinical responses over time in IFN-H group in IL-10 subgroups (MUSE)

FIG. 38A and FIG. 38B: Original endpoint with steroid tapering is SRI4D. FIG. 38C and FIG. 38D: new endpoint without tapering is SRI4D3

FIG. 39. Percentage of patients with a BICLA response sustained from onset to 52 in TULIP-1, TULIP-2, and pooled data from TULIP-1 and TULIP-2.

BICLA, British Isles Lupus Assessment Group-based Composite Lupus Assessment; CI, confidence interval; IFNGS, interferon gene signature; OCS, oral corticosteroid; SLEDAI-2K, Systemic Lupus Erythematosus Disease Activity Index 2000. In TULIP-1, TULIP-2, and pooled TULIP data, restricted medication rules were according to the TULIP-2 protocol. Hazard ratios and 95% CIs are estimated using a Cox regression model with treatment groups and the stratification factors (SLEDAI-2K at screening, Day 1 OCS dosage, and type I IFNGS test result at screening) as covariates.

FIG. 40. BICLA Response at All Time Points.

BICLA, British Isles Lupus Assessment Group-based Composite Lupus Assessment; IFNGS, interferon gene signature; OCS, oral corticosteroid; SLEDAI-2K, Systemic Lupus Erythematosus Disease Activity Index 2000. At early time points, P-values in TULIP-1 and TULIP-2 were 0.207 and 0.238 (Week 4), 0.020 and 0.004 (Week 8), and 0.054 and 0.029 (Week 12), respectively. In TULIP-1, TULIP-2, and pooled TULIP data, restricted medication rules were according to the TULIP-2 protocol. Responder rates are calculated using a stratified Cochran-Mantel-Haenszel approach, with stratification factors Day 1 OCS dosage, SLEDAI-2K, and type I IFNGS test result, both at screening. In the pooled analysis, an additional stratification factor is added for study. Vertical bars indicate 95% confidence intervals.

FIG. 41: Percentage of patients with a BICLA response

FIG. 41A-C illustrate the percentage of patients with a BICLA response sustained from onset to week 52 in TULIP-1 (FIG. 41A), TULIP-2 (FIG. 41B), and pooled data from TULIP-1 and TULIP-2 (FIG. 41C). Error! Reference source not found. FIG. 41D-F: illustrate the mean time to a BICLA response sustained from onset to week 52 in TULIP-1 (FIG. 41 Error! Reference source not found. D), TULIP-2 (Error! Reference source not found. FIG. 41E), and pooled data from TULIP-1 and TULIP-2 (Error! Reference source not found. FIG. 41F).

FIG. 42. MCP and PCR

FIG. 42A-C illustrate the percentage of patients achieving a major clinical response or a partial clinical response at week 24 that was sustained to week 52 in TULIP-1 (FIG. 42A), TULIP-2 (FIG. 42B), and pooled data from TULIP-1 and TULIP-2 (FIG. 42C). IFNGS, interferon gene signature; MCR, major clinical response; OCS, oral corticosteroid; PCR, partial clinical response; SLEDAI-2K, Systemic Lupus Erythematosus Disease Activity Index 2000. In TULIP-1, TULIP-2, and pooled TULIP data, restricted medication rules were according to the TULIP-2 protocol. Responder rates are calculated using a stratified Cochran-Mantel-Haenszel approach, with stratification factors Day 1 OCS dosage, SLEDAI-2K, and type I IFNGS test result, both at screening.

FIG. 43. BICLA Response by Demographic Subgroup for Pooled TULIP.

BMI, body mass index; BICLA, British Isles Lupus Assessment Group-based Composite Lupus Assessment; CI, Confidence interval; CMH, Cochran-Mantel-Haenszel. TULIP-1 data were analyzed incorporating the prespecified restricted medication rules. Differences in treatment estimates and associated 95% CIs were weighted and calculated using a stratified CMH approach.

FIG. 44: BICLA Response by SLEDAI-2K at Screening.

BICLA, British Isles Lupus Assessment Group-based Composite Lupus Assessment; CI, confidence interval; CMH, Cochran-Mantel-Haenszel; SLEDAI-2K, Systemic Lupus Erythematosus Disease Activity Index 2000. TULIP-1 data were analyzed incorporating the prespecified restricted medication rules. Differences in treatment estimates and associated 95% CIs were weighted and calculated using a stratified CMH approach.

FIG. 45. BICLA Response by Baseline Oral Corticosteroid Dosage.

BICLA, British Isles Lupus Assessment Group-based Composite Lupus Assessment; CI, confidence interval; CMH, Cochran-Mantel-Haenszel; OCS, oral corticosteroid. TULIP-1 data were analyzed incorporating the prespecified restricted medication rules. Differences in treatment estimates and associated 95% CIs were weighted and calculated using a stratified CMH approach.

FIG. 46. BICLA Response by Type I IFN Gene Signature Status (Screening)

BICLA, British Isles Lupus Assessment Group-based Composite Lupus Assessment; CI, confidence interval; CMH, Cochran-Mantel-Haenszel; IFNGS, type I interferon gene signature; qPCR, quantitative polymerase chain reaction. ^{a}Type I IFNGS was classified as either high or low by central laboratory screening using a 4-gene qPCR-based test from whole blood. TULIP-1 data were analyzed incorporating the prespecified restricted medication rules. Differences in treatment estimates and associated 95% CIs were weighted and calculated using a stratified CMH approach.

FIG. 47. Flares assessed using BILIAG-2004 in patients treated with anifrolumab compared with placebo in TULIP-2 and TULIP-1.

BILAG: British Isles Lupus Assessment Group. Note: Flare defined as 1≤ new BILAG-2004 A or 2≤ new (worsening) BILAG-2004 B domain scores as compared with the prior month's visit

FIG. 48: Time to final flare in TULIP-2 and TULIP-1

BILAG: British Isles Lupus Assessment Group. Note: Flare defined as 1≤. New BILAG-2004 A or 2≤ new (worsening) BILAG-2004 B domain scores as compared with the prior month's visit. Time to first flare is derived as data of first flare minus date of first administration of investigational product. If the patient did not have a flare, the time to flare is censored at the end of the exposure time.

FIG. 49: Annualized flare rates through week 52 in the TULIP trials.

FIG. 50: Percentage of patients with 1, 2, or three or more SLE flares through week 52 in the TULIP trials.

BILAG: British Isles Lupus Assessment Group; SLE, systemic lupus erythematosus. Flares were defined as 1≤ new BILAG-2004 A or 2≤ new BILAG-2004 B organ domain scores versus the prior visit.

FIG. 51. Percentage of Patients Achieving a CLASI-A Response by Time in Patients with SLE Receiving Anifrolumab and Placebo: TULIP-1 and TULIP-2 Pooled Data.

CLASI, Cutaneous Lupus Erythematous Disease Area and Severity Index; CLASI-A, CLASI activity score; n, number of patients in analysis; N, number of patients in treatment group; NA, not available; OCS, oral corticosteroids. A response is defined as 50%≤ reduction in CLASI activity score from baseline for patients with baseline CLASI-A 10≤. Responder rates are calculated using a stratified Cochran-Mantel-Haenszel approach, with stratification factors SLEDAI-2K score at screening. Day 1 OCS dosage, type I IFN gene signature test result at screening, and study (TULIP-1 and TULIP-2). Nominal P-values are presented, *P<0.05; **P<0.01; ***P<0.001.

FIG. 52. Time to CLASI-A Response Sustained to Week 52 in Patients with SLE Receiving Anifrolumab and Placebo: TULIP-1 and TULIP-2.

CLASI, cutaneous lupus erythematous disease area and severity index; CLASI-A, CLASI activity score; n, number of patients in analysis; N, number of patients in treatment group; NA, not available; OCS, oral corticosteroids. A response is defined as 50%≤ reduction in CLASI activity score from baseline for patients with baseline CLASI-A 10≤. Hazard ratios and 95% Cis were estimated using a Cox regression model with treatment groups with stratification (SLEDAI-2K score at screening. Day 1 OCS dosage, study, and type I IFN gene signature test result at screening) as covariates.

FIG. 53. CLASI-A Response at Week 12 by Baseline CLASI-A at 50% and 75% Response Thresholds: TULIP-1 and TULIP-2

FIG. 54: CLASI-A skin response: Example from one patient following treatment with anifrolumab (300 mg).

CLASI-A, Cutaneous Lupus Erythematosus Disease Area and Severity Index activity score. A response is defined as ≥50% reduction in CLASI-A from baseline for patients with baseline CLASI-A ≥10. In total, 13 anifrolumab-treated patients from 5 sites participated in skin photography; 2 patients had a CLASI-A response at Week 12.

FIG. 55: Flares and oral glucocorticoid use in BICLA responders vs nonresponders.

FIG. 55A: Patients with ≥1 BILAG-2004 flare through Week 52. Error bars represent 95% CI. FIG.

55B: LS mean change in oral glucocorticoid daily dosage from baseline to Week 52 in all patients regardless of baseline oral glucocorticoid dosage. Error bars represent 95% CI. FIG. 55C: Patients achieving sustained oral glucocorticoid dosage reduction to ≥7.5 mg/day among patients receiving oral glucocorticoid ≥10 mg/day at baseline. Sustained oral glucocorticoid dosage reduction defined as oral glucocorticoid dose of ≥7.5 mg/day sustained from Weeks 40 to 52. Error bars represent 95% CI. FIG. 55D: Oral glucocorticoid AUC through Week 52 for all patients regardless of baseline oral glucocorticoid dosage. Error bars represent SD. A-D, Rate difference, CIs, and nominal P values were calculated using a stratified Cochran-Mantel-Haenszel approach. AUC, area under the curve; BICLA, British Isles Lupus Assessment Group-based Composite Lupus Assessment; BILAG, British Isles Lupus Assessment Group; CI, confidence interval; LS, least squares; SD, standard deviation.

FIG. 56: PRO response at Week 52 in BICLA responders vs nonresponders.

Patients with response in (FIG. 56A) FACIT-F, defined as an improvement from baseline to Week 52>3.4; (FIG. 56B) SF-36 PCS, defined as an increase from baseline to Week 52 >3.4 in the PCS domain; and (FIG. 56C) SF-36 MCS, defined as an increase from baseline to Week 52>4.2 in the MCS domain.

FIG. 56A-C: Error bars represent 95% CI. Response rates, CIs, and nominal P values were calculated using a stratified Cochran-Mantel-Haenszel approach. FIG. 56D: LS mean change from baseline to Week 52 in PtGA score. Error bars represent 95% CI. LS mean difference, CIs, and nominal P values—calculated using mixed model repeated measures. BICLA, British Isles Lupus Assessment Group-based Composite Lupus Assessment; FACIT-F, Functional Assessment of Chronic Illness Therapy-Fatigue; MCS, mental component summary; PCS, physical component summary; PRO, patient-reported outcome; PtGA, Patient's Global Assessment; SF-36, Short Form 36 Health Survey.

FIG. 57. Change from baseline in SLEDAI-2K and PGA in BICLA responders vs nonresponders.

Change from baseline to Week 52 in (FIG. 57A) SLEDAI-2K and (FIG. 57B) PGA. Error bars represent 95% CIs. LS mean difference, CIs, and nominal P values calculated using mixed model repeated measures. BICLA, British Isles Lupus Assessment Group-based Composite Lupus Assessment; BILAG, British Isles Lupus Assessment Group; CI, confidence interval; LS, least squares; PGA, Physician's Global Assessment; SLEDAI-2K, Systemic Lupus Erythematosus Disease Activity Index 2000.

FIG. 58. CLASI-A, Joint count and organ domains.

FIG. 58A: Patients with CLASI-A response at Week 52 (defined as ≥50% reduction from baseline to Week 52) among patients with a CLASI-A score ≥10 at baseline. Response rates, CIs, and nominal P values were calculated using a stratified Cochran-Mantel-Haenszel approach. FIG. 58B, Change in LS mean joint count from baseline to Week 52 for active (defined as a joint with swelling and tenderness), tender, and swollen joints. Error bars represent 95% CIs. LS mean difference, CIs, and nominal P values calculated using mixed model repeated measures. BICLA, BILAG-based Composite Lupus Assessment; BILAG-2004, British Isles Lupus Assessment Group-2004; CI, confidence interval; CLASI-A, Cutaneous Lupus Erythematosus Disease Area and Severity Index Activity; LS, least squares: CLASI-A response and joint counts between BICLA responders and nonresponders. FIG. 58C Change in Percentages of Patients With BILAG-2004 Scores A/B and C/D/E in the Mucocutaneous and Musculoskeletal Domains From MUSE Trial Efficacy Endpoint (Week 52) to End of Follow-up (Week 60).

FIG. 59. Disease activity and Flares measures at MUSE trial efficacy endpoint (week 52) and at end of follow-up (week 60)

FIG. 59A: Disease activity; FIG. 59B: Flares.

### 4 BRIEF DESCRIPTION OF TABLES

Table 6-1: BT-063 sequences

Table 6-2:21 interferon gene signature

Table 6-3: Examples of equivalent doses of oral prednisone

Table 6-4: Anifrolumab sequences

Table 6-5: anti-IFNAR antibody sequences

Table 6-6: Anti-BAFF sequences

Table 6-7: Belimumab dosage and administration

Table 6-8: Tabalumab dosage and administration

Table 11-1: BICLA response rate at Week 52

Table 12-1: Baseline Patient Demographics

Table 12-2: Baseline Disease Characteristics

Table 13-1: Anifrolumab induced changes in serum protein levels

Table 14-1: IL-10 patient groups

Table 18-1: Patient demographics and baseline clinical characteristics

Table 18-2: Patient demographics and baseline SLE medications for BICLA responders and nonresponders

Table 18-3: SLE flares in BICLA responders and nonresponders

Table 18-4: PRO scores at baseline in BICLA responders and nonresponders

Table 18-5: Medical resource utilization for BICLA responders and nonresponders

Table 18-6: Serology changes from baseline to Week 52 for BICLA responders and nonresponders

Table 18-7: AEs during treatment in BICLA responders and nonresponders

Table 18-8: PRO scores at baseline in BICLA responders and nonresponders

### 5 DETAILED DESCRIPTION

### 5.1 Methods of Treatment and Diagnosis

The present invention in a first aspect relates to a method of selecting a subject with SLE for treatment with a type I IFN receptor (IFNR) inhibitor, the method comprising selecting the subject for treatment if the subject's IL-10 plasma concentration is lower than a predetermined value, wherein the treatment reduces SLE disease activity in the subject.

The method may comprise selecting the subject for treatment if the subject has an elevated type I interferon gene signature (IFNGS) compared to a healthy subject. The healthy subject may be a subject who does not suffer from SLE. The healthy subject may be an adult subject who does not suffer from SLE.

The elevated IFNGS may comprises at least about four-fold increase in mRNA of at least four of IFI27, IFI44, IFI44L, IFI6, and RSAD2 in a sample from the subject and/or subjects, relative to a sample from a healthy subject. The elevated IFNGS may comprise at least about four-fold increase in messenger RNA (mRNA) of at least four of IFI27, IFI44, IFI44L, IFI6, and RSAD2 in a sample from the subject and/or subjects, relative to pooled samples from healthy patients. The mRNA is increased relative to the mRNA of one or more control genes present in the sample. The one or more control genes may be chosen from ACTB, GAPDH, and 18S rRNA.

The method may comprise detecting increased mRNA of IFI27, IFI44, IFI44L, and RSAD2 in the subject. Detecting increased mRNA may comprise routine techniques in the art for measuring mRNA levels in a sample, real-time quantitative polymerase chain reaction (RT-qPCR).

The method may comprise selecting the subject for treatment if the subject is undergoing treatment comprising administration of OCS at a dose of 10 mg or more. Patients to which higher doses of OCS are administered are at greater risk of adverse events associated with OCS use.

The method may be performed in vitro. In other words, the method may be a method that is not practiced on the human or animal body.

The present invention also relates to a method of selecting a subject with SLE for treatment with a type I IFN receptor (IFNR) inhibitor and an IL-10 inhibitor, the method comprising selecting the subject for treatment if the subject's IL-10 plasma concentration is higher than a predetermined value, wherein the treatment reduces SLE disease activity in the subject. The IL-10 may compensate for the lack of response to the type I IFN inhibitor in subjects with high levels of serum IL-10 compared to the average SLE patient.

The present invention also relates to a method of treating SLE in a subject in need thereof, the method comprising administering a therapeutically effective amount of an IFNR inhibitor, wherein the subject is identified as having an IL-10 plasma concentration lower than a predetermined value, wherein the treatment reduces SLE disease activity. The subject treated by the method may be identified as having an elevated IFNGS compared to a healthy subject. The elevated IFNGS in the subject treated by the method may comprise at least about four-fold increase in mRNA of at least four of IFI27, IFI44, IFI44L, IFI6, and RSAD2 in a sample from the subject, relative to a sample from a healthy subject. The elevated IFNGS may comprise at least about four-fold increase in mRNA of at least four of IFI27, IFI44, IFI44L, IFI6, and RSAD2 in a sample from the subject, relative to pooled samples from healthy patients. The mRNA may be increased relative to the mRNA of one or more control genes present in the sample. The one or more control genes may be chosen from ACTB, GAPDH, and 18S rRNA. The method may comprise detecting increased mRNA of IFI27, IFI44, IFI44L, and RSAD2 in the subject. The subject treated by the method may be undergoing treatment comprising administration of OCS at a dose of 10 mg or more pre-treatment with the IFNR inhibitor.

The present invention also relates to a method of treating SLE in a subject in need thereof, the method comprising administering a therapeutically effective amount of an IFNR inhibitor and an IL-10 inhibitor, wherein the subject is identified as having an IL-10 plasma concentration higher than a predetermined value, wherein the treatment reduces SLE disease activity.

The present invention also relates to a method of selecting a subject with SLE for treatment with an anti-BAFF monoclonal antibody, the method comprising selecting the subject for treatment if the subject's IL-10 plasma concentration is higher than a predetermined value, wherein the treatment reduces SLE disease activity in the subject. The anti-BAFF antibody may be belimumab or a functional variant thereof. The present invention therefore also relates to a method for using a predictive biomarker for response to combined belimumab and an anti-IL-10 antibody.

The present invention also relates to a method of treating SLE in a subject in need thereof, the method comprising administering a therapeutically effective amount of an anti-BAFF monoclonal antibody and an anti-CD20 antibody, wherein the subject is identified as having an IL-10 plasma concentration higher than a predetermined value, wherein the treatment reduces SLE disease activity. The anti-CD20 antibody may be rituximab and the anti-BAFF antibody may be belimumab. The present invention therefore also relates to a method for using a predictive biomarker for response to combined belimumab and rituximab.

The methods of the invention may also comprise determining the IL-10 concentration in a sample from the patient. The sample may be any sample taken for the body that can be used to assess serum levels of IL-10. In particular, the sample may be a blood, serum or plasma sample. In order to determine the IL-10 levels in the serum of the subject, the IL-10 concentration in the sample may be determined by enzyme-linked immunosorbent assay (ELISA) or any other technique known in the art.

The predetermined value may be about 1 to about 3.5 pg/ml. The predetermined value may be about 1.5 to about 2.5 pg/ml. The predetermined value may be about 1.7 to 2.3 pg/ml. The predetermined value may be about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 1, about 2 or about 3. The predetermined value may be any of 0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 1, 2 or 3 pg/ml.

The predetermined value may particularly be about 2 pg/ml. The predetermined value may particularly be 2 pg/ml. The predetermined value may be determined by: a) determining the IL-10 plasma concentrations of subjects in a sample population of subjects with SLE; b) determining the median IL-10 concentration in the population of subjects with SLE, wherein the predetermined value is the median determined in b).

The method of any preceding claim, wherein the subject is administered steroids at a dose of 10 mg or more before treatment.

Reducing SLE disease activity in the subject may comprise one or more of any of the following:
a BILAG-Based Composite Lupus Assessment (BICLA) response in the subject,
an SRI(4) response in the subject,
reducing the subject's Cutaneous Lupus Erythematosus Disease Area and Severity Index (CLASI) score compared to the subject's CLASI score pre-treatment,
reducing the subject's tender and swollen joint count compared to the subject's tender and swollen joint count pre-treatment,
the subject having a maximum of 1 BILAG-2004 B score following treatment,
the subject having a BILAG-2004 score of C or better following treatment,
the subject having an improvement in at least one patient reported outcome (PRO) compared to pre-treatment, and
reducing the subject's SLE flare rate compared to the subject's flare rate pre-treatment.

The methods of the invention may comprise measuring the subject's BILAG score before and after administration of the IFNAR inhibitor. The BICLA response may be sustained in the subject for at least 52 weeks. The method may comprise measuring PROs in the subject before and after administration of the IFNAR inhibitor. The PRO's may comprise the subject's Functional Assessment of Chronic Illness Therapy-Fatigue (FACIT-F), Short Form 36 Health Survey version 2 (SF-36-v2), mental component summary (MCS), and/or SF-36, physical component summary (PCS) score.

The BICLA response may comprise reduction of the subject's BILAG-2004 A and B domain scores to B/C/D and C/D, respectively. Reducing the subject's CLASI score compared to the subject's CLASI score pre-treatment may comprise a reduction in the subject's CLASI-A score compared to the subject's CLASI-A score pre-treatment. Reducing the SLE disease activity in the subject may comprise reducing the anti-dsDNA levels in the subject. Reducing the SLE disease activity in the subject may comprise a BILAG-Based Composite Lupus Assessment (BICLA) response, wherein the method also comprises reducing the OCS dose administered to the subject compared to the OCS dose administered to the subject pre-treatment

The OCS comprises prednisone, prednisolone and/or methylprednisolone.

Reducing SLE disease activity in the subject may comprise a BILAG-Based Composite Lupus Assessment (BICLA) response by at least week 4 of treatment.

Reducing SLE disease activity may comprises a BILAG-Based Composite Lupus Assessment (BICLA) response by at least week 8 of treatment. Reducing SLE disease activity in the subject may comprise at least a 50% improvement in the tender joint count and swollen joint count in the subject compared to the tender joint and swollen count in the subject pre-treatment value. The reduction in the subject's CLASI score may be achieved by at least week 8 of treatment. Reduction in the subject's CLASI score may be achieved following 12 weeks of treatment. Reducing SLE disease activity in the subject may comprise at least 50% reduction in the subject's CLASI score compared to the subject's CLASI score pre-treatment. Reducing SLE disease activity in the subject may comprise reduction of the subject's CLASI-A score following 12 weeks of treatment. The subject may have a CLASI-A score of ≥10 pre-treatment. Reducing SLE disease activity in the subject may comprise the subject's BILAG-2004 score being C or better after 24 weeks of treatment. Reducing SLE disease activity in the subject may comprise the subject having a maximum of 1 BILAG-2004 B score after 24 weeks of treatment. Reducing SLE disease activity in the subject may comprise a reduction in the subject's BILAG-based annualized flare rate compared to the subject's BILAG-based annualized flare rate pre-treatment. Reducing SLE disease activity in the subject may comprise preventing flares in the subject.

A flare may be defined as ≥1 new BILAG-2004 A or ≥2 new (worsening) BILAG-2004 B domain scores compared to the subject's scores one month previously. Reducing SLE disease activity in the subject may comprise a reduced flare rate in the subject compared to the flare rate pre-treatment, wherein the method comprises reducing OCS dose administration to the subject compared to the OCS dose administered to the subject pre-treatment. The method may comprise selecting the subject for treatment, wherein the subject is selected for having active SLE. The subject may be selected for having moderate to severe SLE. The subject may be selected for having SLE that is unresponsive to OCS treatment.

The subject may be an adult.

### 5.2 Type I IFN Receptor Inhibitor

The type I IFN receptor inhibitor (IFNR, IFNAR, IFNAR1) may be administered intravenously or subcutaneously. The type I IFN receptor inhibitor may be an anti-type I interferon receptor antibody or antigen binding fragment thereof that specifically binds IFNAR1. The antibody may be a monoclonal antibody. The antibody may be anifrolumab.

The IFN receptor inhibitor may comprise a heavy chain variable region complementarity determining region 1 (HCDR1) comprising the amino acid sequence of SEQ ID NO: 3; a heavy chain variable region complementarity determining region 2 (HCDR2) comprising the amino acid sequence of SEQ ID NO: 4; a heavy chain variable region complementarity determining region 3 (HCDR3) comprising the amino acid sequence of SEQ ID NO: 5; a light chain variable region complementarity determining region 1 (LCDR1) comprising the amino acid sequence SEQ ID NO: 6; a light chain variable region complementarity determining region 2 (LCDR2) comprising the amino acid sequence SEQ ID NO: 7; and/or a light chain variable region complementarity determining region 3 (LCDR3) comprising the amino acid sequence SEQ ID NO: 8.

The IFN receptor inhibitor may comprise (a) a human heavy chain variable region comprising the amino acid sequence of SEQ ID NO: 1; and (b) a human light chain variable region comprising the amino acid sequence of SEQ ID NO: 2. The IFN receptor inhibitor may comprise an Fc region comprising an amino acid substitution of L234F, as numbered by the EU index as set forth in Kabat, and wherein the antibody exhibits reduced affinity for at least one Fc ligand compared to an unmodified antibody, optionally wherein the antibody comprises in the Fc region an amino acid substitution of L234F, L235E and/or P331S, as numbered by the EU index as set forth in Kabat. The IFN receptor inhibitor may comprise (a) a human chain comprising the amino acid sequence of SEQ ID NO: 11; and (b) a human light chain comprising the amino acid sequence of SEQ ID NO: 12.

The type I IFN receptor inhibitor may comprise sifalimumab.

The method may comprise administering an anti-IL-10 antibody to the subject. The anti-IL-10 antibody may comprise (a) a heavy chain variable region comprising the amino acid sequence of SEQ ID NO: 18; and (b) a light chain variable region comprising the amino acid sequence of SEQ ID NO: 17.

The anti-IL-10 antibody may comprise a heavy chain variable region complementarity determining region 1 (HCDR1) comprising the amino acid sequence of SEQ ID NO: 22; a heavy chain variable region complementarity determining region 2 (HCDR2) comprising the amino acid sequence of SEQ ID NO: 23; a heavy chain variable region complementarity determining region 3 (HCDR3) comprising the amino acid sequence of SEQ ID NO: 24; a light chain variable region complementarity determining region 1 (LCDR1) comprising the amino acid sequence SEQ ID NO: 19; a light chain variable region complementarity determining region 2 (LCDR2) comprising the amino acid sequence SEQ ID NO: 20; and/or a light chain variable region complementarity determining region 3 (LCDR3) comprising the amino acid sequence SEQ ID NO: 21. The IL-10 antibody may be BT-063 or a functional equivalent thereof.

The method may comprise administering anifrolumab. The treatment may comprise administering 300 mg anifrolumab. Anifrolumab may be administered as an intravenous (IV) infusion. Anifrolumab may be administered every four weeks. Anifrolumab may be provided in a solution at a concentration of 150 mg/mL.

The method may comprise administrating a type I IFN receptor inhibitor and an IL-10 inhibitor.

The present invention also relates to a pharmaceutical composition for use in a method of treating SLE in a subject in need thereof, the method of treatment comprises administering a therapeutically effective amount of an IFNR inhibitor, wherein the subject is identified as having an IL-10 plasma concentration lower than a predetermined value, wherein the treatment reduces SLE disease activity. The IFNR inhibitor may be anifrolumab. The pharmaceutical composition may comprise anifrolumab at a concentration of 150 mg/mL. The pharmaceutical composition may comprise 150 mg/mL anifrolumab; 50 mM lysine HCl; 130 mM trehalose dihydrate; 0.05% polysorbate 80; 25 mM histidine/histidine HCl, wherein the pharmaceutical composition is at a pH of 5.9.

### 5.3 Doses

The method may comprise administering an intravenous dose of anifrolumab or the functional variant thereof to the subject. The intravenous dose may be ≥300 mg anifrolumab or the functional variant thereof. The intravenous dose may be ≤1000 mg. The intravenous dose may be about 300 mg, about 900 mg or about 1000 mg. The intravenous dose may be administered every four weeks (Q4W).

The method may comprise administering a subcutaneous dose of anifrolumab or the functional variant thereof. The subcutaneous dose may be >105 mg and <150 mg anifrolumab or the functional variant thereof. The subcutaneous dose may be ≤135 mg anifrolumab or the functional variant thereof. The subcutaneous dose may be about 120 mg. The subcutaneous dose may be administered in a single administration step. The subcutaneous dose may be administered at intervals of 6-8 days. The subcutaneous dose may be administered once per week. The subcutaneous dose may have a volume of about 0.5 to about 1 ml. The subcutaneous dose may have a volume of about 0.8 ml.

### 5.4 Kit

The invention also relate to a kit for use in any method of the invention. The kit may comprise the pharmaceutical composition of the invention. The kit may comprise instructions for use. The instructions for use may specify any method of the invention. The instructions for use may specify a method comprising selecting a subject with SLE for treatment with a type I IFN receptor (IFNR) inhibitor, the method comprising selecting the subject for treatment if the subject's IL-10 plasma concentration is lower than a predetermined value, wherein the treatment reduces SLE disease activity in the subject. The instructions for use may specify a method of selecting subject with SLE for treatment with a type I IFN receptor (IFNR) inhibitor and an IL-10 inhibitor, the method comprising selecting the subject for treatment if the subject's IL-10 plasma concentration is higher than a predetermined value, wherein the treatment reduces SLE disease activity in the subject. The instruction for use may specify a method of treating SLE in a subject in need thereof, the method comprising administering a therapeutically effective amount of an IFNR inhibitor and an IL-10 inhibitor, wherein the subject is identified as having an IL-10 plasma concentration higher than a predetermined value, wherein the treatment SLE disease activity. The instructions for use may specify a method of treating SLE in a subject in need thereof, the method comprising administering a therapeutically effective amount of an anti-BAFF monoclonal antibody and an anti-CD20 antibody, wherein the subject is identified as having an IL-10 plasma concentration higher than a predetermined value, wherein the treatment reduces SLE disease activity.

The kit may comprise anifrolumab or a functional equivalent thereof. The kit may comprise an anti-IL-10 antibody. The kit may comprise belimumab or a functional equivalent thereof.

### 6 DEFINITIONS

### 6.1 Interleukin-10 (IL-10)

IL-10 (also known as Cytokine Synthesis Inhibitory Factor (CSIF), T-Cell Growth Inhibitory Factor (TGIF); UniProtKB P22301) is a predominantly anti-inflammatory cytokine that inhibits T cell function by suppressing the expression of proinflammatory cytokines such as TNFα, IL-1 IL-1B, IL-6, IL-8, IL-10, granulocyte macrophage colony-stimulating factor (GM-CSF) and IL-12. IL-10 also reduces antigen presentation by monocytes. However, in addition to its anti-inflammatory role, IL-10 also promotes B-cell survival, proliferation, differentiation, and antibody production⁴.

In a small, open-label clinical trial, anti-IL-10 treatment (anti-IL-10 murine mAb (B-N10)) of lupus patients with cutaneous and joint manifestations resulted in improvement to clinically inactive disease in five of six patients within 6 months of the 3-week treatment regimen⁵.

### 6.2 Anti-IL-10 Antibodies

SCH708980 is an anti-IL-10 monoclonal antibody investigated for the treatment of Visceral Leishmaniasis (NCT01437020). Anit-IL-10 monoclonal antibodies for the treatment of SLE are described in WO2005047326 and WO 2011.064399.

BT-063 is an anti-IL-10 antibody. BT-063 is described in WO 2011064399, which is incorporated herein by reference. The sequences of BT-063 are shown in Table 6-1.

TABLE 6-1
BT-063 sequences

| | |
|---|---|
| VL | DVVMTQSPLS LPVTLGQPAS ISCRSSQNIV |
| | HSNGNTYLEW YLQRPGQSPR LLIY KVSNRF |
| | SGVPDRFSGS GSGTDFTL I SRVEAEDVGV YYCF |
| | QGSHVP WTFGQGTKVE IK |
| | (SEQ ID No: 17) |
| VH | EVQLVESGGG LVQPGGSLRL SCA ASGFSFA TY |
| | GVHWVRQS PGKGLEWLGV IWRGGSTDYS |
| | AAFMSRLTIS KDNSKNTVYL QMNSLRAEDT |
| | AVYFCAK QAY GHYMDYWG QG TSVTVSS |
| | (SEQ ID NO: 18) |
| LCDR1 | ISCRSSQNIVHSNGNTY |
| | (SEQ ID NO: 19) |
| LCDR2 | KVSNRFSGVPDR |
| | (SEQ ID NO: 20) |
| LCDR3 | QGSHVPW |
| | (SEQ ID NO: 21) |
| HCDR1 | ASGFSFATY |
| | (SEQ ID NO: 22) |
| HCDR2 | IWRGGSTDYSAAFMSR |
| | (SEQ ID NO: 23) |
| HCDR3 | QAYGHYMD |
| | (SEQ ID NO: 24) |

### 6.3 Type I IFN Gene Signature (IFNGS)

Type I interferon (IFN) signalling drives pathology in a number of autoimmune diseases, in particular in systemic lupus erythematosus (SLE), and can be tracked via type I IFN-inducible transcripts present in whole blood—said transcripts provide a type I IFN gene signature. By way of example, Yao et al. (Hum Genomics Proteomics 2009, pii: 374312)⁶ describe the identification of an IFNα/β 21-gene signature and its use as a biomarker of type I IFN-related diseases or disorders.

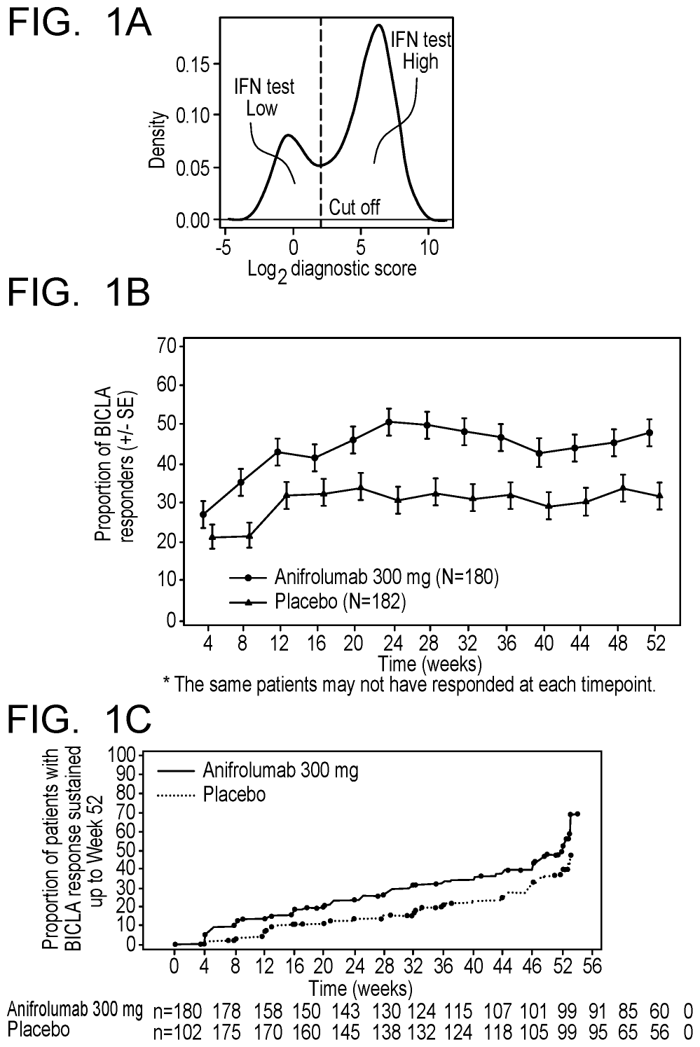

Type I IFN has been considered to be important in SLE disease pathogenesis and inhibition of this pathway is targeted by anifrolumab. To understand the relationship between type I IFN expression and response to anti-IFN therapy, it is necessary to know if a subject's disease is driven by type I IFN activation. However, direct measurement of the target protein remains a challenge. As such, a transcript-based marker was developed to evaluate the effect of over expression of the target protein on a specific set of mRNA markers. The expression of these markers is easily detected in whole blood and demonstrates a correlation with expression in diseased tissue such as skin in SLE. The bimodal distribution of the transcript scores for SLE subjects supports defining an IFN test high and low subpopulation (FIG. 1)

An IFN gene signature (IFNGS) can thus be used to identify patients with low or high levels of IFN inducible gene expression. In some embodiments, the IFNGS comprises Interferon Alpha Inducible Protein 27 (IFI27), Interferon Induced Protein 44 (IFI44) interferon induced protein 44 like (IFI44L), and Radical S-Adenosyl Methionine Domain Containing 2 (RSAD2). Up regulation or overexpression of the genes comprising the IFNGS can be calculated by well-known methods in the art. For example, the overexpression of the signature is calculated as the difference between the mean Ct (cycle threshold) for IFI27, IFI44, IFI44L, and RSAD2 and the mean Ct of three control genes; 18S, ACTB and GAPDH. The degree of increased expression of the IFNGS permits the identification of a fold change cutoff for identifying IFN-high and IFN-low patients. In one embodiment, the cutoff is at least about 2. In another embodiment, the cutoff is at least about 2.5. In another embodiment, the cutoff is at least about 3. In another embodiment, the cutoff is at least about 3.5. In another embodiment, the cutoff is at least about 4. In another embodiment, the cutoff is at least about 4.5. In another embodiment, the cutoff is chosen from at least 3.5, 3.6, 3.7, 3.8, 3.9, 4.0, 4.1, 4.2, 4.3, 4.4, and 4.5. In another embodiment the cutoff is between about 2 and about 8. The degree of increased expression of the IFNGS also permits the identification of a delta Ct cutoff for identifying IFN-high and IFN-low subpopulations.

The type I IFN gene signature (IFNGS) is described in WO 2011/028933, which is incorporated herein by reference in its entirety.

### 6.4 Genes in the Type I IFN Gene Signature

The group of genes included in the type I IFN gene signature (also referred to herein as the type I IFN or IFNa-inducible PD marker expression profile) of the patient are (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

In a specific embodiment, the group of genes included in the type I IFN or IFNa-inducible PD marker expression profile of the patient comprises IFI27, IFI44, IFI44L, IFI6 and RSAD2. In another specific embodiment, the group of genes included in the type I IFN or IFNa-inducible PD marker expression profile of the patient consists of IFI27, IFI44, IFI44L, IFI6 and RSAD2. In a further specific embodiment, the group of genes included in the type I IFN or IFNa-inducible PD marker expression profile of the patient comprises IFI27, IFI44, IFI44L, and RSAD2. In another specific embodiment, the group of genes included in the type I IFN or IFNa-inducible PD marker expression profile of the patient consists of IFI27, IFI44, IFI44L, and RSAD2.

The IFNa-inducible PD markers in an expression profile may include (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

The IFNa-inducible PD markers in an expression profile may consist of (a) IFI27, IFI44, IFI44L, IFI6 and RSAD2; or (b) IFI44, IFI44L, IFI6 and RSAD2; or (c) IFI27, IFI44L, IFI6 and RSAD2; or (d) IFI27, IFI44, IFI6 and RSAD2; or (e) IFI27, IFI44, IFI44L, and RSAD2; or (f) IFI27, IFI44, IFI44L, and IFI6.

Suitable primers and probes for detection of the genes may be found in WO2011028933, which is incorporated herein by reference in its entirety.

The IFN 21-gene signature (IFNGS) is a validated pharmacodynamic marker of type I IFN signaling¹⁰, that is elevated in patients with type I IFN-mediated disease, including SLE, lupus nephritis, myositis, Sjogren's and scleroderma.

A 4-gene IFNGS score is calculated by measurement of IFI27, IFI44, IFI44L, and RSAD2 expression. A 5-gene IFNGS score is calculated by measurement of IFI27, RSAD2, IFI44, IFI44L, IFI6 expression. A 21-gene IFNGS score is calculated by measurement of the genes shown in Table 6-2. Gene expression may be measured by detecting mRNA in the whole blood or tissue of the subject. A IFNGS (4-gene, 5-gene or 21-gene) score may be detected in a subject by measuring the IFNGS gene expression (e.g. mRNA) in the blood or tissue of the subject and comparing the gene expression levels to expression of house-keeping or control genes, e.g. ACTB, GAPDH, and 18S rRNA, in the blood or tissue.

TABLE 6-2
21 interferon gene signature

| | Gene | Gene |
|---|---|---|
| Gene title | symbol | Probe ID |
| Interferon, alpha-inducible protein 27 | IFI27 | 202411 |
| Interferon, alpha-inducible protein 6 | IF16 | 204415 |
| Radical S-adenosyl methionine domain | RSAD2 | 213797 |
| containing 2 | | |
| Interferon-induced protein 44 | IFI44 | 214059 |
| Interferon-induced protein 44-like | IFI44L | 204439 |
| Ubiquitin specific peptidase 18 | USP18 | 219211 |
| Lymphocyte antigen 6 complex, | LY6E | 202145 |
| locus E | | |
| 2,5-oligoadenylate synthetase 1, | OAS1 | 202869 |
| 40/46 kDa | | |
| Sialic acid binding Ig-like lectin 1, | SIGLEC1 | 44673 |
| sialoadhesin | | |
| ISG15 ubiquitin-like modifier | ISG15 | 205483 |
| Interferon-induced protein with | IFIT1 | 203153 |
| tetratricopeptide repeats 1 | | |
| 2′-5′-oligoadenylate synthetase | OAS3 | 218400 |
| 3, 100 kDa | | |
| Hect domain and RLD 5 | HERC5 | 219863 |
| Myxovirus (influenza virus) resistance 1 | MX1 | 202086 |
| Lysosomal-associated membrane protein 3 | LAMP3 | 205569 |
| Epithelial stromal interaction 1 (breast) | EPSTI1 | 227609 |
| Interferon-induced protein with | IFIT3 | 204747 |
| tetratricopeptide repeats 3 | | |
| 2′-5′-oligoadenylate synthetase 2, | OAS2 | 204972 |
| 69/71 kDa | | |
| Receptor (chemosensory) transporter | RTP4 | 219684 |
| protein 4 | | |
| Phospholipid scramblase 1 | PLSCR1 | 241916 |
| DNA polymerase-transactivated protein 6 | DNAPTP6 | 241812 |

### 6.5 Upregulation

The upregulation or downregulation of the type I IFN or IFNa-inducible PD markers in the patient's expression profile may be by any degree relative to that of a sample from a control (which may be from a sample that is not disease tissue of the patient (e.g., non-lesional skin of a psoriasis patient) or from a healthy person not afflicted with the disease or disorder) or may be relative to that of genes from the patient whose expression is not changed by the disease (so called “house keeping” genes.)

The degree upregulation or downregulation may be at least 10%, at least 15%, at least 20%, at least 25%, at least 30%, at least 40%, at least 50%, at least 60%, at least 70%, at least 75%, at least 80%, at least 85, at least 90%, at least 95%, at least 100%, at least 125%, at least 150%, or at least 200%, or at least 300%, or at least 400%, or at least 500% or more that of the control or control sample.

Type I IFN or IFNa-inducible PD marker expression profile may be calculated as the average fold increase in the expression or activity of the set of genes comprised by the PD marker. The Type I IFN or IFNa-inducible PD marker expression profile may also be calculated as the difference between the mean Ct (cycle threshold) for the four target genes and the mean Ct of three control genes.

The average fold increase in the expression or activity of the set of genes may be between at least about 2 and at least about 15, between at least about 2 and at least about 10, or between at least about 2 and at least about 5. The average fold increase in the expression or activity of the set of genes may be at least about 2, at least about 2.5, at least about 3, at least about 3.5, at least about 4, at least about 4.5, at least about 5, at least about 5.5, at least about 6, at least about 6.5, at least about 7, at least about 8, at least about 9 or at least about 10. [0047] The degree of increased expression permits the identification of a fold change cutoff for identifying signature positive and signature negative patients suffering from autoimmune diseases. In one embodiment, the cutoff is at least about 2. In another embodiment, the cutoff is at least about 2.5. In another embodiment, the cutoff is at least about 3. In another embodiment, the cutoff is at least about 3.5. In another embodiment, the cutoff is at least about 4. In another embodiment, the cutoff is at least about 4.5. In another embodiment, the cutoff is chosen from at least 3.5, 3.6, 3.7, 3.8, 3.9, 4.0, 4.1, 4.2, 4.3, 4.4, and 4.5. In another embodiment the cutoff is between about 2 and about 8. In one embodiment, the cutoff is the mean of the increased expression levels of at least four of IFI27, IFI44, IFI44L, IFI6 and RSAD2. In another embodiment, the cutoff is the median of the increased expression levels of at least four of IFI27, IFI44, IFI44L, IFI6 and RSAD2.

The degree of increased expression also permits the identification of a delta Ct cutoff for identifying signature positive and signature negative patients suffering from autoimmune diseases. In one embodiment, the cutoff is at least about 7.6. In another embodiment, the cutoff is 7.56. The fold change cutoff may be used to determine an appropriate delta Ct cutoff (e.g., 1<log2 of the fold change <3 corresponds to delta Ct range of 8.65 to 6.56.). Thus, in another embodiment, the delta Ct cutoff is between about 6.56 to about 8.56.

Furthermore, the patient may overexpress or have a tissue that overexpresses a type I IFN subtype at least 10%, at least 15%, at least 20%, at least 25%, at least 30%, at least 40%, at least 50%, at least 60%, at least 70%, at least 75%, at least 80%, at least 90%, at least 100%, at least 125%, at least 150%, or at least 200%, or at least 300%, or at least 400%, or at least 500% that of the control. The type I IFN subtype may be any one of IFNaI, IFNa2, IFNa4, IFNa5, IFNa6, IFNa7, IFNa8, IFNaIO, IFNaI4, IFNaI7, IFNa21, IFNp, or IFNco. The type I IFN subtypes may include all of IFNaI, IFNa2, IFNa8, and IFNaI4.

The up-regulated expression or activity of any gene detected in a sample, by probes, or by probes in kits in an IFNa-inducible PD marker expression profile may be at least 1.2-fold, at least 1.25-fold, at least 1.3-fold, at least 1.4-fold, at least 1.5-fold, at least 2.0-fold, at least 2.25-fold, at least 2.5-fold, at least 2.75-fold, at least 3.0-fold, at least 3.5-fold, at least 4.0-fold, at least 4.5-fold, at least 5.0-fold, at least 6.0-fold, at least 7.0-fold, at least 8.0-fold, at least 9.0-fold, at least 10.0-fold, at least 15.0-fold, at least 20.0-fold, at least 25.0-fold, or at least 50.0-fold relative to baseline levels of control cells, e.g., cells of healthy volunteers or cells of control animals or cells not exposed to IFNa in culture. All of the genes in the IFNa-inducible PD marker expression profile may have up-regulated expression or activity at the same fold increase. Alternatively, the genes in the PD marker expression profile may have varying levels of up-regulated expression or activity.

### 6.6 Measuring Upregulation

Up-or down-regulation of gene expression or activity of IFNa-inducible PD markers may be determined by any means known in the art. For example, up-or down-regulation of gene expression may be detected by determining mRNA levels. mRNA expression may be determined by northern blotting, slot blotting, quantitative reverse transcriptase polymerase chain reaction, or gene chip hybridization techniques. See U.S. Pat. Nos. 5,744,305 and 5,143,854 for examples of making nucleic acid arrays for gene chip hybridization techniques. The TAQMAN® method may be used for measuring gene expression^{7,8}.

Primers that selectively bind to targets in polymerase chain reactions (PCR) can be chosen based on empirically determining primers that hybridize in a PCR reaction and produce sufficient signal to detect the target over background, or can be predicted using the melting temperature of the primentarget duplex as described in Maniatis et al. Molecular Cloning, Second Edition, Section 11.46. 1989. Similarly, probes for detecting PCR products in a TAQMAN® or related method can be empirically chosen or predicted. Such primers and probes (collectively “oligonucleotides”) may be between 10 and 30 nucleotides or greater in length.

Up- or down-regulation of gene expression or activity of IFNa-inducible PD markers may be determined by detecting protein levels. Methods for detecting protein expression levels include immuno-based assays such as enzyme-linked immunosorbant assays, western blotting, protein arrays, and silver staining. An IFNa-inducible PD marker expression profile may comprise a profile of protein activity. Up- or down-regulation of gene expression or activity of IFNa-inducible PD markers may be determined by detecting activity of proteins including, but not limited to, detectable phosphorylation activity, de-phosphorylation activity, or cleavage activity.

Furthermore, up-o r down-regulation of gene expression or activity of IFNa-inducible PD markers may be determined by detecting any combination of these gene expression levels or activities.

### 6.7 Patient Samples

Samples may also be obtained from patients in the methods of the disclosure. Samples include any biological fluid or tissue, such as whole blood, saliva, urine, synovial fluid, bone marrow, cerebrospinal fluid, nasal secretions, sputum, amniotic fluid, bronchoalveolar lavage fluid, peripheral blood mononuclear cells, total white blood cells, lymph node cells, spleen cells, tonsil cells, or skin. The samples may be obtained by any means known in the art. VI. Methods of monitoring disease progression

In methods of monitoring disease progression of a patient samples from the patient may be obtained before and after administration of an agent, e.g., an agent that binds to and modulates type I IFN or IFNa activity, or an agent that binds to and does not modulate type I IFN or IFNa activity, or a combination of agents that may or may not include an agent that binds to and modulates type I IFN or IFNa activity. Type I IFN or IFNa inducible PD marker expression profiles are obtained in the (before and after agent administration) samples. The type I IFN or IFNa inducible PD marker expression profiles in the samples are compared. Comparison may be of the number of type I IFN or IFNa inducible PD markers present in the samples or may be of the quantity of type I IFN or IFNa inducible PD markers present in the samples, or any combination thereof. Variance indicating efficacy of the therapeutic agent may be indicated if the number or level (or any combination thereof) of up-regulated type I IFN or IFNa inducible PD markers decreases in the sample obtained after administration of the therapeutic agent relative to the sample obtained before administration of the therapeutic agent. The number of up-regulated type I IFN or IFNa inducible PD markers may decrease by at least 1, at least 2, at least 3, at least 4, at least 5, at least 6, at least 7, at least 8, at least 9, or at least 10 fold. The level of any given up-regulated type I IFN or IFNa inducible PD marker may decrease by at least 10%, at least 20%, at least 25%, at least 30%, at least 35%, at least 40%, at least 50%, at least 60%, at least 70%, at least 80%, at least 90%, or at least 95%. The number of up-regulated type I IFN or IFNa inducible PD markers with decreased levels may be at least 1, at least 2, at least 3, or at least 4. Any combination of decreased number and decreased level of up-regulated type I IFN or IFNa inducible PD markers may indicate efficacy. Variance indicating efficacy of the therapeutic agent may be indicated if the number or level (or any combination thereof) of down-regulated type I IFN or IFNa inducible PD markers decreases in the sample obtained after administration of the therapeutic agent relative to the sample obtained before administration of the therapeutic agent.

The sample obtained from the patient may be obtained prior to a first administration of the agent, i.e., the patient is naive to the agent. Alternatively, the sample obtained from the patient may occur after administration of the agent in the course of treatment. For example, the agent may have been administered prior to the initiation of the monitoring protocol. Following administration of the agent an additional sample may be obtained from the patient and type I IFN or IFNa inducible PD markers in the samples are compared. The samples may be of the same or different type, e.g., each sample obtained may be a blood sample, or each sample obtained may be a serum sample. The type I IFN or IFNa inducible PD markers detected in each sample may be the same, may overlap substantially, or may be similar.

The samples may be obtained at any time before and after the administration of the therapeutic agent. The sample obtained after administration of the therapeutic agent may be obtained at least 2, at least 3, at least 4, at least 5, at least 6, at least 7, at least 8, at least 9, at least 10, at least 12, or at least 14 days after administration of the therapeutic agent. The sample obtained after administration of the therapeutic agent may be obtained at least 2, at least 3, at least 4, at least 5, at least 6, at least 7, or at least 8 weeks after administration of the therapeutic agent. The sample obtained after administration of the therapeutic agent may be obtained at least 2, at least 3, at least 4, at least 5, or at least 6 months following administration of the therapeutic agent.

Additional samples may be obtained from the patient following administration of the therapeutic agent. At least 2, at least 3, at least 4, at least 5, at least 6, at least 7, at least 8, at least 9, at least 10, at least 12, at least 15, at least 20, at least 25 samples may be obtained from the patient to monitor progression or regression of the disease or disorder over time. Disease progression may be monitored over a time period of at least 1 week, at least 2 weeks, at least 3 weeks, at least 4 weeks, at least 5 weeks, at least 6 weeks, at least 7 weeks, at least 2 months, at least 3 months, at least 4 months, at least 5 months, at least 6 months, at least 1 year, at least 2 years, at least 3 years, at least 4 years, at least 5 years, at least 10 years, or over the lifetime of the patient. Additional samples may be obtained from the patient at regular intervals such as at monthly, bi-monthly, once a quarter year, twice a year, or yearly intervals. The samples may be obtained from the patient following administration of the agent at regular intervals. For instance, the samples may be obtained from the patient at one week following each administration of the agent, or at two weeks following each administration of the agent, or at three weeks following each administration of the agent, or at one month following each administration of the agent, or at two months following each administration of the agent. Alternatively, multiple samples may be obtained from the patient following each administration of the agent.

Disease progression in a patient may similarly be monitored in the absence of administration of an agent. Samples may periodically be obtained from the patient having the disease or disorder. Disease progression may be identified if the number of type I IFN or IFNa inducible PD markers increases in a later-obtained sample relative to an earlier obtained sample. The number of type I IFN or IFNa inducible PD markers may increase by at least 1, at least 2, at least 3, at least 4, at least 5, at least 6, at least 7, at least 8, at least 9, or at least 10. Disease progression may be identified if level of any given up-regulated type I IFN or IFNa inducible PD marker increases by at least 10%, at least 20%, at least 25%, at least 30%, at least 35%, at least 40%, at least 50%, at least 60%, at least 70%, at least 80%, at least 90%, or at least 95%. Disease progression may be identified if level of any given down-regulated type I IFN or IFNa inducible PD marker decreases by at least 10%, at least 20%, at least 25%, at least 30%, at least 35%, at least 40%, at least 50%, at least 60%, at least 70%, at least 80%, at least 90%, or at least 95%. The number of up-regulated type I IFN or IFNa inducible PD markers with increased levels may be at least 1, at least 2, at least 3, at least 4, at least 5, at least 6, at least 7, at least 8, at least 9, at least 10, at least 15, at least 20, at least 25, at least 30, or at least 35. The number of down-regulated type I IFN or IFNa inducible PD markers with decreased levels may be at least 1, at least 2, at least 3, at least 4, at least 5, at least 6, at least 7, at least 8, at least 9, at least 10, at least 15, at least 20, at least 25, at least 30, or at least 35. Any combination of increased number and increased level of up-regulated type I IFN or IFNa inducible PD marker may indicate disease progression. Alternatively, or in combination, any combination of decreased number and decreased level of down-regulated type I IFN or IFNa inducible PD marker may indicate disease progression. Disease regression may also be identified in a patient having a disease or disorder, not treated by an agent. In this instance, regression may be identified if the number of type I IFN or IFNa inducible PD markers decreases in a later-obtained sample relative to an earlier obtained sample. The number of type I IFN or IFNa inducible PD markers may decrease by at least 1, at least 2, at least 3, at least 4, at least 5, at least 6, at least 7, at least 8, at least 9, or at least 10. Disease regression may be identified if level of any given up-regulated type I IFN or IFNa inducible PD marker decreases by at least 10%, at least 20%, at least 25%, at least 30%, at least 35%, at least 40%, at least 50%, at least 60%, at least 70%, at least 80%, at least 90%, or at least 95%. Disease regression may be identified if level of any given down-regulated type I IFN or IFNa inducible PD marker increases by at least 10%, at least 20%, at least 25%, at least 30%, at least 35%, at least 40%, at least 50%, at least 60%, at least 70%, at least 80%, at least 90%, or at least 95%. The number of up-regulated type I IFN or IFNa inducible PD markers with decreased levels may be at least 1, at least 2, at least 3, at least 4, at least 5, at least 6, at least 7, at least 8, at least 9, at least 10, at least 15, at least 20, at least 25, at least 30, or at least 35. The number of down-regulated type I IFN or IFNa inducible PD markers with increased levels may be at least 1, at least 2, at least 3, at least 4, at least 5, at least 6, at least 7, at least 8, at least 9, at least 10, at least 15, at least 20, at least 25, at least 30, or at least 35. Disease progression or disease regression may be monitored by obtaining samples over any period of time and at any interval. Disease progression or disease regression may be monitored by obtaining samples over the course of at least 1 week, at least 2 weeks, at least 3 weeks, at least 4 weeks, at least 5 weeks, at least 6 weeks, at least 7 weeks, at least 2 months, at least 3 months, at least 4 months, at least 5 months, at least 6 months, at least 1 year, at least 2 years, at least 3 years, at least 4 years, at least 5 years, at least 10 years, or over the lifetime of the patient. Disease progression or disease regression may be monitored by obtaining samples at least monthly, bi-monthly, once a quarter year, twice a year, or yearly. The samples need not be obtained at strict intervals.

### 6.8 Kits and Probes

The disclosure also encompasses kits and probes. The probes may be any molecule that detects any expression or activity of any gene that may be included in an IFNa-inducible PD marker expression profile.

### 6.9 Subject

The term “subject” is intended to include human and non-human animals, particularly mammals. The subject may be an adult human patient. The subject may be a patient with moderate to severe SLE.

### 6.10 Treatment

The terms “treatment” or “treat” as used herein refer to both therapeutic treatment and prophylactic or preventative measures. Those in need of treatment include subjects having SLE as well as those prone to having SLE or those in which SLE is to be prevented. In some embodiments, the methods disclosed herein can be used to treat SLE.

### 6.11 Administration

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

### 6.12 Doses and Methods of Administration

The method may comprise administering an intravenous dose of anifrolumab or the functional variant thereof to the subject. The intravenous dose may be ≥300 mg anifrolumab or the functional variant thereof. The intravenous dose may be ≤1000 mg. The intravenous dose may be about 300 mg, about 900 mg or about 1000 mg. The intravenous dose may be administered every four weeks (Q4W).

The method may comprise administering a subcutaneous dose of anifrolumab or the functional variant thereof. The subcutaneous dose may be >105 mg and <150 mg anifrolumab or the functional variant thereof. The subcutaneous dose may be ≤135 mg anifrolumab or the functional variant thereof. The subcutaneous dose may be about 120 mg. The subcutaneous dose may be administered in a single administration step. The subcutaneous dose may be administered at intervals of 6-8 days. The subcutaneous dose may be administered once per week. The subcutaneous dose may have a volume of about 0.5 to about 1 m. The subcutaneous dose may have a volume of about 0.8 ml.

### 6.13 Pharmaceutical Composition

The terms “pharmaceutical composition” as used herein refers to a compound or composition capable of inducing a desired therapeutic effect when properly administered to a subject. In some embodiments, the disclosure provides a pharmaceutical composition comprising a pharmaceutically acceptable carrier and a therapeutically effective amount of at least one antibody of the disclosure.

The terms “pharmaceutically acceptable carrier” or “physiologically acceptable carrier” as used herein refer to one or more formulation materials suitable for accomplishing or enhancing the delivery of one or more antibodies of the disclosure.

### 6.14 Antigen-Binding Fragment

The term “antigen-binding fragment” refers to one or more fragments of an antibody that retain(s) the ability to specifically bind to the antigen. Examples of antigen-binding fragments include the following: Fab fragment, F (ab′)2 fragment, Fd fragment, Fv fragment, dAb fragment, as well as a scFv.

### 6.15 Systemic Lupus Erythematosus (SLE)

Systemic lupus erythematosus (SLE) is a chronic, multisystemic, disabling autoimmune rheumatic disease of unknown etiology. Systemic lupus erythematosus predominantly affects women of childbearing years with a recent review reporting the female-to-male ratio in the childbearing years to be about 12:1. Accurate data on the current incidence and prevalence of SLE are largely lacking, however there are numerous indications that SLE is more common in non-Caucasian populations; for example, in the United States of America (USA), SLE is more prevalent in African-Americans, Hispanics, and Asians than Caucasians. As a result, SLE prevalence varies from country to country. In addition, variability of SLE prevalence within countries appears to be dependent upon racial, genetic differences, complex socioeconomic factors and age; the incidence of disease in females is usually highest between 15-44 years of age.

Clinical manifestations of SLE can include constitutional symptoms, alopecia and rashes, serositis, inflammatory arthritis, renal disease, systemic vasculitis, lymphadenopathy, splenomegaly, hemolytic anemia, cognitive dysfunction and other central nervous system (CNS) involvement. These disease manifestations cause a significant burden of illness and can cause reduced physical function, loss of employment, lower health-related quality of life (HRQoL), and a lifespan shortened by about 10 years. Increased hospitalizations and side effects of medications including chronic oral corticosteroids (OCS) and other immunosuppressive treatments add to disease burden in SLE At this time, belimumab is the only new treatment for SLE that has been approved by the Food and Drug Administration (FDA) in about 50 years since hydroxychloroquine was approved for use in discoid lupus and SLE. The existing standard of care treatment for SLE (SOC SLE) otherwise consists of off-label medications. lupus erythematosus.

### 6.16 CLASI (Cutaneous Lupus Erythematosus Disease Area and Severity Index)

CLASI is a tool used to measure disease severity and response to treatment. A 4-point or 20% decrease in CLASI activity score is commonly viewed as a cut-off for classifying subjects as responders to treatment. In particular embodiments, treatment using anifrolumab results in at least 50% reduction of a subject's CLASI score compared to the subject's baseline score.

The CLASI is a validated index used for assessing the cutaneous lesions of SLE and consists of 2 separate scores: the first summarizes the inflammatory activity of the disease; the second is a measure of the damage done by the disease. The activity score takes into account erythema, scale/hypertrophy, mucous membrane lesions, recent hair loss, and nonscarring alopecia. The damage score represents dyspigmentation, scarring/atrophy/panniculitis, and scarring of the scalp. Subjects are asked if their dyspigmentation lasted 12 months or longer, in which case the dyspigmentation score is doubled. Each of the above parameters is measured in 13 different anatomical locations, included specifically because they are most often involved in cutaneous lupus erythematosus (CLE). The most severe lesion in each area is measured.

In particular embodiments, treatment using anifrolumab reduces a subject's CLASI score by at least week 8, week 12, week 24, week 36, week 48, or week 52 of treatment. In particular embodiments, treatment using anifrolumab reduces a subject's CLASI score by at least week 8. In particular embodiments, treatment using anifrolumab reduces a subject's CLASI score by at least week 12.

In particular embodiments provided herein is a method of treating systemic lupus erythematosus in a subject in need thereof, comprising administering to the subject a therapeutically effective amount of anifrolumab, wherein the treatment results in a reduction in the Cutaneous Lupus Erythematosus Disease Area and Severity Index (CLASI) score compared to a patient receiving placebo.

### 6.17 Oral Corticosteroids

Oral corticosteroids include prednisone, cortisone, hydrocortisone, methylprednisolone, prednisolone and triamcinolone.

In particular embodiments provided herein is a method of treating systemic lupus erythematosus in a subject in need thereof, wherein the subject is being treated with oral corticosteroids, comprising administering to the subject a therapeutically effective amount of anifrolumab, wherein the treatment results in a reduction in oral corticosteroid dosage in the subject to at least ≤5.5 mg/day, ≤6.5 mg/day, ≤7.5 mg/day, or ≤8.5 mg/day. In particular embodiments, the reduction in oral corticosteroid dosage in the subject is reduced to at least ≤7.5 mg/day. In particular embodiments, the treatment results in a reduction in oral corticosteroid dosage in the subject from ≥10 mg/day to ≤7.5 mg/day.

In particular embodiments provided herein is a method of treating systemic lupus erythematosus in a subject in need thereof, wherein the subject is being treated with oral corticosteroids, comprising administering to the subject a therapeutically effective amount of anifrolumab, wherein the treatment results in a reduction in oral corticosteroid dosage in the subject to at least ≤5.5 mg/day, ≤6.5 mg/day of prednisone or prednisone equivalent dose, ≤7.5 mg/day of prednisone or prednisone equivalent dose, or ≤8.5 mg/day of prednisone or prednisone equivalent dose. In particular embodiments, the reduction in oral corticosteroid dosage in the subject is reduced to at least ≤7.5 mg/day. In particular embodiments, the treatment results in a reduction in oral corticosteroid dosage in the subject from ≥10 mg/day to ≤7.5 mg/day of prednisone or prednisone equivalent dose.

Examples of equivalent doses of oral prednisone are shown in Table 6-3.

TABLE 6-3
Examples of equivalent doses of oral prednisone

| | Oral Prednisone | | | | | | | | | |
|---|---|---|---|---|---|---|---|---|---|---|
| | and Equivalents | | | | | | | | | |
| Oral | Equivalent Dose | | | | | | | | | |
| Prednisone | 7.5 mg | | 10 mg | | 20 mg | | 30 mg | | 40 mg | |
| Cortisone | 37.5 | mg | 50 | mg | 100 | mg | 150 | mg | 200 | mg |
| Hydrocortisone | 30 | mg | 40 | mg | 80 | mg | 120 | mg | 160 | mg |
| Methylpred- | 6 | mg | 8 | mg | 16 | mg | 24 | mg | 32 | mg |
| nisolone | | | | | | | | | | |
| Prednisolone | 7.5 | mg | 10 | mg | 20 | mg | 30 | mg | 40 | mg |
| Triamcinolone | 6 | mg | 8 | mg | 16 | mg | 24 | mg | 32 | mg |

### 6.18 Type I IFN Inhibitors

### 6.18.1 Anifrolumab

Type I interferons (IFN) are cytokines that form a crucial link between innate and adaptive immunity and are implicated in SLE by genetic susceptibility data and upregulated interferon-stimulated gene expression in the majority of SLE patients⁹.

Anifrolumab (MEDI-546, “ANI”, “anifro”) inhibits binding of type I IFN to type I interferon receptor (IFNAR) and inhibits the biologic activity of all type I IFNs. Anifrolumab (MEDI-546) is a human immunoglobulin G1 kappa (lgG1_{K}) monoclonal antibody (mAb) directed against subunit 1 of the type I interferon receptor (IFNAR1). It is composed of 2 identical light chains and 2 identical heavy chains, with an overall molecular weight of approximately 148 kDa. Disclosure related to anifrolumab can be found in U.S. Pat. Nos. 7,662,381 and 9,988,459, which are incorporated herein by reference.

Anifrolumab is an IFNAR-blocking (antagonistic) antibody, and blocks the activity of the receptor's ligands, namely type I interferons such as interferon-α and interferon-β. Anifrolumab thus provides for downregulation of IFNAR signaling, and thus suppression of IFN-inducible genes.

TABLE 6-4
Anifrolumab sequences

| | |
|---|---|
| Anifrolumab VH (SEQ | EVQLVQSGAEVKKPGESLKISCKGSGYIFTNYWIAWVRQMPGKGLESMGI |
| ID NO: 1) | IYPGDSDIRYSPSFQGQVTISADKSITTAYLQWSSLKASDTAMYYCARHD |
| | IEGFDYWGRGTLVTVSS |
| Anifrolumab VL (SEQ | EIVLTQSPGTLSLSPGERATLSCRASQSVSSSFFAWYQQKPGQAPRLLIY |
| ID NO: 2) | GASSRATGIPDRLSGSGSGTDFTLTITRLEPEDFAVYYCQQYDSSAITFG |
| | QGTRLEIK |
| HCDR1 (SEQ ID NO: 3) | NYWIA |
| HCDR2 (SEQ ID NO: 4) | IIYPGDSDIRYSPSFQG |
| HCDR3 (SEQ ID NO: 5) | HDIEGFDY |
| LCDR1 (SEQ ID NO: 6) | RASQSVSSSFFA |
| LCDR2 (SEQ ID NO: 7) | GASSRAT |
| LCDR3 (SEQ ID NO: 8) | QQYDSSAIT |
| Light chain constant | RTVAAPSVFIFPPSDEQLKSGTASVVCLLNNFYPREAKVQWKVDNALQSGNSQE |
| region (SEQ ID NO: 9) | SVTEQDSKDSTYSLSSTLTLSKADYEKHKVYACEVTHQGLSSPVTKSFNRGEC |
| Heavy chain constant | ASTKGPSVFPLAPSSKSTSGGTAALGCLVKDYFPEPVTVSWNSGALTSGV |
| region | HTFPAVLQSSGLYSLSSVVTVPSSSLGTQTYICNVNHKPSNTKVDKRVEP |
| (SEQ ID NO: 10) | KSCDKTHTCPPCPAPEFEGGPSVFLFPPKPKDTLMISRTPEVTCVVVDVS |
| | HEDPEVKFNWYVDGVEVHNAKTKPREEQYNSTYRVVSVLTVLHQDWLNGK |
| | EYKCKVSNKALPASIEKTISKAKGQPREPQVYTLPPSREEMTKNQVSLTC |
| | LVKGFYPSDIAVEWESNGQPENNYKTTPPVLDSDGSFFLYSKLTVDKSRW |
| | QQGNVFSCSVMHEALHNHYTQKSLSLSPGK |
| Heavy chain | EVQLVQSGAEVKKPGESLKISCKGSGYIFTNYWIAWVRQMPGKGLESMGI |
| (SEQ ID NO: 11) | IYPGDSDIRYSPSFQGQVTISADKSITTAYLQWSSLKASD |
| | TAMYYCARHD |
| | IEGFDYWGRGTLVTVSSASTKGPSVFPLAPSSKSTSGGTAALGCLVKDYF |
| | PEPVTVSWNSGALTSGVHTFPAVLQSSGLYSLSSVVTVPS |
| | SSLGTQTYIC |
| | NVNHKPSNTKVDKRVEPKSCDKTHTCPPCPAPEFEGGPSVFLFPPKPKDT |
| | LMISRTPEVTCVVVDVSHEDPEVKFNWYVDGVEVHNAKTK |
| | PREEQYNSTYRVVSVLTVLHQDWLNGKEYK CKVSNKALPA |
| | SIEKTISKAK |
| | GQPREPQVYTLPPSREEMTKNQVSLTCLVKGFYPSDIAVEWESNGQPENN |
| | YKTTPPVLDSDGSFFLYSKLTVDKSRWQQGNVFSCSVMHEALHNHYTQKS |
| | LSLSPGK |
| Light chain | EIVLTQSPGTLSLSPGERATLSCRASQSVS SSFFAWYQQK |
| (SEQ ID NO: 12) | PGQAPRLLIY GASSRATGIPDRLSGSGSGT DFTLTITRLE |
| | PEDFAVYYCQ QYDSSAITFG |
| | QGTRLEIKRTVAAPSVFIFPPSDEQLKSGT ASVVCLLNNF |
| | YPREAKVQWK |
| | VDNALQSGNSQESVTEQDSKDSTYSLSSTLTLSKADYEKHKVYACEVTHQ |
| | GLSSPVTKSFNRGEC |

Thus, anifrolumab is an antibody comprising an HCDR1, HCDR2 and HCDR3 of SEQ ID NO: 3, SEQ ID NO: 4, and SEQ ID NO: 5, respectively (or functional variant thereof); and an LCDR1, LCDR2 and LCDR3 of SEQ ID NO: 6, SEQ ID NO: 7, and SEQ ID NO: 8, respectively (or functional variant thereof). In more detail, anifrolumab as referred to herein is an antibody comprising a VH of SEQ ID NO: 1 and a VL of SEQ ID NO: 2 (or functional variant thereof).

The present invention encompasses the antibodies defined herein having the recited CDR sequences or variable heavy and variable light chain sequences (reference (anifrolumab) antibodies), as well as functional variants thereof. A “functional variant” binds to the same target antigen as the reference (anifrolumab) antibody. The functional variants may have a different affinity for the target antigen when compared to the reference antibody, but substantially the same affinity is preferred.

In one embodiment functional variants of a reference (anifrolumab) antibody show sequence variation at one or more CDRs when compared to corresponding reference CDR sequences. Thus, a functional antibody variant may comprise a functional variant of a CDR. Where the term “functional variant” is used in the context of a CDR sequence, this means that the CDR has at most 2, preferably at most 1 amino acid differences when compared to a corresponding reference CDR sequence, and when combined with the remaining 5 CDRs (or variants thereof) enables the variant antibody to bind to the same target antigen as the reference (anifrolumab) antibody, and preferably to exhibit the same affinity for the target antigen as the reference (anifrolumab) antibody.

Without wishing to be bound by theory, since anifrolumab targets (e.g. blocks or antagonizes) IFNAR, it is believed that anifrolumab treats a disease (such as SLE) by blocking signaling initiated by type I interferons (IFNs). Type I IFNs are known to be important drivers of inflammation (e.g. by coordinating the type I interferon response), and thus play a pivotal role in the immune system. However, dysregulation of type I IFN-signaling can lead to aberrant (e.g. aberrantly high) levels of inflammation, and autoimmunity. Such dysregulation of type I IFN interferons has been reported in numerous autoimmune diseases.

For example, a variant of the reference (anifrolumab) antibody may comprise:
a heavy chain CDR1 having at most 2 amino acid differences when compared to SEQ ID NO: 3;
a heavy chain CDR2 having at most 2 amino acid differences when compared to SEQ ID NO: 4;
a heavy chain CDR3 having at most 2 amino acid differences when compared to SEQ ID NO: 5;
a light chain CDR1 having at most 2 amino acid differences when compared to SEQ ID NO: 6;
a light chain CDR2 having at most 2 amino acid differences when compared to SEQ ID NO: 7; and
a light chain CDR3 having at most 2 amino acid differences when compared to SEQ ID NO: 8;
wherein the variant antibody binds to the target of anifrolumab (e.g. IFNAR) and preferably with the same affinity.

Preferably, a variant of the reference (anifrolumab) antibody may comprise:
a heavy chain CDR1 having at most 1 amino acid difference when compared to SEQ ID NO: 3;
a heavy chain CDR2 having at most 1 amino acid difference when compared to SEQ ID NO: 4;
a heavy chain CDR3 having at most 1 amino acid difference when compared to SEQ ID NO: 5;
a light chain CDR1 having at most 1 amino acid differences when compared to SEQ ID NO: 6;
a light chain CDR2 having at most 1 amino acid difference when compared to SEQ ID NO: 7; and
a light chain CDR3 having at most 1 amino acid difference when compared to SEQ ID NO: 8;
wherein the variant antibody binds to the target of anifrolumab (e.g. IFNAR, also referred to as IFNAR1 and IFNR) and preferably with the same affinity.

In one embodiment a variant antibody may have at most 5, 4 or 3 amino acid differences total in the CDRs thereof when compared to a corresponding reference (anifrolumab) antibody, with the proviso that there is at most 2 (preferably at most 1) amino acid differences per CDR. Preferably a variant antibody has at most 2 (more preferably at most 1) amino acid differences total in the CDRs thereof when compared to a corresponding reference (anifrolumab) antibody, with the proviso that there is at most 2 amino acid differences per CDR. More preferably a variant antibody has at most 2 (more preferably at most 1) amino acid differences total in the CDRs thereof when compared to a corresponding reference (anifrolumab) antibody, with the proviso that there is at most 1 amino acid difference per CDR.

The amino acid difference may be an amino acid substitution, insertion or deletion. In one embodiment the amino acid difference is a conservative amino acid substitution as described herein.

In one embodiment a variant antibody may have at most 5, 4 or 3 amino acid differences total in the framework regions thereof when compared to a corresponding reference (anifrolumab) antibody, with the proviso that there is at most 2 (preferably at most 1) amino acid differences per framework region. Preferably a variant antibody has at most 2 (more preferably at most 1) amino acid differences total in the framework regions thereof when compared to a corresponding reference (anifrolumab) antibody, with the proviso that there is at most 2 amino acid differences per framework region. More preferably a variant antibody has at most 2 (more preferably at most 1) amino acid differences total in the framework regions thereof when compared to a corresponding reference (anifrolumab) antibody, with the proviso that there is at most 1 amino acid difference per framework region.

Thus, a variant antibody may comprise a variable heavy chain and a variable light chain as described herein, wherein:
the heavy chain has at most 14 amino acid differences (at most 2 amino acid differences in each CDR and at most 2 amino acid differences in each framework region) when compared to a heavy chain sequence herein; and
the light chain has at most 14 amino acid differences (at most 2 amino acid differences in each CDR and at most 2 amino acid differences in each framework region) when compared to a light chain sequence herein;
wherein the variant antibody binds to the same target antigen as the reference (anifrolumab) antibody (e.g. IFNAR) and preferably with the same affinity.

The variant heavy or light chains may be referred to as “functional equivalents” of the reference heavy or light chains.

In one embodiment a variant antibody may comprise a variable heavy chain and a variable light chain as described herein, wherein:
the heavy chain has at most 7 amino acid differences (at most 1 amino acid difference in each CDR and at most 1 amino acid difference in each framework region) when compared to a heavy chain sequence herein; and
the light chain has at most 7 amino acid differences (at most 1 amino acid difference in each CDR and at most 1 amino acid difference in each framework region) when compared to a light chain sequence herein;
wherein the variant antibody binds to the same target antigen as the reference (anifrolumab) antibody (e.g. IFNAR) and preferably with the same affinity.

Functional variants of anifrolumab are sequence variants that perform the same function as anifrolumab. Functional variants of anifrolumab are variants that bind the same target as anifrolumab and have the same effector function as anifrolumab. Functional anifrolumab variants include antigen-binding fragments of anifrolumab and antibody and immunoglobulin derivatives of anifrolumab. Functional variants include biosimilars and interchangeable products. The terms biosimilar and interchangeable product are defined by the FDA and EMA. The term biosimilar refers to a biological product that is highly similar to an approved (e.g. FDA approved) biological product (reference product, e.g. anifrolumab) in terms of structure and has no clinically meaningful differences in terms of pharmacokinetics, safety and efficacy from the reference product. The presence of clinically meaningful differences of a biosimilar may be assessed in human pharmacokinetic (exposure) and pharmacodynamic (response) studies and an assessment of clinical immunogenicity. An interchangeable product is a biosimilar that is expected to produce the same clinical result as the reference product in any given patient.

Thus, in one embodiment the type I interferon receptor inhibitor is anifrolumab or a functional variant thereof.

Functional variants of anifrolumab include the antibodies described in WO 2018/023976 A1, incorporated herein by reference (Table 6-5).

TABLE 6-5
anti-IFNAR antibody sequences

| | | | |
|---|---|---|---|
| H15D10 | VH | EVQLVQSGAE VKKPGESLRISC | SEQ ID |
| | | KGSGYTFT NYWVAWVRQM | NO: 13 |
| | | PGKGLESMGI IYPGDSDTRY | |
| | | SPSFQGHVTI SADKSISTAY | |
| L8C3 | VL | DIQMTQSPSSLSASLGDRVTITCRASQNVG | SEQ ID |
| | | NYLNWYQQKPGKAPKLLIYRASNLASGVPS | NO: 14 |
| | | RFSGSGSGTDFTLTISSLQPEDFATYYCQQ | |
| | | MEHAPPTFGQGTKVEIKR | |
| L16C11 | VL | EIVLTQSPGTLSLSPGERATLSCRASQSVI | SEQ ID |
| | | GYYLAWYQQKPGQAPRLLIYSVSTLASGIP | NO: 15 |
| | | DRFSGSGSGTDFTLTISRLEPEDFAVYYCQ | |
| | | QYYRFPITFGQGTKVEIK | |
| H19B7 | VH | EVQLVQSGAE VKKPGESLRI | SEQ ID |
| | | SCKGSGYTFT NYWMAWVRQM | NO: 16 |
| | | PGKGLESMGI IYPSDSDTRY | |
| | | SPSFQGHVTI SADKSISTAY | |
| | | LOWSSLKASD TAMYYCARHD | |
| | | VEGYDYWGQG TLVTVSS | |

Functional variants include antibodies comprising the VH amino acid sequence SEQ ID NO: 13. Functional variants include antibodies comprising the VH amino acid sequence SEQ ID NO: 16. Functional variants include antibodies comprising the VL amino acid sequence SEQ ID NO: 14. Functional variants include antibodies comprising the VL amino acid sequence SEQ ID NO: 15. Functional variants include antibodies comprising the VL amino acid sequence SEQ ID NO: 16. Functional variants include antibodies comprising the VH sequence SEQ ID NO: 13 and VL amino acid sequence SEQ ID NO: 16. Functional variants include antibodies comprising the VH sequence SEQ ID NO: 13 and VL amino acid sequence SEQ ID NO: 15. Functional variants include antibodies comprising the VH sequence SEQ ID NO: 16 and VL amino acid sequence SEQ ID NO: 15. Functional variants include antibodies comprising the VH sequence SEQ ID NO: 16 and VL amino acid sequence SEQ ID NO: 14.

IFNAR inhibitors may be a monoclonal antibody comprising the VH amino acid sequence SEQ ID NO: 13. The anti-IFNAR antibodies may comprise the VH amino acid sequence SEQ ID NO: 16. The anti-IFNAR antibodies may comprise the VL amino acid sequence SEQ ID NO: 14. The anti-IFNAR antibodies may comprise the VL amino acid sequence SEQ ID NO: 15. The anti-IFNAR antibodies may comprise the VL amino acid sequence SEQ ID NO: 16. The anti-IFNAR antibodies may comprise the VH sequence SEQ ID NO: 13 and VL amino acid sequence SEQ ID NO: 16. The anti-IFNAR antibodies may comprise the VH sequence SEQ ID NO: 13 and VL amino acid sequence SEQ ID NO: 15. The anti-IFNAR antibodies may comprise the VH sequence SEQ ID NO: 16 and VL amino acid sequence SEQ ID NO: 15. The anti-IFNAR antibodies may comprise the VH sequence SEQ ID NO: 16 and VL amino acid sequence SEQ ID NO: 14.

### 6.18.2 Sifalimumab

Sifalimumab (MEDI-545) is a fully human, immunoglobulin G1_{K} monoclonal antibody that binds to and neutralizes the majority of IFN-α subtypes¹⁰. Sifalimumab is described U.S. Pat. No. 7,741,449, which is incorporated herein by reference in its entirety. The efficacy and safety of sifalimumab were assessed in a phase IIb, randomised, double-blind, placebo-controlled study (NCT01283139) of adults with moderate to severe active systemic lupus erythematosus (SLE). 431 patients were randomised and received monthly intravenous sifalimumab (200 mg, 600 mg or 1200 mg) or placebo in addition to standard-of-care medications. The primary efficacy end point was the percentage of patients achieving an SLE responder index response at week 52. Compared with placebo, a greater percentage of patients who received sifalimumab (all dosages) met the primary end point (placebo: 45.4%; 200 mg: 58.3%; 600 mg: 56.5%; 1200 mg 59.8%).

Administration of sifalimumab to SLE patients having an elevated type I IFN signature neutralises the IFNGS^{11,12}.

### 6.19 Tender and Swollen Joints

The swollen and tender joint count is based on left and right shoulder, elbow, wrist, metacarpophalangeal (MCP) 1, MCP2, MCP3, MCP4, MCP5, proximal interphalangeal (PIP) 1, PIP2, PIP3, PIP4, PIP5 joints of the upper extremities and left and right knee of the lower extremities. An active joint for the joint count assessment is defined as a joint with tenderness and swelling.

In particular embodiments provided herein is method of treating systemic lupus erythematosus in a subject in need thereof, comprising administering to the subject a therapeutically effective amount of anifrolumab, wherein the treatment results in at least 50% improvement from baseline value of tender joint count and swollen joint count compared to a patient receiving placebo.

### 6.20 Dosage Regimes

The dose of the anifrolumab to be administered to the subject may vary depending, in part, upon the size (body weight, body surface, or organ size) and condition (the age and general health) of the subject.

In particular embodiments, the subject is administered one or more fixed doses of anifrolumab, wherein the dose is 150 mg, 200 mg, 250 mg, 300 mg, or 350 mg. In some embodiments, the subject is administered one or more fixed doses of anifrolumab wherein the dose is 300 mg.

In particular embodiments, anifrolumab is administered over a two-week treatment period, over a four-week treatment period, over a six-week treatment period, over an eight-week treatment period, over a twelve-week treatment period, over a twenty-four-week treatment period, or over a one-year or more treatment period. In particular embodiments, anifrolumab is administered over a three-week treatment period, over a six-week treatment period, over a nine-week treatment period, over a twelve-week treatment period, over a twenty-four-week treatment period, or over a one-year or more treatment period. In particular embodiments, anifrolumab is administered for at least 52 weeks.

In particular embodiments, anifrolumab is administered every week, every two weeks, every four weeks, every six weeks, every eight weeks, every ten weeks, or every twelve weeks.

### 6.21 Administration Methods

When used for in vivo administration, the formulations of the disclosure should be sterile. The formulations of the disclosure may be sterilized by various sterilization methods, including, for example, sterile filtration or radiation. In one embodiment, the formulation is filter sterilized with a presterilized 0.22-micron filter. Sterile compositions for injection can be formulated according to conventional pharmaceutical practice as described in “Remington: The Science & Practice of Pharmacy,” 21st ed., Lippincott Williams & Wilkins, (2005).

In some embodiments, type I IFN inhibitor can be formulated for particular routes of administration, such as oral, nasal, pulmonary, topical (including buccal and sublingual), rectal, vaginal, and/or parenteral administration. The terms “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

In some embodiments, anifrolumab can be formulated for particular routes of administration, such as oral, nasal, pulmonary, topical (including buccal and sublingual), rectal, vaginal, and/or parenteral administration. The terms “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

### 6.22 Formulations

The formulations can be presented in unit dosage form and can be prepared by any method known in the art of pharmacy. Actual dosage levels of the active ingredients in the formulation of the present disclosure may be varied so as to obtain an amount of the active ingredient which is effective to achieve the desired therapeutic response for a particular subject, composition, and mode of administration, without being toxic to the subject (e.g., “a therapeutically effective amount”). Dosages can also be administered via continuous infusion (such as through a pump). The administered dose may also depend on the route of administration.

The pharmaceutical composition may comprise about 150 mg/mL anifrolumab. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may have a pH of 5.9.

### 6.23 Interferon Test

Type I IFN is considered important in SLE disease pathogenesis and inhibition of this pathway is targeted by anifrolumab. To understand the relationship between type I IFN expression and response to anti-IFN therapy, it is necessary to know if a subject's disease is driven by type I IFN activation. However, direct measurement of the target protein remains a challenge. As such, a transcript-based marker was developed to evaluate the effect of over expression of the target protein on a specific set of mRNA markers. The expression of these markers is easily detected in whole blood and demonstrates a correlation with expression in diseased tissue such as skin in SLE. The bimodal distribution of the transcript scores for SLE subjects supports defining an IFN test high and low subpopulation (FIG. 1A). The type I IFN test is described in WO2011028933 A1, which is incorporated herein by reference in its entirety.

### 6.24 SRI (Systemic Lupus Erythematosus Responder Index of ≥4)

A subject achieves SRI(4) if all of the following criteria are met:
Reduction from baseline of ≥4 points in the SLEDAI-2K;
No new organ system affected as defined by 1 or more BILAG-2004 A or 2 or more
BILAG-2004 B items compared to baseline using BILAG-2004;
No worsening from baseline in the subjects' lupus disease activity defined by an increase ≥0.30 points on a 3-point PGA VAS.

SRI (X) (X=5, 6, 7, or 8) is defined by the proportion of subjects who meet the following criteria:
Reduction from baseline of ≥X points in the SLEDAI-2K;
No new organ systems affected as defined by 1 or more BILAG-2004 A or 2 or
more BILAG-2004 B items compared to baseline using BILAG-2004;
No worsening from baseline in the subjects' lupus disease activity defined by an
increase ≥0.30 points on a 3-point PGA VAS

### 6.25 BILAG-2004, British Isles Lupus Assessment Group-2004

The BILAG-2004 is a translational index with 9 organ systems (General, Mucocutaneous, Neuropsychiatric, Musculoskeletal, Cardiorespiratory, Gastrointestinal, Ophthalmic, Renal and Haematology) that is able to capture changing severity of clinical manifestations. It has ordinal scales by design and does not have a global score; rather it records disease activity across the different organ systems at a glance by comparing the immediate past 4 weeks to the 4 weeks preceding them. It is based on the principle of physicians' intention to treat and categorises disease activity into 5 different levels from A to E:
Grade A represents very active disease requiring immunosuppressive drugs and/or a prednisone dose of >20 mg/day or equivalent
Grade B represents moderate disease activity requiring a lower dose of corticosteroids, topical steroids, topical immunosuppressives, antimalarials, or NSAIDs
Grade C indicates mild stable disease
Grade D implies no disease activity but the system has previously been affected
Grade E indicates no current or previous disease activity

Although the BILAG-2004 was developed based on the principle of intention to treat, the treatment has no bearing on the scoring index. Only the presence of active manifestations influences the scoring.

### 6.26 BICLA: BILAG-Based Composite Lupus Assessment (BICLA)

BICLA is a composite index that was originally derived by expert consensus of disease activity indices. BICLA response is defined as (1) at least one gradation of improvement in baseline BILAG scores in all body systems with moderate or severe disease activity at entry (e.g., all A (severe disease) scores falling to B (moderate), C (mild), or D (no activity) and all B scores falling to C or D); (2) no new BILAG A or more than one new BILAG B scores; (3) no worsening of total SLEDAI score from baseline; (4) no significant deterioration (≤10%) in physicians global assessment; and (5) no treatment failure (initiation of non-protocol treatment).

Particularly, a subject is a BICLA responder if the following criteria are met:
Reduction of all baseline BILAG-2004 A to B/C/D and baseline BILAG-2004 B to C/D, and no BILAG-2004 worsening in other organ systems, as defined by 1 new BILAG-2004 A or more than 1 new BILAG-2004 B item;
No worsening from baseline in SLEDAI-2K as defined as an increase from baseline of >0 points in SLEDAI-2K;
No worsening from baseline in the subjects' lupus disease activity defined by an increase ≥0.30 points on a 3-point PGA VAS;
No discontinuation of investigational product or use of restricted medications beyond the protocol-allowed threshold before assessment

In particular embodiments, treatment using anifrolumab improves a subject's BICLA response rate by at least week 8, week 12, week 24, week 36, week 48, or week 52 of treatment. In particular embodiments, treatment using anifrolumab improves a subject's BICLA response rate by at least week 8.

In particular embodiments, while the subject shows improvement in the BICLA response, the subject does not show improvement in the Systemic Lupus Erythematosus Responder Index (SRI)4 score.

In particular embodiments provided herein is a method of treating systemic lupus erythematosus in a subject in need thereof, comprising administering to the subject a therapeutically effective amount of anifrolumab, wherein the treatment results in an improvement of the BILAG-Based Composite Lupus Assessment (BICLA) response rate compared to a patient receiving placebo. The improvement of the BILAG response rate may be statistically significant. The improvement of the BILAG response rate may be statistically significant after multiplicity adjustment. The improvement of the BILAG response rate may be statistically significant, wherein statistical significance is determined by p<0.05 or p<0.005.

### 6.27 Anti-BAFF Antibodies

### 6.27.1 Belimumab

Belimumab is an anti-BAFF antibody approved for the treatment of SLE patients with active, autoantibody-positive disease, who are already on standard therapy. Belimumab selectively binds to soluble human B lymphocyte stimulator protein (BAFF, also known as BLysS). Belimumab is a fully human lgG1λ recombinant monoclonal antibody directed against BLyS. Specific binding of belimumab with the soluble BLyS prevents the interaction of BLys with its receptors and decreases B-cell survival and production of autoantibodies.

Belimumab sequences are shown in Table 6-6.

TABLE 6-6
Anti-BAFF sequences

| | |
|---|---|
| Belimumab VH | QVQLQQSGAE VKKPGSSVRV SCKASGG FN |
| | NNAINWVRQA PGQGLE GG I IP FGTAKY |
| | SQNFQGRV I TADESTGTAS MELSSLRSED |
| | TAVYYCARSR DLLLFPHHAL SPWGRGTMVT VSS |
| Belimumab VL | SSELTQDPAV SVALGQTVRV TCQGDSLRSY |
| | YASWYQQKPG QAPVLVIYGK NNRP FSGSSSGNTA |
| | SLTITGAQAE DEADYYCSSR DSSGNHWVFG |
| | GGTELTVLG |
| Tabalumab VH | MKHLWFFLLL VAAPRWVLSQ VQLQQWGAGL |
| | LKPSETLSLT CAVYGGSFSG YYWSWIRQPP |
| | GKGLEWIGEI NHSGSTNYNP SLKSRVTISV |
| | DTSKNQFSLK LSSVTAADTA VYYCARGYYD |
| | ILTGYYYYFD YWGQGTLVTV SS |
| Tabalumab VL | EIVLTQSPAT LSLSPGERAT LSCRASQSVS |
| | RYLAWYQQKP GQAPRLLIYD ASNRATGIPA |
| | RFSGSGSGTD STLTISSLEP EDFAVYYCQQ |
| | RSNWPRTFGO GTKVEIKRT |
| Belimumab heavy chain | QVQLQQSGAE VKKPGSSVRV SCKASGGTFN |
| | NNAINWVRQA PGQGLEWMGG I IPMFGTAKY |
| | SQNFQGRVAI TADESTGTAS MELSSLRSED |
| | TAVYYCARSR DLLLFPHHAL SPWGRGTMVT |
| | VSSASTKGPS VFPLAPSSKS TSGGTAALGC |
| | LVKDYFPEPV TVS NSGALT SGVHTFPAVL |
| | QSSGLYSLSS VVTVPSSSLG TQTYICNVNH |
| | KPSNTKVDKK VEPKSCDKTH TCPPCPAPEL |
| | LGGPSVFLFP PKPKDTLMI S RTPEVTCVVV |
| | DVSHEDPEVK FNWYVDGVEV HNAKTKPREE |
| | QYNSTYRVVS VLTVLHQDWL NGKEYKCKVS |
| | NKALPAPIEK TI SKAKGQPR EPQVYTLPPS |
| | RDELTKNQVS LTCLVKGFYP SDIAVEWESN |
| | GQPENNYKTT PPVLDSDGSF FLYSKLTVDK |
| | SRWQQGNVFS CSVMHEALHN HYTQKSLSLS PGK |
| Belimumab light chain | SSELTQDPAV SVALGQTVRV TCQGDSLRSY |
| | YASWYQQKPG QAPVLVIYGK NNRPSGI PDR |
| | FSGSSSGNTA SLTITGAQAE DEADYYCSSR |
| | DSSGNHWVFG GGTELTVLGQ PKAAPSVTLF |
| | PPSSEELQAN KATLVCLISD FYPGAVTVAW |
| | KADSSPV AG VETTTPSKQS NNKYAASSYL |
| | SLTPEQWKSH RSYSCQVTHE GSTVEKTVAP TECS |
| Tabalumab heavy chain | QVQLQQWGAG LLKPSETLSL TCA VYGGSFS |
| | GYYWSWIRQP PGKGLEWIGE I HSGSTNYN |
| | PSLKSRVTIS VDTSKNQFSL KLSSVTAADT |
| | AVYYCARGYY DILTGYYYYF DYWGQGTLVT |
| | VSSASTKGPS VFPLAPCSRS TSESTAALGC |
| | LVKDYFPEPV TVSWNSGALT SGVHTFPAVL |
| | QSSGLYSLSS VVTVPSSSLG TKTYTCNVDH |
| | KPSQTKVDKR VESKYGPPCP PCPAPEFLGG |
| | PSVFLFPPKP KDTLMISRTP EVTCVVVDVS |
| | QEDPEVQFNW YVDGVEVHNA KTKPREEQFN |
| | STYRVVSVLT VLHQDWLNGK EYKCKVSNKG |
| | LPSSIEKTIS K.AKGQPREPQ VYTLPPSQEE |
| | MTKNQVSLTC LVKGFYPSDI AVEWESNGQP |
| | ENNYKTTPPV LDSDGSFFLY |
| | QEGNVFSCSV MHEALHNHYT QKSLSLSLGK |
| Tabalumab light chain | EIVLTQSPAT LSLSPGERAT LSCRASQSVS |
| | RYLAWYQQKP GQAPRLLIYD ASNRATGIPA |
| | RFSGSGSGTD STLTISSLEP EDFAVYYCQQ |
| | RSNWPRTFGQ GTKVEIKRTV AAPSVFIFPP |
| | SDEQLKSGTA SVVCLLNNFY PREAKVQWKV |
| | DNALQSGNSQ ESVTEQDSKD STYSLSNTLT |
| | LSKADYEKHK VYACEVTHQG LSSPVTKSEN RGEC |
| Belimumab CDRH1 | GGTFNNNAIN |
| Belimumab CDRH2 | GIIPMFGTAK YSQNFQG |
| Belimumab CDRH3 | SRDLLLFPHH ALSP |
| Belimumab CDRL1 | QGDSLRSYYA S |
| Belimumab CDRL2 | GKNNRPS |
| Belimumab CDRL3 | SSRDSSGNHW V |

The BLISS-52 (NCT00424476) and 76 (NCT00410384) were phase III randomised trials conducted to evaluate belimumab efficacy and safety throughout 52 and 76 weeks of treatment respectively. Not all SLE patients do not respond to belimumab treatment. A better response to belimumab treatment has been associated with higher baseline disease activity (SELENA-SLEDAI ≥10), anti-dsDNA positivity, low complement levels or corticosteroid treatment at baseline. The baseline levels of serum BAFF were not demonstrated as a predictor of clinical response¹³.

Belimumab is approved for the treatment of SLE administered by intravenous infusion, at a dose of 10 mg/kg at 2-week intervals for the first 3 doses and at 4-week intervals thereafter. Belimumab is also approved for the treatment of SLE administered by subcutaneous injection, at a dose of 200 mg once weekly. Belimumab formulations are described in US patent application US20180289804 A1 which is incorporated herein by reference in its entirety. Belimumab may be administered at a dose of 10 mg/kg on days 0, 14 and 28, and at 4-week intervals thereafter. Belimumab may be administered at 10 mg/kg every 2 weeks for the first three doses, and then given every 4 weeks. Dosage information for belimumab is provided in Table 6-7.

TABLE 6-7
Belimumab dosage and administration

| Route | Dose | Use/Indication |
|---|---|---|
| Intravenous | 10 mg/kg Q2W for three | FDA approved for SLE |
| | weeks, then 10 mg/kg Q4W | |
| Subcutaneous | 200 mg QW | FDA approved for SLE |

### 6.27.2 Tabalumab

Tabalumab (LY2127399) is a human IgG4 monoclonal antibody that binds both soluble and membrane-bound B-cell activating factor (BAFF). The efficacy and safety of tabalumab was assessed in two 52-week, phase III, multicentre randomized, double-blind, placebo-controlled trial in patients with moderate-to-severe SLE (ILLUMINATE-1 and ILLUMINATE-2). The primary endpoint was proportion of patients achieving SLE Responder Index 5 (SRI-5) response at week 52. In ILLUMINATE-1 (NCT01196091), the primary endpoint was not met. Key secondary efficacy endpoints (OCS sparing, time to severe flare, worst fatigue in the last 24 hours) also did not achieve statistical significance, despite pharmacodynamic evidence of tabalumab biological activity (significant decreases in anti-dsDNA, total B-cells, and immunoglobulins)³⁸. The primary endpoint was met in ILLUMINATE-2 (NCT01205438) in the higher dose group (tabalumab 120 mg every 2 weeks). However, no secondary endpoints were met, including OCS sparing³⁹. Following ILLUMINATE-1 and ILLUMINATE-2, tabalumab development was suspended given the small effect size and inability to meet other important clinical endpoints. Dose information is provided in Table 6-8.

TABLE 6-8
Tabalumab dosage and administration

| | | | Use/ |
|---|---|---|---|
| Study | Route | Dose | Indication |
| ILLUMINATE-1 | subcutaneous | loading dose (240 mg) | SLE |
| | | at week 0 and followed | |
| | | by 120 mg every two | |
| | | weeks, 120 mg every | |
| | | four weeks | |
| ILLUMINATE-1 | subcutaneous | 120 mg every four weeks | SLE |
| ILLUMINATE-2 | subcutaneous | loading dose (240 mg) | SLE |
| | | at week 0 and followed | |
| | | by 120 mg every two | |
| | | weeks, 120 mg every | |
| | | four weeks | |
| ILLUMINATE-2 | subcutaneous | 120 mg every four weeks | SLE |

### 6.28 Patient Reported Outcomes (PROs)

In particular embodiments, treatment using anifrolumab results in an MCR. In particular embodiments, treatment using anifrolumab results in a PCR.

The SF-36-v2 (acute) is a multipurpose, 36-item survey that measures 8 domains of health: physical functioning, role limitations due to physical health, bodily pain, general health perceptions, vitality, social functioning, role limitations due to emotional problems, and mental health. It yields scale scores for each of these 8 health domains, and summary measures of physical and mental health: the Physical Component Summary and Mental Component Summary.

The FACIT-F is a 13-item subject-completed questionnaire to assess the impact of fatigue over the previous 7 days. The responses range from 0 (Not at All) to 4 (Very Much). Final scores are the sum of the responses and range from 0 to 52; higher scores indicate better QoL (Yellen et al, 1997). Changes in scores >3 points are considered to be clinically meaningful.

The PtGA is a single-item question that takes into account all the ways in which illness and health conditions may affect the patient at this time. The patient should consider the previous week when answering this question. Responses range from very well to very poor on a 100 mm VAS. The physician and subject must complete the PGA and PtGA, respectively, independently of each other.

A Major Clinical Response (MCR) includes BICLA scores C or better at Week 24, maintained with no new A or B scores between Week 24-52. A Partial Clinical Response (PCR) includes a maximum of 1 BICLA score at Week 24, maintained without new A or >1 new B domain score through Week 52.

Physician Global Assessment (PGA) of Disease Activity refers to an assessment wherein a physician evaluates the status of a subject's psoriatic arthritis (PsA) by means of a visual analog scale (VAS). The subject is assessed according to how their current arthritis is. The VAS is anchored with verbal descriptors of “very good” to “very poor.”

The method may comprise measuring PROs in the subject before and after administration of anifrolumab. The PRO's may comprise the subject's Functional Assessment of Chronic Illness Therapy-Fatigue (FACIT-F), Short Form 36 Health Survey version 2 (SF-36-v2), mental component summary (MCS), and/or SF-36, physical component summary (PCS) score.

### 7 EXAMPLES

The Examples that follow are illustrative of specific embodiments of the disclosure, and various uses thereof. They are set forth for explanatory purposes only and should not be construed as limiting the scope of the disclosure in any way.

### 8 Example 1: MUSE phase IIb

Study 1013 (NCT01438489) was a Phase 2, multinational, multicenter, randomized, double-blind, placebo controlled, parallel-group study to evaluate the efficacy and safety of 2 intravenous (IV) treatment regimens in adult participants with chronic, moderately-to-severely active SLE with an inadequate response to SOC SLE. The investigational product (anifrolumab or placebo) was administered as a fixed dose every 4 weeks (28 days) for a total of 13 doses.

Study 1013 randomized 307 patients (1:1:1) and compared anifrolumab, 300 mg or 1000 mg, to placebo. The primary endpoint was a combined assessment of the SLE Responder Index (SRI-4, a composite endpoint) and the sustained reduction in OCS (<10 mg/day and ≤OCS dose at week 1, sustained for 12 weeks) measured at Week 24; a significantly higher proportion of anifrolumab 300 mg-treated patients achieved SRI-4 response and sustained OCS reduction (anifrolumab: placebo 34% vs 18%). Pre-specified analysis of disease activity measured by British Isles Lupus Assessment Group based Composite Lupus Assessment (BICLA) was 53% for anifrolumab and 25% placebo at Week 52. The dose-response modelling and benefit-risk profile supported the evaluation of the 300 mg dose in the subsequent studies.

Study 1145 (NCT01753193) was an open-label extension (OLE) for subjects completing study 1013. In particular, Study 1145 was a 3-year, multinational OLE in adults with moderate to severe SLE (per ACR classification criteria, assessed in Study 1013) who completed randomized treatment with anifrolumab 1000 or 300 mg or placebo in Study 1013 to Day 337 with follow-up to Day 422. All patients in Study 1145 initially received IV anifrolumab 1000 mg every 4 weeks (Q4W). After data from Study 1013 showed the 300-mg dose had a better benefit/risk profile, the dosage in Study 1145 was amended to 300 mg Q4W. Patients received anifrolumab Q4W over 156 weeks with 85 days of follow-up. The primary objective was to evaluate long-term safety/tolerability. Efficacy, pharmacodynamics, and health-related quality of life (HRQoL) were exploratory objectives. Safety was assessed at every visit; SLEDAI-2K and SLICC Damage Index were measured every 3 and 6 months, respectively.

Type I IFN inducible signature in whole blood was assessed by a 21-gene assay to be used as a PD marker to follow the biologic effect of anifrolumab on its target throughout the study. Whole blood was collected in order to evaluate the mRNA expression levels of 21 type I IFN-inducible genes (Table 6-2).

### 9 Example 2: TULIP I and TULIP II (ClinicalTrial.gov Identifier: NCT02446912 and NCT02446899)

The purpose of these study was to evaluate the efficacy and safety of an intravenous treatment regimen of two doses of anifrolumab versus placebo in adult subjects with moderately to severely active, autoantibody-positive systemic lupus erythematosus (SLE). The studies were Phase 3, multi-centre, multinational, randomized, double-blind, placebo-controlled studies to evaluate the efficacy and safety of an intravenous treatment regimen of two doses of anifrolumab versus placebo in subjects with moderately to severely active, autoantibody-positive systemic lupus erythematosus (SLE) while receiving standard of care (SOC) treatment.

TULIP I and II were similar in design, the primary endpoint was improvement in disease activity evaluated at 52 weeks, measured by SRI-4 and BICLA, respectively. The common secondary efficacy endpoints included in both studies were the maintenance of OCS reduction, improvement in cutaneous SLE activity measured by Cutaneous Lupus Erythematosus Disease Area and Severity Index (CLASI), and annualized flare rate. An assessment of improvement in joint activity was included as a secondary endpoint in TULIP II. Both studies evaluated the efficacy of anifrolumab 300 mg versus placebo; a dose of 150 mg was also evaluated for dose-response in TULIP I.

Patient demographics were generally similar in both trials; 92% and 93% were female, 71% and 60% were White, 14% and 12% were Black/African American, and 5% and 17% were Asian, in TULIP I and II respectively. In both trials, 72% of patients had high disease activity (SLEDAI-2K score ≥10). In TULIP I and II respectively, 48% and 49% had severe disease (BILAG A) in at least 1 organ system and 46% and 47% of patients had moderate disease (BILAG B) in at least 2 organ systems. The most commonly affected organ systems (BILAG A or B at baseline) were the mucocutaneous (TULIP I: 87%, TULIP II: 85%) and musculoskeletal (TULIP II: 89%, TULIP II: 88%) systems; 7.4% and 8.8% of patients had cardiorespiratory, and 7.9% and 7.5% had renal manifestations at baseline, in TULIP I and II respectively.

In TULIP I and II, 90% of patients (both trials) were seropositive for anti-nuclear antibodies (ANA), and 45% and 44% for anti-double-stranded DNA (anti-dsDNA) antibodies, 34% and 40% had low C3, and 21% and 26% had low C4. The majority of patients were classified as interferon gene signature test-high at baseline (TULIP 1: 82%, TULIP II: 83%). Baseline concomitant standard therapy medications included oral corticosteroids (TULIP I: 83%, TULIP II: 81%), antimalarials (TULIP 1: 73%, TULIP II: 70%) and immunosuppressants (TULIP 1: 47%, TULIP: II: 48%; including azathioprine, methotrexate, mycophenolate and mizoribine). For those patients taking OCS (prednisone or equivalent) at baseline, the mean daily dose was 12.3 mg in TULIP I and 10.7 mg in TULIP II. During Weeks 8-40, patients with a baseline OCS ≥10 mg/day were required to taper their OCS dose to ≤7.5 mg/day, unless there was worsening of disease activity.

Randomization was stratified by disease severity (SLEDAI-2K score at baseline, <10 vs ≥10 points), OCS dose on Day 1 (<10 mg/day vs ≥10 mg/day prednisone or equivalent) and interferon gene signature test results (high vs low).

### 10 Example 3: Results of TULIP I (ClinicalTrial.gov Identifier: NCT02446912)

TULIP I randomized 457 patients to receive anifrolumab 150 mg, 300 mg or placebo (1:2:2). The primary endpoint, SRI 4 response, was defined as meeting each of the following criteria at Week 52 compared with baseline:
Reduction from baseline of ≥4 points in the SLEDAI-2K;
No new organ system affected as defined by 1 or more BILAG A or 2 or more BILAG B items compared to baseline;
No worsening from baseline in the patients' lupus disease activity defined by an increase ≥0.30 points on a 3-point PGA visual analogue scale (VAS);
No discontinuation of treatment;
No use of restricted medication beyond the protocol-allowed threshold.

For the primary endpoint (SRI-4 at Week 52), treatment with anifrolumab did not result in statistically significant improvements over placebo (p-value=0.455). The secondary endpoints were not formally tested; however, clinically meaningful improvements in BICLA response, sustained OCS dose reduction, CLASI response, flare rate and joint response were observed for patients receiving anifrolumab 300 mg compared to those receiving placebo. The BICLA responder rate was 47% (85/180) for anifrolumab 300 mg versus 30% (55/184) for placebo (difference 17%, 95% CI 7.2, 26.8, nominal p-value <0.001).

### 11 Example 4: TULIP I and TULIP II (ClinicalTrial.gov Identifier: NCT02446899)

TULIP II randomized 362 patients (1:1) that receive anifrolumab 300 mg or placebo. The primary endpoint, BICLA response at Week 52, was defined as improvement in all organ domains with moderate or severe activity at baseline:
Reduction of all baseline BILAG A to B/C/D and baseline BILAG B to C/D, and no BILAG worsening in other organ systems, as defined by ≥1 new BILAG A or ≥2 new BILAG B;
No worsening from baseline in SLEDAI-2K, where worsening is as defined as an increase from baseline of >0 points in SLEDAI-2K;
No worsening from baseline in patients' lupus disease activity, where worsening is defined by an increase ≥0.30 points on a 3-point PGA VAS;
No discontinuation of treatment;
No use of restricted medication beyond the protocol-allowed threshold.

The primary endpoint was met; anifrolumab 300 mg demonstrated statistically significant and clinically meaningful efficacy in overall disease activity compared with placebo. Greater improvements in all components of the BICLA composite endpoint were observed for anifrolumab compared to placebo (Table 11-1).

TABLE 11-1
BICLA response rate at Week 52

| | |
|---|---|
| Anifrolumab- | |
| xxxx 300 mg | Placebo |
| (N = 180) | (N = 182) |

BICLA response rate

| | | | | |
|---|---|---|---|---|
| Responder, n (%) | 86 | (48) | 57 | (32) |

| | |
|---|---|
| Difference in Response Rates (95% CI) | 16% (6.3, 26.3) |
| p-value (2-sided) | 0.0013 |

Components of BICLA response

| | | | | |
|---|---|---|---|---|
| BILAG improvement, n (%) * | 88 | (49) | 59 | (32) |
| No worsening of SLEDAI-2K, n (%) * | 122 | (68) | 94 | (52) |
| No worsening of PGA, n (%) * | 122 | (68) | 95 | (52) |
| No discontinuation of treatment, n (%) | 153 | (85) | 130 | (71) |
| No use of restricted medication beyond | 144 | (80) | 123 | (68) |
| protocol allowed threshold, n (%) | | | | |

All patients received standard therapy.

Clinical meaningful difference in BICLA response rate were observed as early as Week 8. Compared to placebo, the clinical benefit of anifrolumab was maintained through Week 52 (FIG. 1B).

Treatment with anifrolumab reduced the time to the first visit at which BICLA response was attained and subsequently sustained up to, and including, Week 52. At any time during the study, patients treated with anifrolumab were 55% more likely to achieve a sustained BICLA response, relative to patients receiving placebo (hazard ratio=1.55, 95% CI 1.11, 2.18). Separation between the treatment arms began at approximately Week 4 (FIG. 1C).

The treatment effect of anifrolumab relative to placebo was consistent across subgroups (by age, gender, race, ethnicity, disease severity [SLEDAI-2K at baseline], and baseline OCS use). Pre-specified analysis of disease activity measured by SRI-4 was consistent with the response measured by BICLA (SRI-4 responder rate; anifrolumab 56% vs placebo 37%; difference 18% [95% CI 8.1, 28.3]).

### 11.1 Effect on Concomitant Steroid Treatment

In the 47% of patients with a baseline OCS use ≥10 mg/day, anifrolumab demonstrated a statistically significant and clinically meaningful reduction in OCS use, of at least 25% to ≤7.5 mg/day at Week 40 maintained through to Week 52 (p-value=0.004); 52% (45/87) of patients in the anifrolumab group versus 30% (25/83) in the placebo achieved this level of steroid reduction (difference 21% [95% CI 6.8, 35.7]). In patients with a baseline OCS use ≥10 mg/day, the median (min, max) cumulative OCS dose at Week 52 was 3197 mg (309, 13265) compared to 3640 mg (1745, 10920) for the anifrolumab and placebo groups, respectively.

### 11.2 Effect on Cutaneous SLE Activity

In patients with moderate to severe skin disease at baseline (CLASI activity score ≥10; n=89), anifrolumab demonstrated statistically significant and clinically meaningful improvements in cutaneous lupus activity (CLASI response: defined as, at least 50% reduction in CLASI activity score compared to baseline) at Week 12 (responder rate 49% [24/49] and 25% [10/40] for the anifrolumab and placebo group, respectively; observed difference 24% [95% CI 4.3, 43.6], p-value=0.017). Compared to placebo, the treatment benefit of anifrolumab was maintained through Week 52. Patients with moderate to severe skin disease at baseline who received anifrolumab were 55% more likely to achieve a sustained CLASI response (defined as a CLASI response attained at any time during the study and subsequently sustained up to, and including Week 52) relative to patients receiving placebo (hazard ratio=1.55, 95% CI 0.87, 2.85).

### 11.3 Effect on SLE Flares

Disease flare was defined as severe disease activity (BILAG A) in one or more new organ system, or moderate disease activity (BILAG B) in 2 or more new organ systems compared to the previous visit. Anifrolumab led to a clinically meaningful 33% reduction of the annual flare rate versus placebo (annualized rate 0.43 and 0.64 for the anifrolumab and placebo group, respectively; rate ratio 0.67 [95% CI 0.48, 0.94], p-value=0.020); this difference was not statistically significant following adjustment for multiple comparisons. In TULIP II, 69% (124/180) of patients receiving anifrolumab experienced no SLE flares compared to 58% (105/182) of patients receiving placebo, during the 52-week treatment period. The time to first flare was longer in the anifrolumab group, at any time during the study patients had a 35% lower risk of experiencing a first flare relative to patients receiving placebo (hazard ratio=0.65 [95% CI 0.46, 0.91]).

### 11.4 Effect on Joint Activity

At baseline 44% of patients had ≥6 swollen and 26 tender joints. Response was defined as ≥50% improvement in swollen/tender joint count at Week 52. There was no notable difference in joint response between treatment groups (response rate 42% [30/71] and 38% [34/90] for the anifrolumab and placebo group, observed difference 4.7% [95% CI-10.6, 20.0], p-value=0.547).

### 12 Example 5: Efficacy in Patient Subgroups

### 12.1 Objective

To compare BICLA responses to anifrolumab vs placebo through Week 52 in protocol-defined subgroups of patients in TULIP-1 and TULIP-2 and across pooled TULIP-1 and TULIP-2 data. Baseline characteristics are shown in Table 12-1 and Table 12-2.

### 12.2 Results

There was a robust BICLA response rates observed at Week 52 with anifrolumab across prespecified subgroups in TULIP-1, TULIP-2, and pooled TULIP data. There was no substantive impact on effect size of demographics (FIG. 43), baseline disease activity (FIG. 44), baseline OCS dosage (FIG. 45), baseline Type I IFNGS test status (FIG. 46A). The response rate to anifrolumab was similar in IFNGS test (4-gene)-high and-low patients (FIG. 46B).

TABLE 12-1
Baseline Patient Demographics

| | | Pooled TULIP^{a} | | | |
|---|---|---|---|---|---|
| | | | | Anifrolumab | |
| Baseline | | Placebo | | 300 mg | |
| patient demographics | | (n = 366) | | (n = 360) | |
| Age, mean (SD), years | | 41.0 | (11.9) | 42.6 | (12.0) |
| Female, n (%) | | 341 | (93.2) | 333 | (92.5) |
| BMI, n (%) | ≤28 kg/m² | 223 | (60.9) | 205 | (56.9) |
| | >28 kg/m² | 143 | (39.1) | 155 | (43.1) |
| Race, n (%) | White | 244 | (66.7) | 235 | (65.3) |
| | Black/African American | 48 | (13.1) | 46 | (12.8) |
| | Asian | 35 | (9.6) | 41 | (11.4) |
| | Other | 31 | (8.5) | 30 | (8.3) |
| Geographic | Asia-Pacific | 32 | (8.7) | 38 | (10.6) |
| region, n (%) | Europe | 122 | (33.3) | 115 | (31.9) |
| | Latin America | 57 | (15.6) | 59 | (16.4) |
| | USA/Canada | 140 | (38.3) | 139 | (38.6) |
| | Rest of world | 15 | (4.1) | 9 | (2.5) |

TABLE 12-2
Baseline Disease Characteristics

| | | Pooled TULIP^{a} | | | |
|---|---|---|---|---|---|
| | | Placebo | | Anifrolumab | |
| Baseline disease characteristics | | (n = 366) | | 300 mg (n = 360) | |
| SLEDAI-2K | Mean (SD) | 11.3 | (3.6) | 11.4 | (3.9) |
| (screening) | ≥10, n (%) | 260 | (71.0) | 251 | (69.7) |
| OCS dosage, | <10 mg/day | 181 | (49.5) | 170 | (47.2) |
| n (%) | ≥10 mg/day | 185 | (50.5) | 190 | (52.8) |
| Type I IFNGS | High | 302 | (82.5) | 298 | (82.8) |
| status,^{d} n (%) | Low | 64 | (17.5) | 62 | (17.2) |

### 13 Example 6: A Quantitative Systems Pharmacology Modelling of Anifrolumab in Treatment of SLE

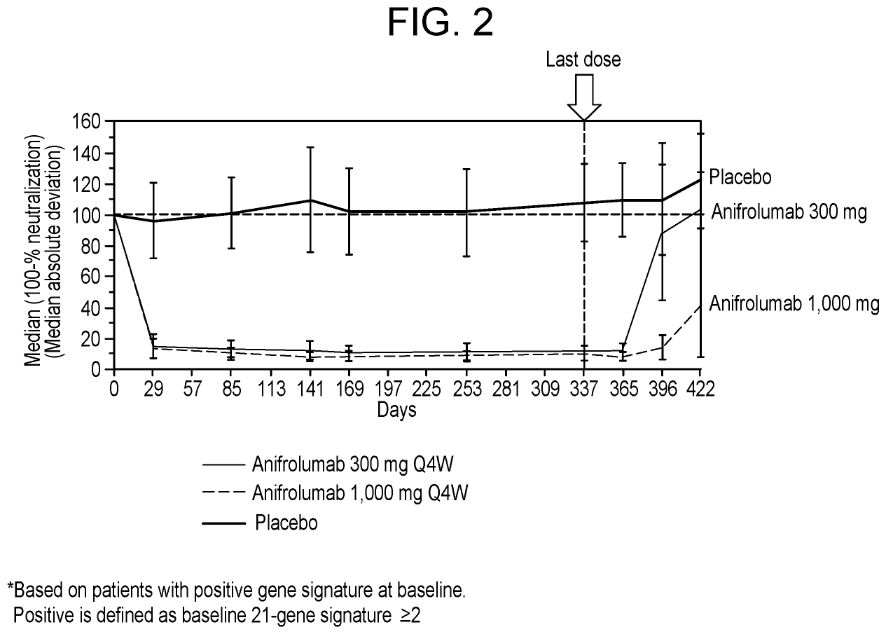

As has been previously described, in the MUSE Phase IIb study, expression of type I IFN-inducible genes in whole blood using a 21-gene panel⁶ (pharmacodynamic [PD] marker) decreased following anifrolumab administration for all dose groups in subjects with a baseline positive type I IFN signature in whole blood¹⁴. Both the 300 mg and 1000 mg anifrolumab dose achieved and maintained 82 to 90% neutralization of the gene signature. In the placebo group no neutralization of the gene signature (>6%) was observed at any time point. Anifrolumab thus neutralizes the 21-gene type I IFN PD signature in the whole blood of SLE patients (FIG. 2).

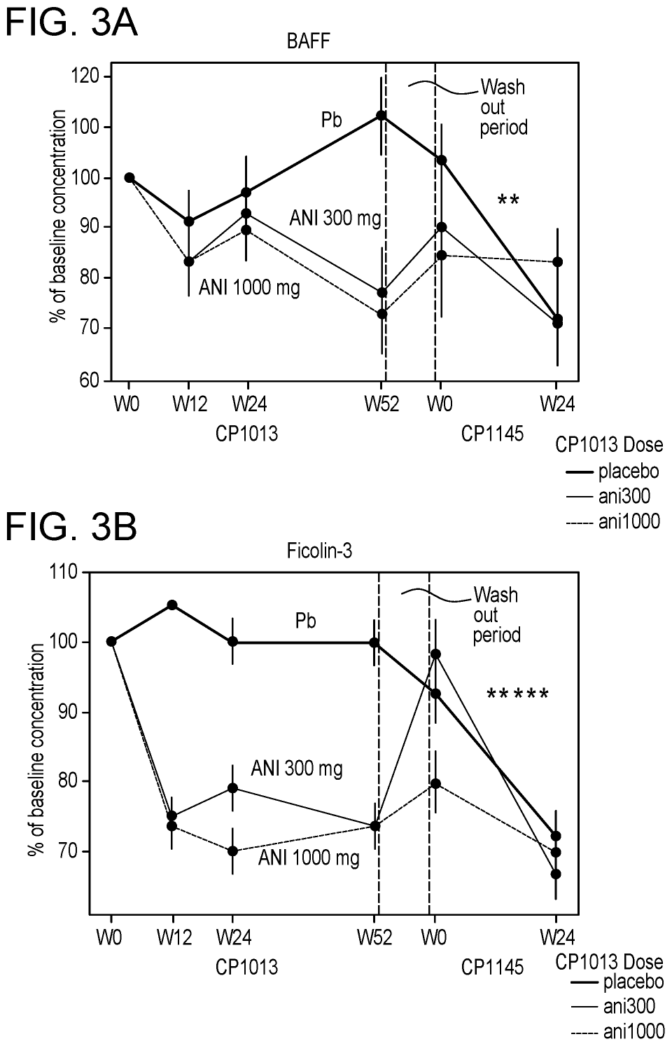

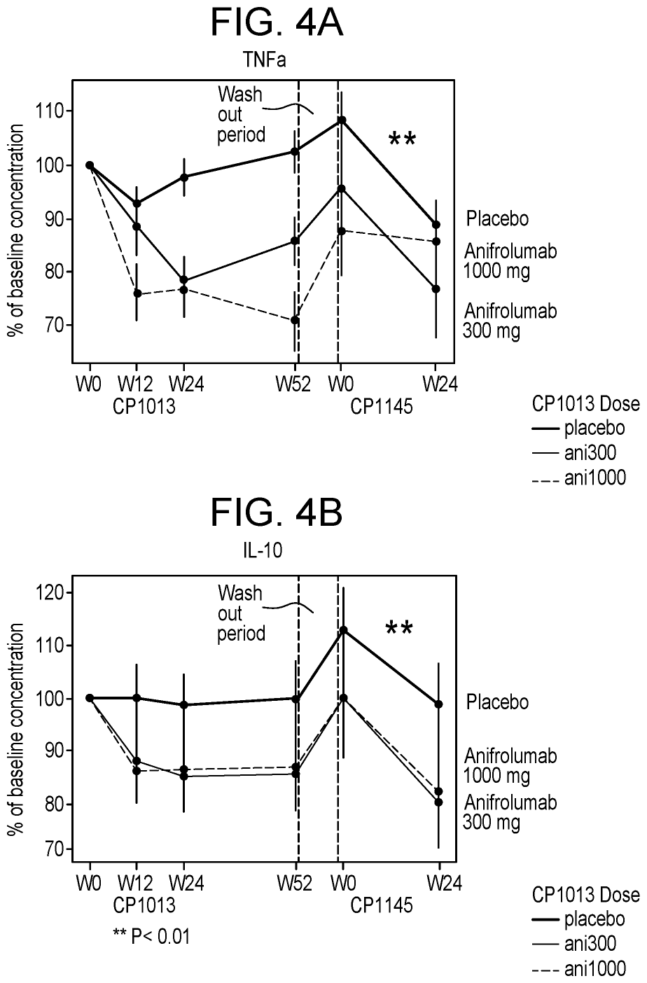

Serum samples were taken from patients in the MUSE trial pre-and post-treatment for placebo and anifrolumab treatment groups. The serum samples were analyzed for cytokine expression using Luminex® or ultrasensitive Simoa immunoassay, according to the standard protocols. The LLOQ of the Simoa assay is 0.037 pg/ml. Anifrolumab was shown to induce alterations in many serum proteins levels, indicating that anifrolumab has effects on multiple cell types (Table 13-1) (FIG. 3 and FIG. 4).

TABLE 13-1
Anifrolumab induced changes in serum protein levels

| | | Alteration in |
|---|---|---|
| | | level elicited |
| | | by Anifrolumab |
| Protein | Function/outcome | relative to placebo |
| TNSF13B (BAFF) | B cell differentiation/ | Decrease |
| | survival | (FIG. 3A) |
| CXCL13 (BLC) | B cell migration | Decrease |
| IgE | B cell differentiation/ | Decrease |
| | isotype switch | |
| CXCL10 (IP10) | T cell activation/ | Decrease |
| | migration | |
| CXCL11 (ITAC) | T cell activation/ | Decrease |
| | migration | |
| CCL2 (MCP-1) | Immune cell migration | Decrease |
| CCL8 (MCP-2) | Immune cell migration | Decrease |
| IL-1R2 (decoy | Inhibitor of IL-1 | Increase |
| R IL-1) | | |
| TNFRSF10C | Inhibitor of apoptosis | Increase |
| (TRAILR3) | | |
| Ficolin 3 | Activation of lectin | Decrease |
| | complement pathway | (FIG. 3B) |
| VCAM1 | Endothelial activation | Decrease |
| Von Willebrand | Endothelial activation | Decrease |
| Factor | | |
| Angiopoietin 2 | Endothelial activation | Decrease |
| Ferritin | Acute phase reactant | Decrease |
| Progranulin | Cofactor of TLR-9 | Decrease |
| | signaling | |

Anifrolumab was found to induce long-term downregulation of both IL-10 and TNF-alpha (FIG. 4A and FIG. 4B).

### 14 Example 7: Efficacy of Anifrolumab in Patients With Low or High IL-10 at Baseline

### 14.1 IL-10 and SLE Disease Severity

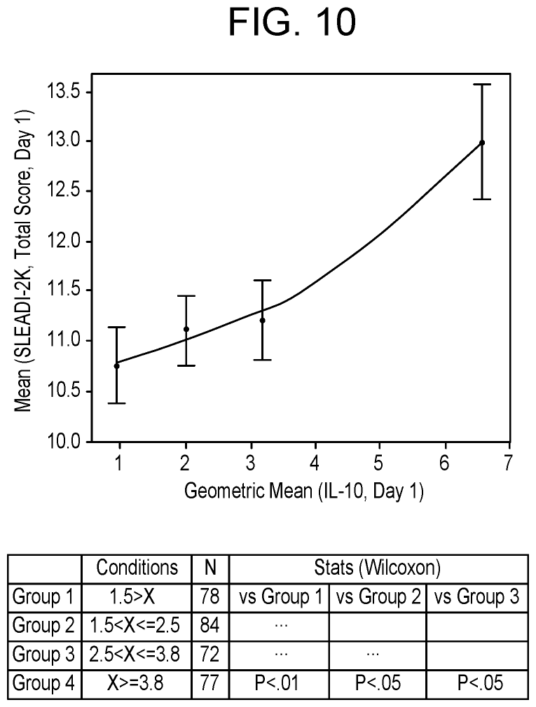

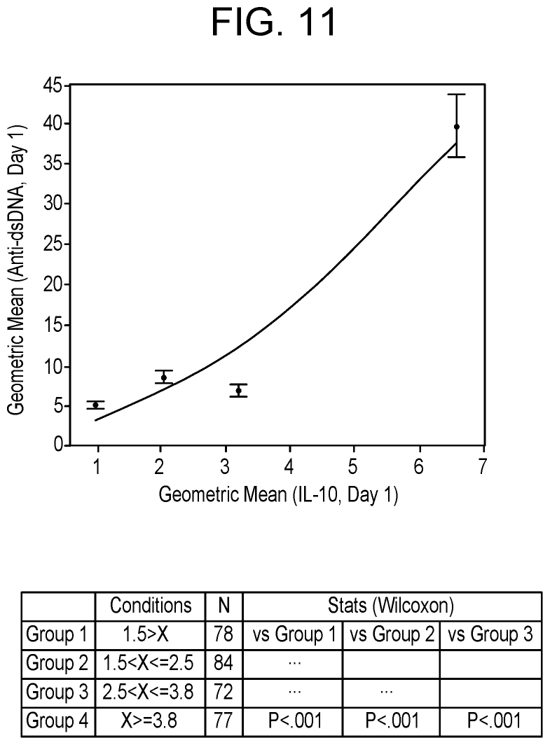

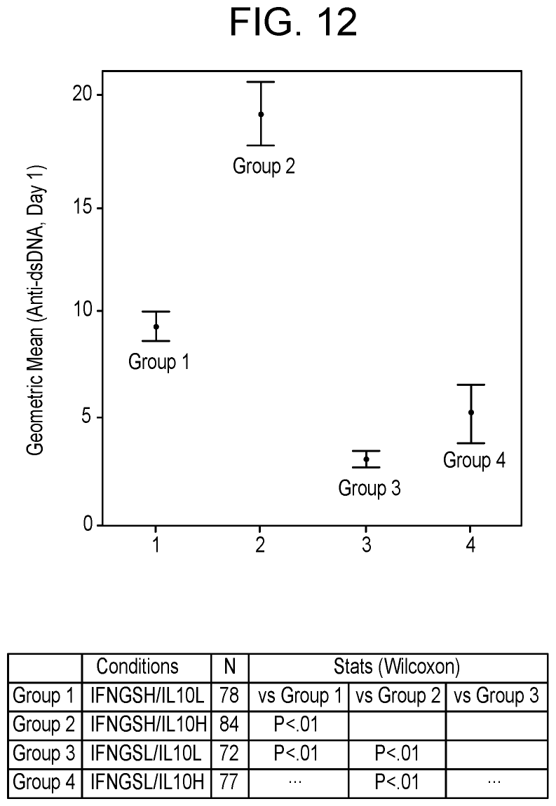

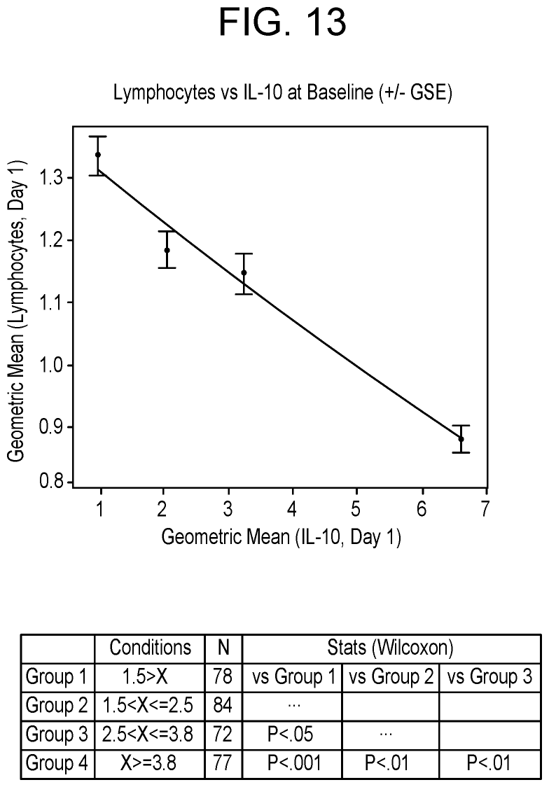

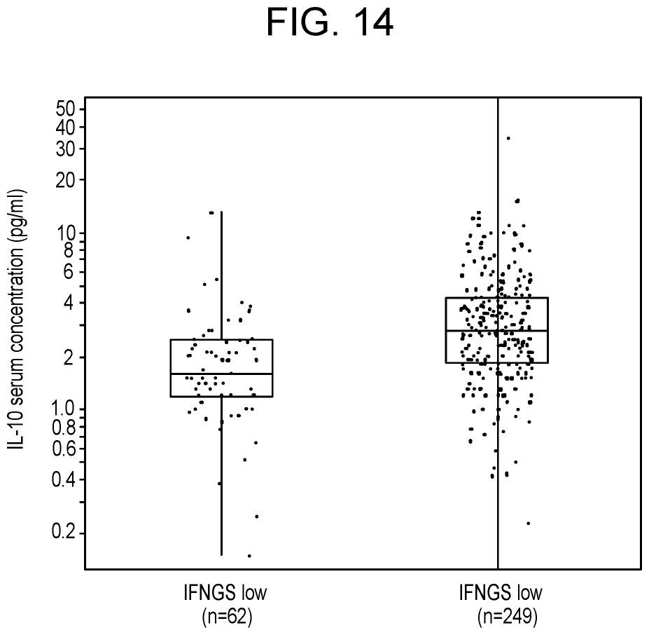

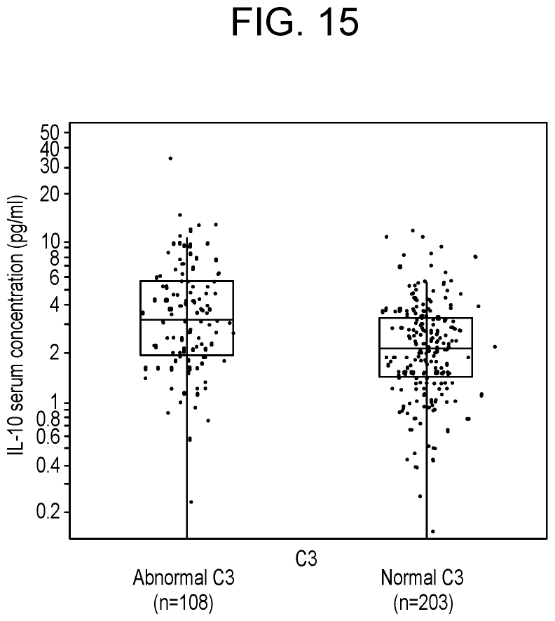

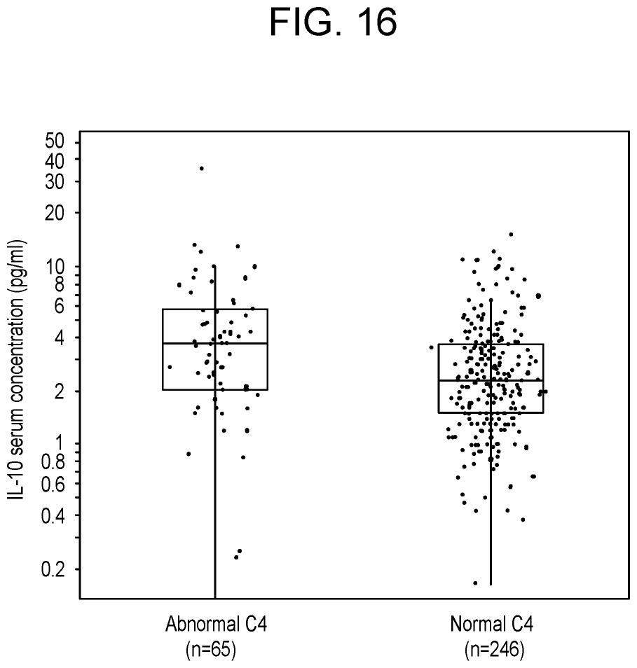

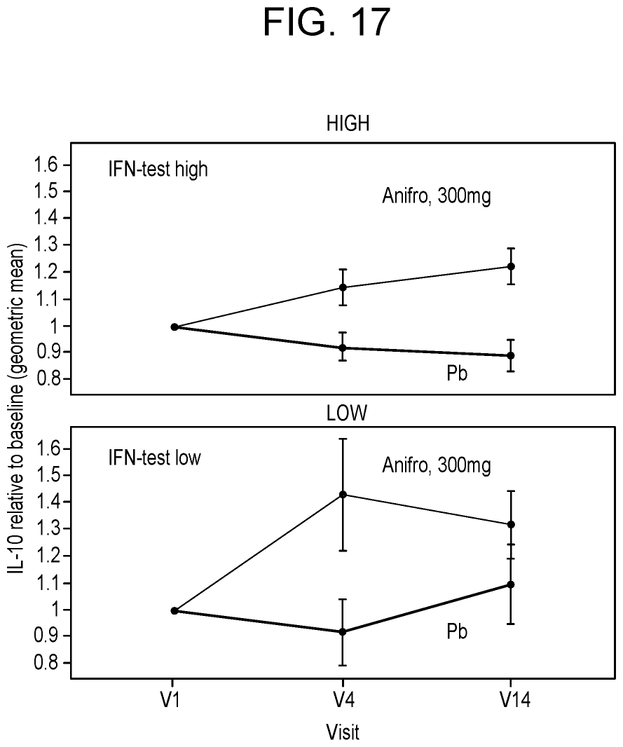

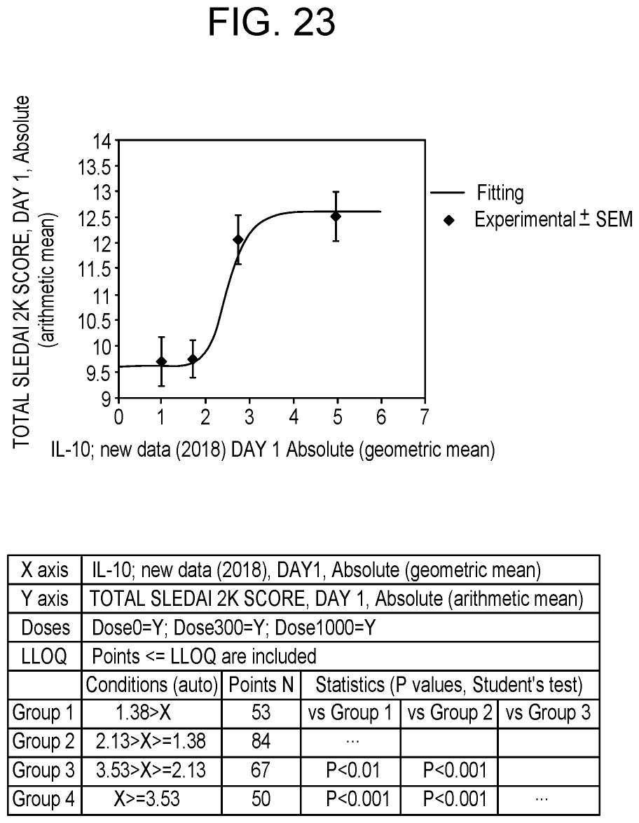

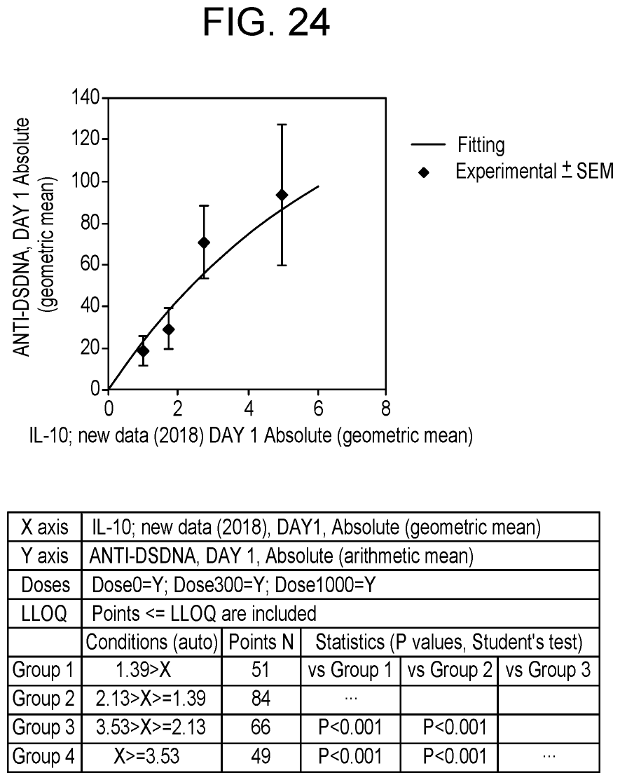

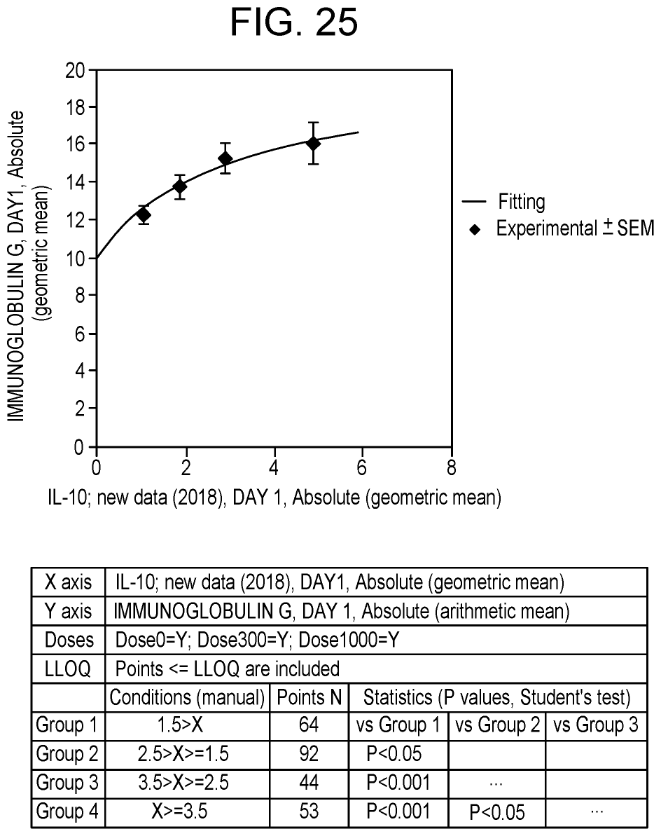

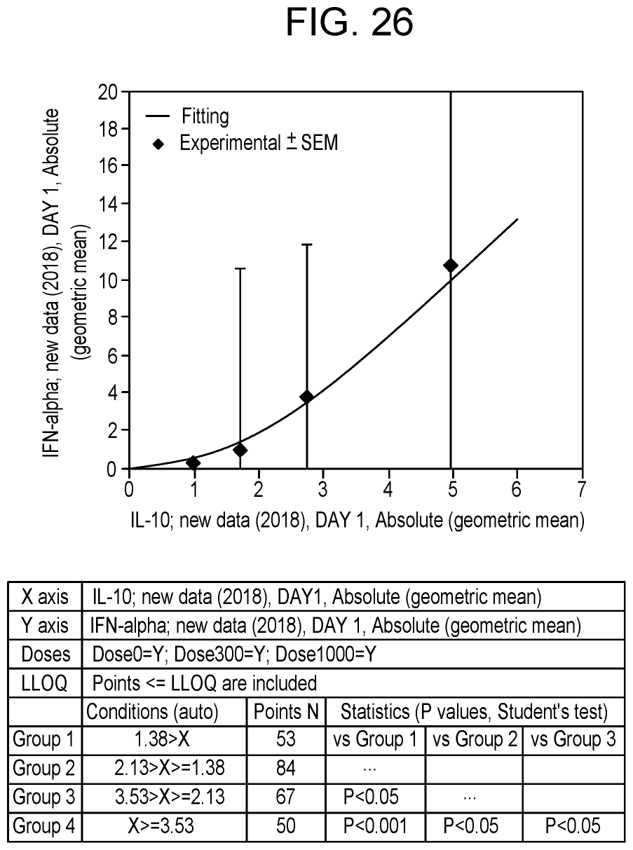

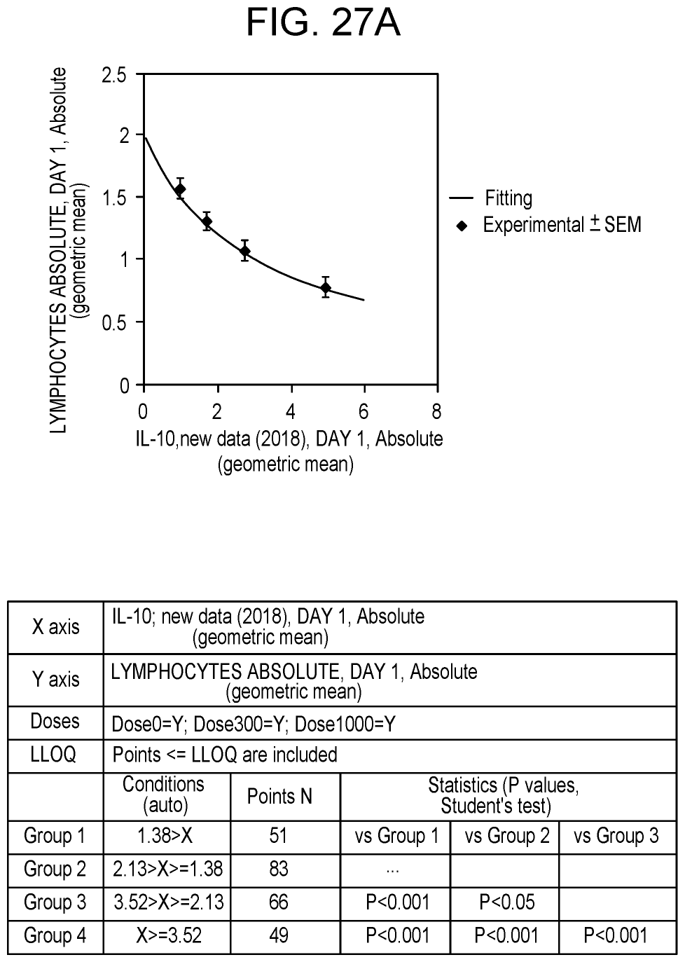

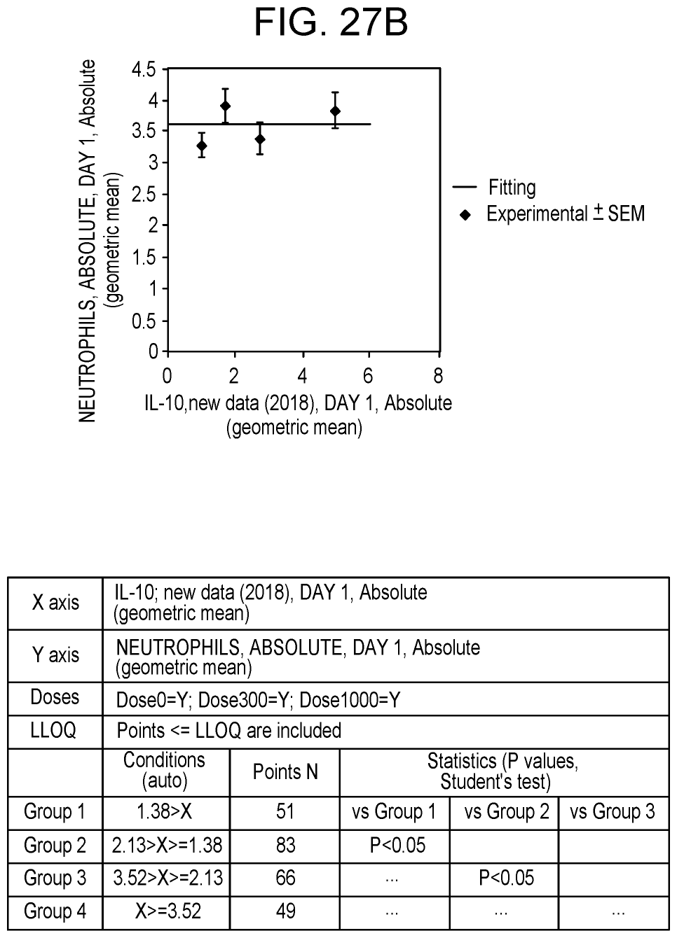

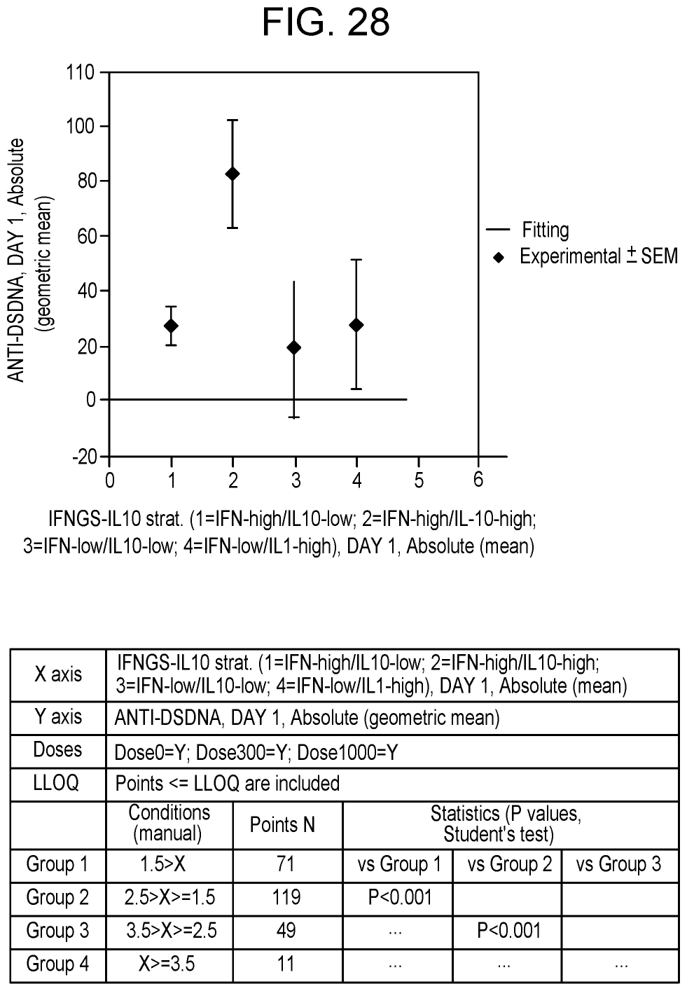

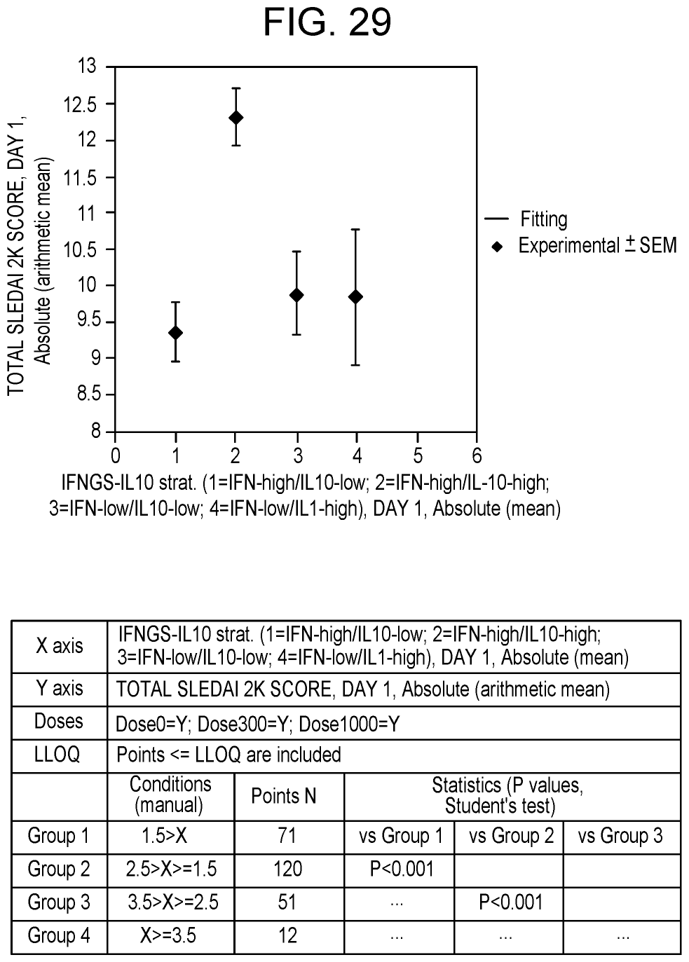

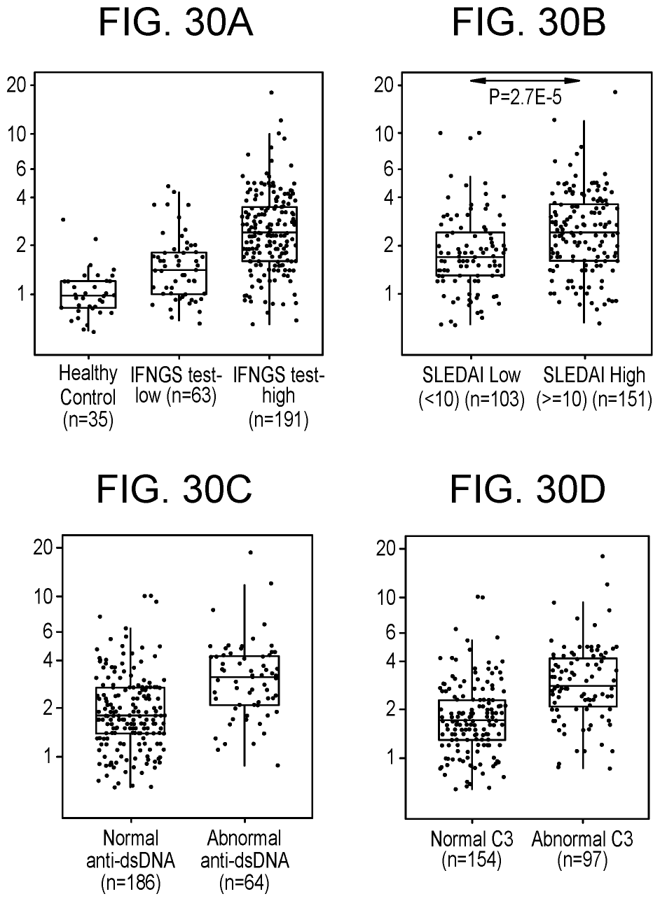

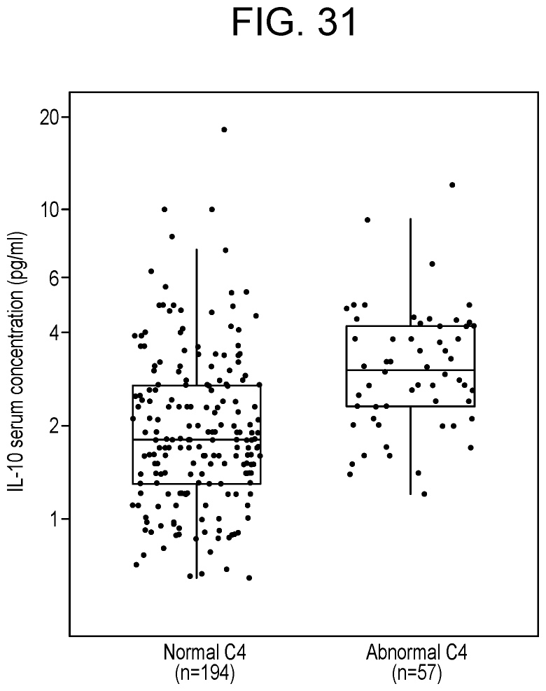

Baseline IL-10 was correlated with baseline SLEDAI 2K total score (FIG. 10, FIG. 23, FIG. 29, FIG. 30B) and the level of anti-dsDNA and auto-antibodies (FIG. 11, FIG. 24, FIG. 25, FIG. 28, FIG. 30C). Anti-dsDNA levels were highest in IFN-high and IL-10 low patients (FIG. 12). There was a negative association of IL-10 and blood lymphocyte levels in SLE patients (FIG. 13, FIG. 27A) but not correlated with neutrophil levels (FIG. 27B Error! Reference source not found.). IL-10 high SLE patient may therefore represent a sub-group of patients that do not respond to treatment with belimumab, and at least partly explain the reason for non-response to belimumab treatment in SLE patients¹⁵. IL-10 levels were also higher in IFN-high patients (FIG. 14, FIG. 30A) and patients with abnormal C3 and C4 (FIG. 15, FIG. 16, FIG. 30D, FIG. 31). Complement levels were classified as abnormal (C3 <0.9 g L⁻¹; C4 <0.1 g L⁻¹) or normal (C3 ≥0.9 g L⁻¹; C4 ≥0.1 g L⁻¹) and were measured in a central laboratory. The median fold difference between IFNGS test-high and healthy controls was 2.5. High IL-10 at baseline was also associated with a high level of type I IFN (IFN1, IFN-α) (FIG. 26).

TABLE 14-1
IL-10 patient groups

| | IL-10 concentration |
|---|---|
| Group | (X) (pg/ml) |
| 1 | 1.5 > X |
| 2 | 1.5 < X <= 2.5 |
| 3 | 2.5 < X <= 3.8 |
| 4 | X >= 3.8 |

### 14.2 IL-10 and Efficacy of Type I IFN Inhibition

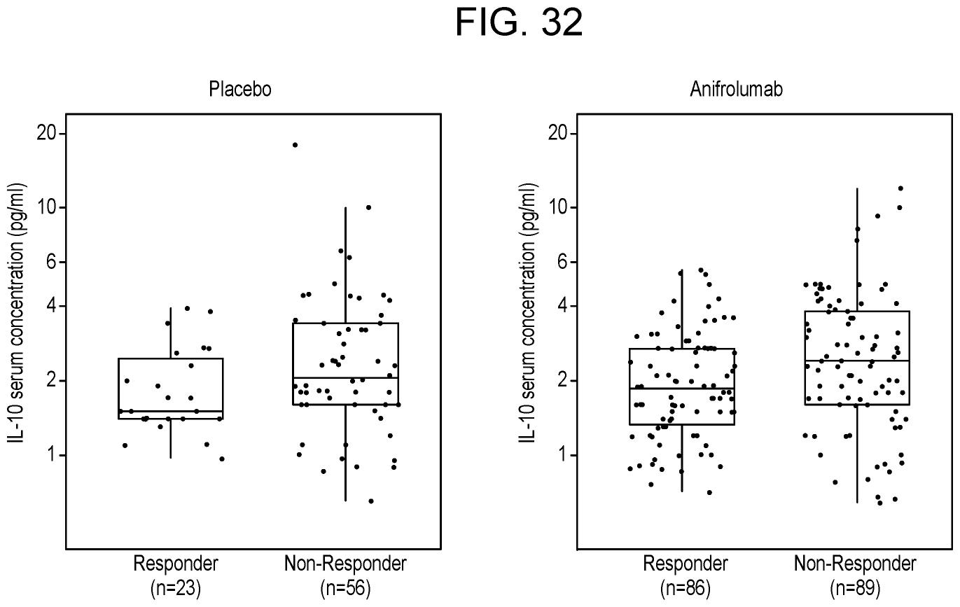

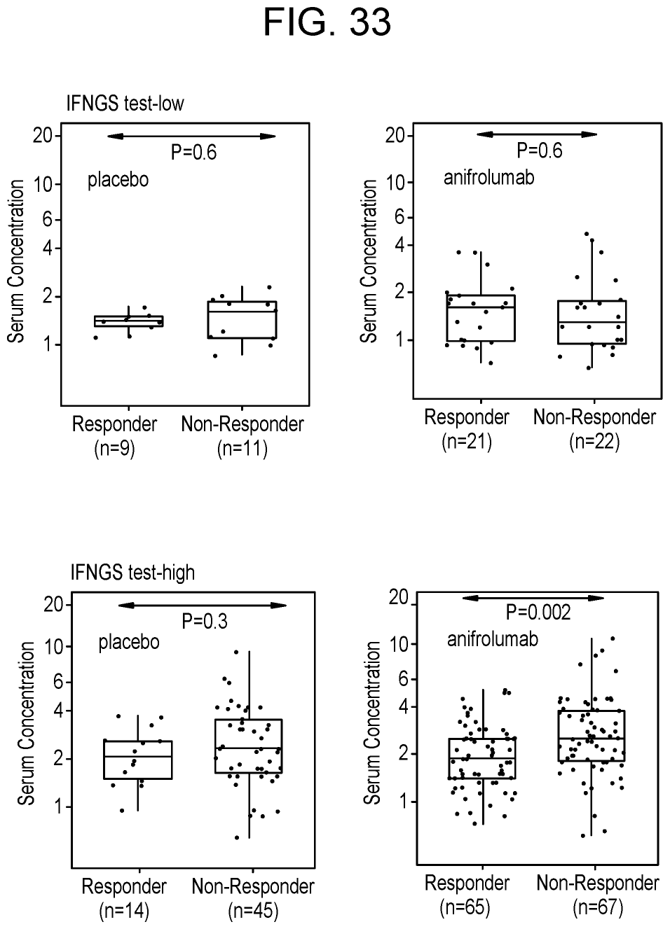

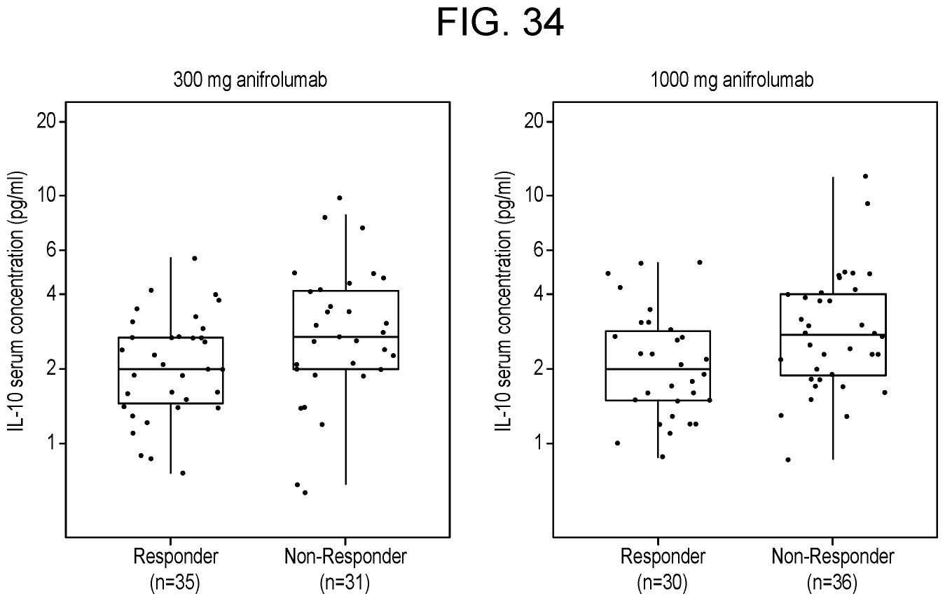

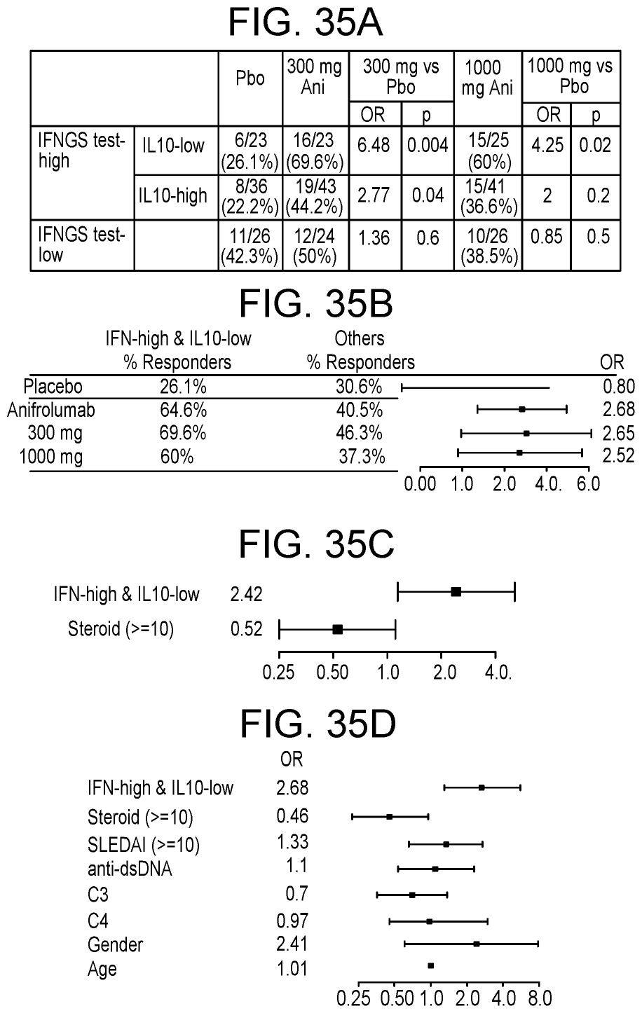

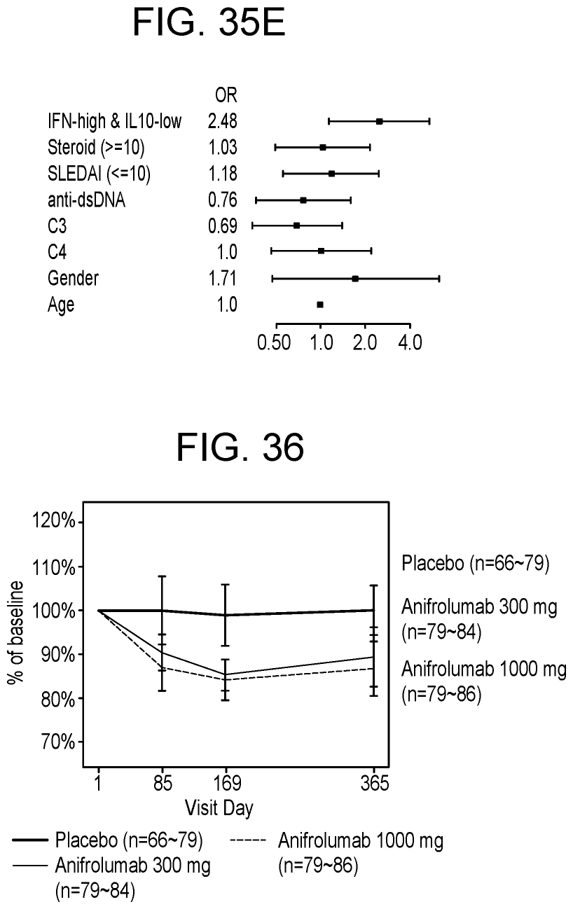

Surprisingly, baseline IL10 level was associated with clinical response at day 365 after anifrolumab treatment (FIG. 32). IFNGS test-high and IL-10 patients are the best responders to anifrolumab treatment (FIG. 33, FIG. 35B). The same effect can be seen following administration with either 300 mg or 1000 mg anifrolumab (FIG. 34). IFNGS test-high patients with IL10 lower than median (2 pg/mL) showed much higher percentage of day 365 responders after 300 mg or 1000 mg anifrolumab treatment than placebo treatment (FIG. 35A).

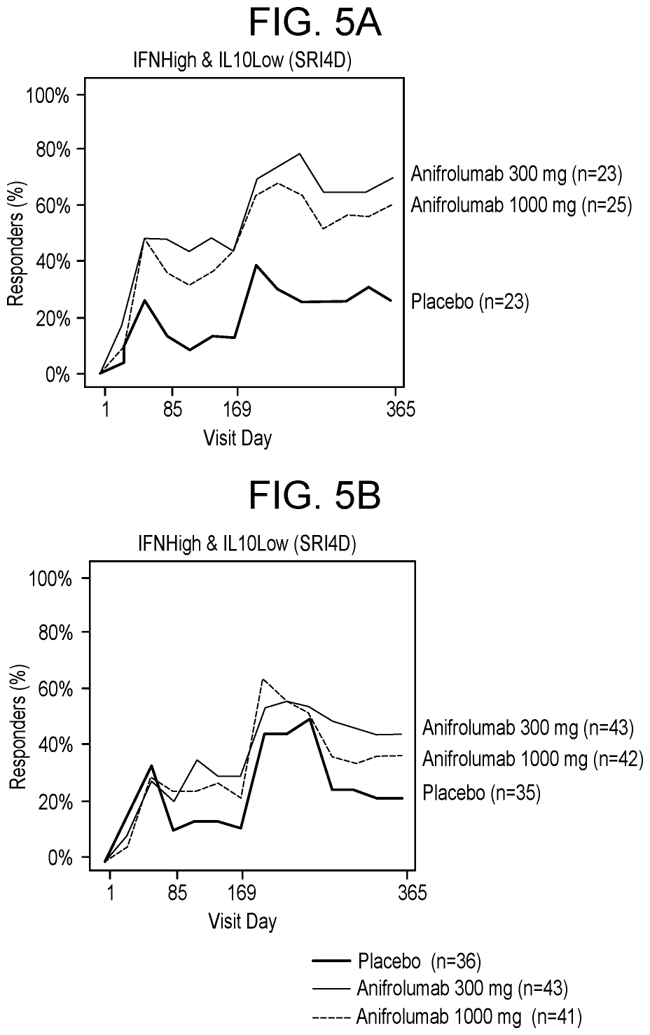

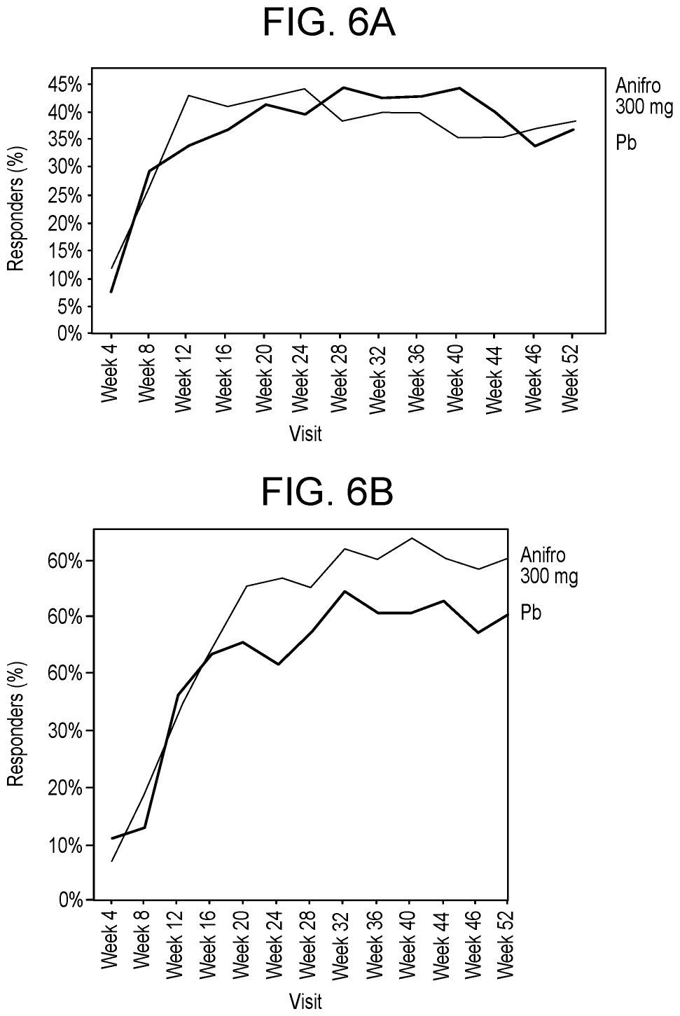

Using an IL-10 low cut-off of less than 2 pg/ml IL-10, a higher response rate (SRI4 with steroid tapering compared to placebo) was observed after administration of anifrolumab was observed in subjects with a IFNGS test-high and IL-10 low profile compared to placebo (FIG. 5A). The response rate in IFNGS test-high and IL-10-high patients (2 pg/ml IL-10 or more) was comparable to placebo (FIG. 4B). The high SRI(4) response rate in IFN-high and IL-10 low patients compared to IFN-high and IL-10 high patients data were confirmed in the TULIP I study (FIG. 6).

The present inventors thus demonstrate that IFNGS/IL10 and steroid usage are significantly predictive factors of SRI4 response status after anifrolumab treatments (FIG. 35C). Furthermore, multiple regression analysis demonstrated that IFN-high & IL10-low patients had significantly higher response rates than other patients after adjustment of steroid usage (FIG. 35D). Importantly, SLEDAI, anti-dsDNA, C3, C4, gender and age were not significant predictor for SRI4 response without steroid tapering in anifrolumab-treated patients (FIG. 35E Error! Reference source not found.).

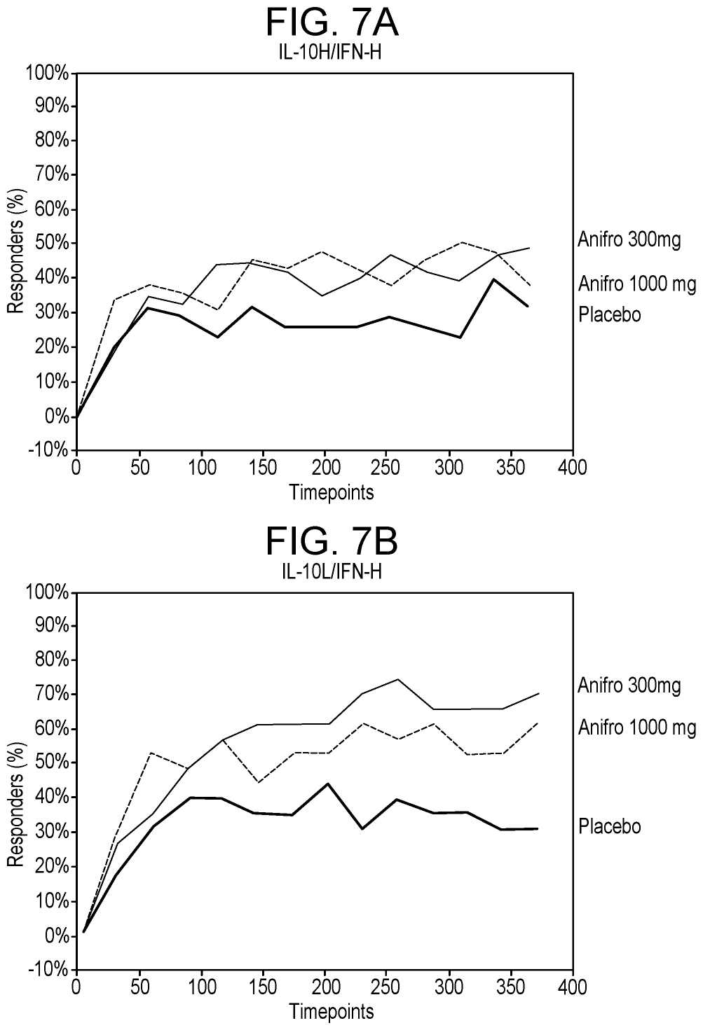

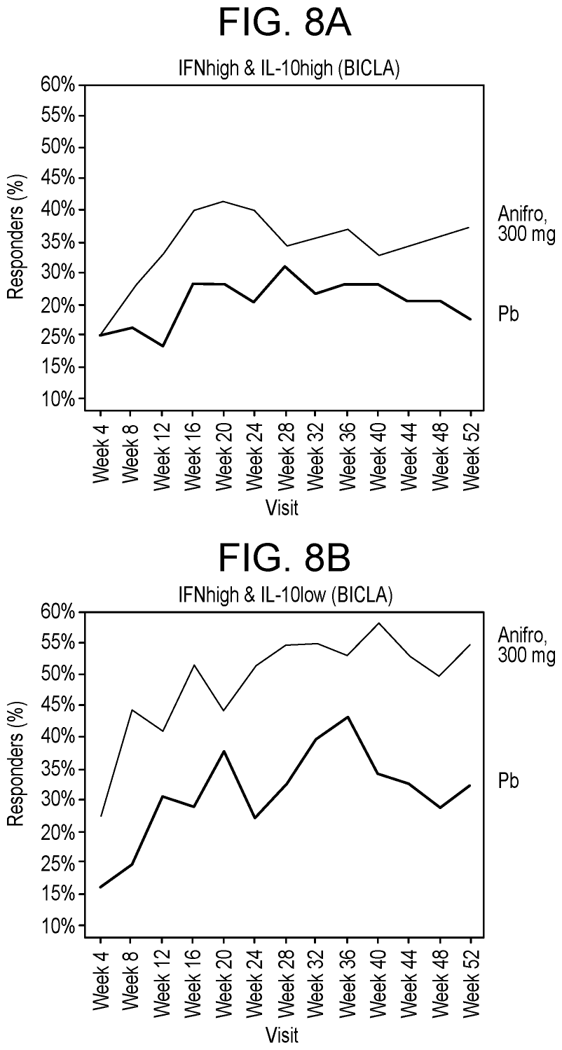

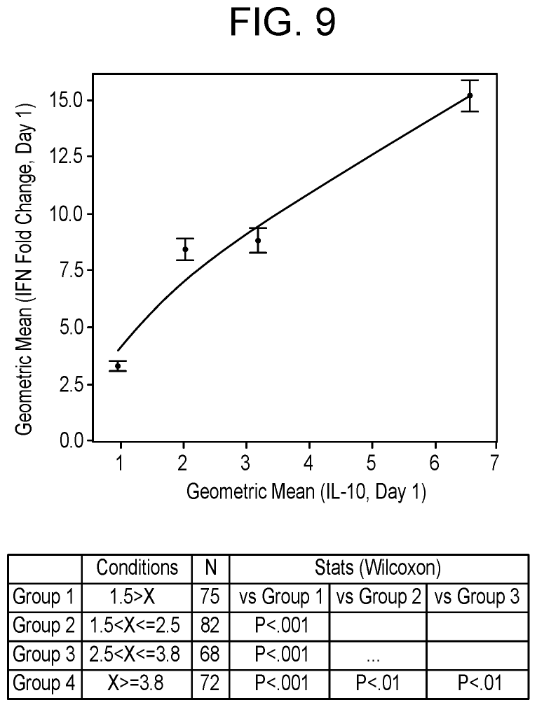

These results were confirmed in TULIP I. The delta of the 300 mg group from placebo was double the level seen in the IL-10H (FIG. 7). The ability of anifrolumab to neutralise the 21-IFNGS in patients was correlated with the levels of IL-10 in a patient at baseline (FIG. 9). Furthermore, there was a higher BICLA response was also observed in IFN-high & IL-10 low patients compared to IFN-high & IL-10 high patients in both the MUSE study (FIG. 7) and TULIP I (FIG. 8).

### 14.3 Effect of Type I IFN Inhibition on IL-10 Levels

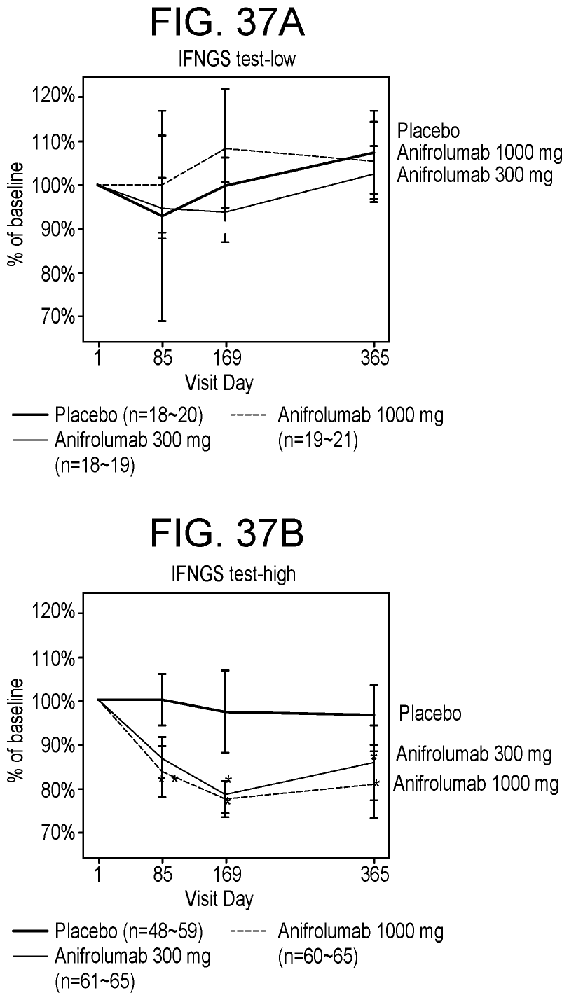

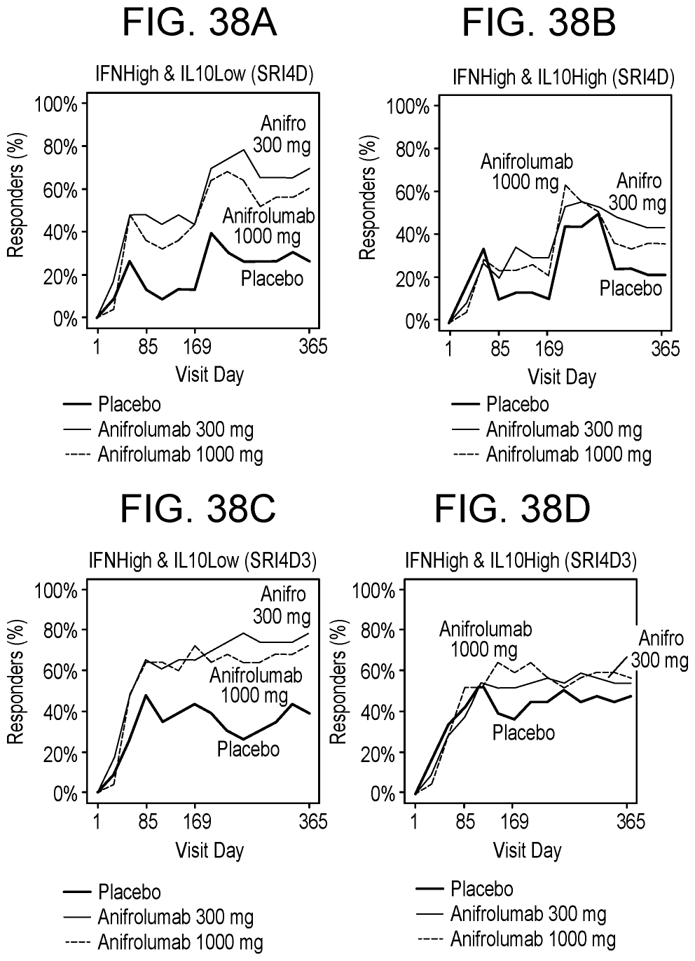

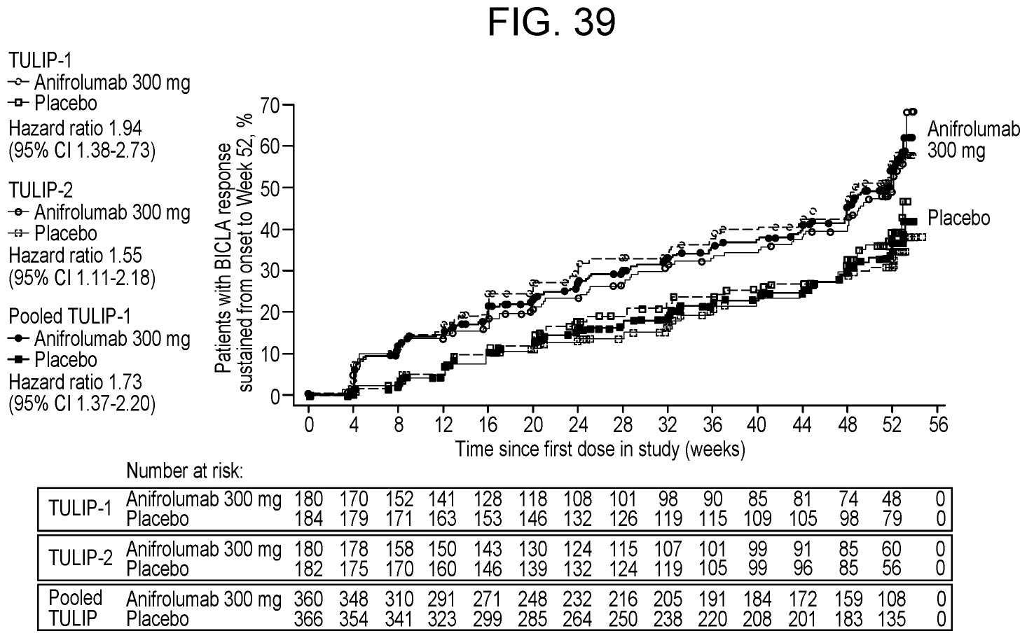

In MUSE, anifrolumab administration significantly suppressed IL-10 plasma levels in SLE patients (FIG. 36). Importantly, IL-10 levels were 2.5 fold higher in IFNGS test-high patients than healthy controls at baseline (FIG. 37A), but anifrolumab-induced IL10 suppression is ˜20% in IFNGS test-high patients, which may not be enough for IL10-high patients (FIG. 38B). There was no evidence of bias over time in the conclusion based on dropouts in either clinical response (FIG. 39).

### 14.4 IL-10 Mechanism of Action in SLE

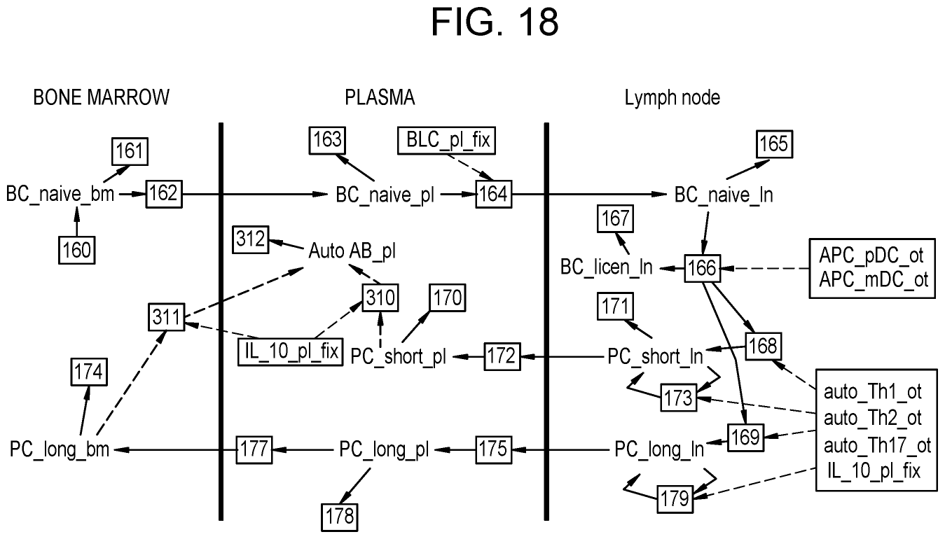

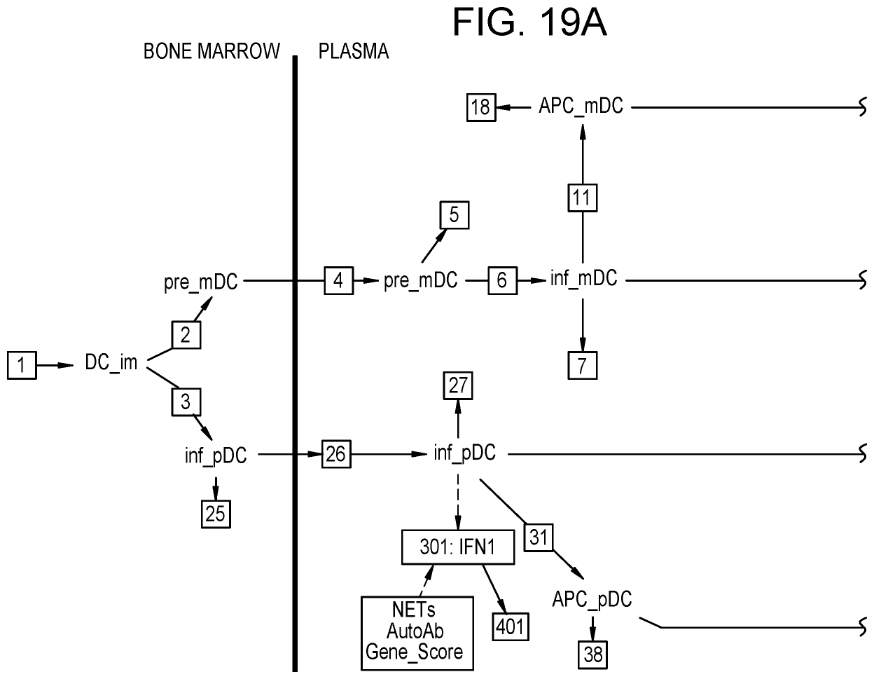

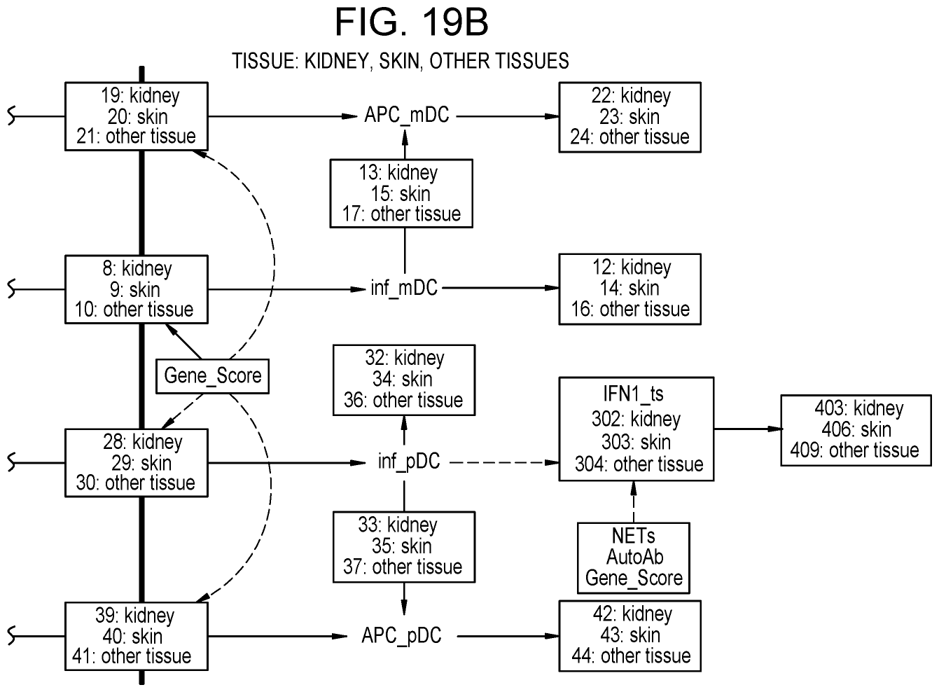

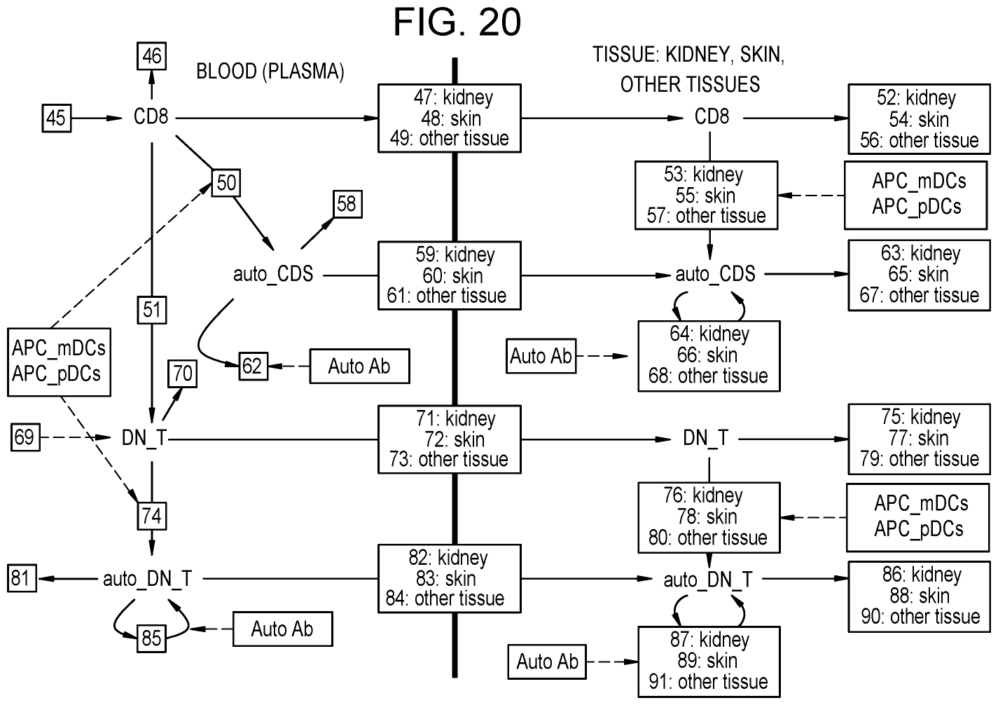

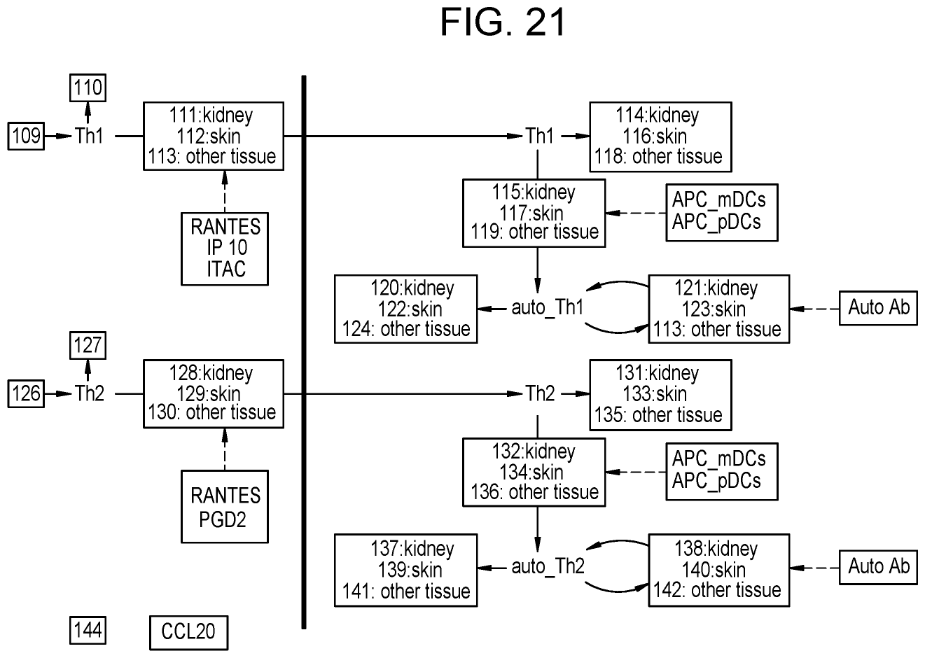

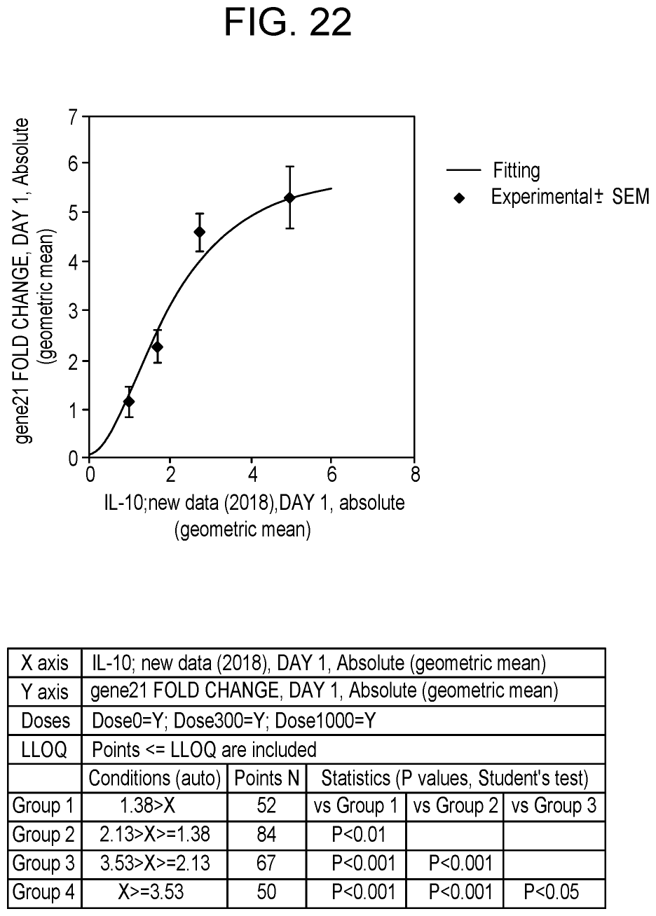

Without being bound by therapy, it is believed that a high IL-10 concentration leads to a hyper-activation of main disease drivers in SLE, for example, type I IFN, auto antibodies and cytotoxic cells, which cannot be sufficiently compensated by anifrolumab (FIG. 18). In particular, the IL-10 may have the following effects:
IL-10 dependent increase of auto-antibodies (AB) leading to an increase of type I IFN production by dendritic cells (FIG. 19).
IL-10 dependent increase of auto-AB leading to a hyper-activation of some important drivers of disease, including type I IFN, interferon stimulated genes, IFNGS, cytotoxic T cells (FIG. 20 and FIG. 21)

### 14.5 Summary

In summary, the present inventors disclose for the first time that high baseline IL-10 is associated with a worse clinical outcome for SLE patients, and surprisingly, IFNGS test-high and IL10-low patients respond better to anifrolumab treatments than other patients. A combination of a type I IFN receptor inhibitor (e.g. anifrolumab) and anti-IL10 antibody would thus plausibly be beneficial to IL10-high patients. IL-10 low patients further represent a sub-population of SLE patients that will respond to treatment with anifrolumab.

### 15 Example 8. Early Sustained Response

### 15.1 Summary

Early and Sustained Responses With Anifrolumab Treatment in Patients With Active Systemic Lupus Erythematosus (SLE) in 2 Phase 3 Trials

### 15.2 Background

In the phase 3 TULIP-2 and TULIP-1 trials in SLE, treatment with the type I interferon receptor antibody anifrolumab resulted in higher percentages of patients with BICLA responses vs placebo at Week 52, with differences of 16.3% (primary endpoint; P=0.001, 95% CI 6.3-26.3) and 16.4% (secondary endpoint; 95% CI 6.7-26.2), respectively.

### 15.3 Objective

To better understand the time course of BICLA responses to anifrolumab, we examined responses over time compared with placebo in TULIP-2 and TULIP-1, including those that were sustained from attainment through Week 52. Major clinical response (MCR) and partial clinical response (PCR) were also assessed as alternative outcome measures. In particular, to compare BICLA responses to anifrolumab vs placebo over time in TULIP-1, TULIP-2, and pooled TULIP data at early time points, the time to onset of response sustained to Week 52 and the major and partial clinical response. The Major clinical response is defined as all BILAG-2004 scores C or better at Week 24, maintained through Week 52, with no new A or B scores between Weeks 24-52. The Partial clinical response is defined as a maximum of 1 BILAG-2004 B score at Week 24, maintained through Week 52, with no new A or >1 new B domain score through Week 52.

### 15.4 Methods

The TULIP-2 and TULIP-1 randomized, double-blind, placebo-controlled trials evaluated the efficacy and safety of anifrolumab (300 mg Q4W) over 52 weeks in patients with moderately to severely active SLE who were receiving standard-of-care treatment. Time to onset of BICLA response that was sustained from attainment through Week 52 was evaluated using a Cox proportional hazards model. MCR was defined as all BILAG-2004 scores C or better at Week 24, maintained with no new A or B scores between Weeks 24-52. PCR was defined as ≤1 BILAG-2004 B score at Week 24 maintained without a new A or >1 new B domain score through Week 52. For TULIP-1, BICLA response rate and time to onset of BICLA response were analyzed using the amended restricted medication rules; MCR and PCR were analyzed using the prespecified analysis plan.

### 15.5 Results

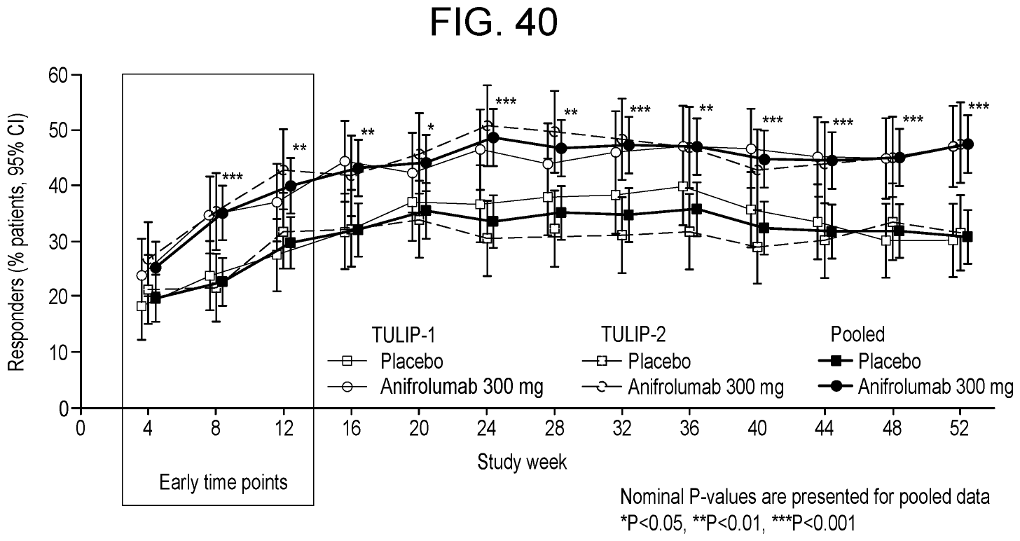

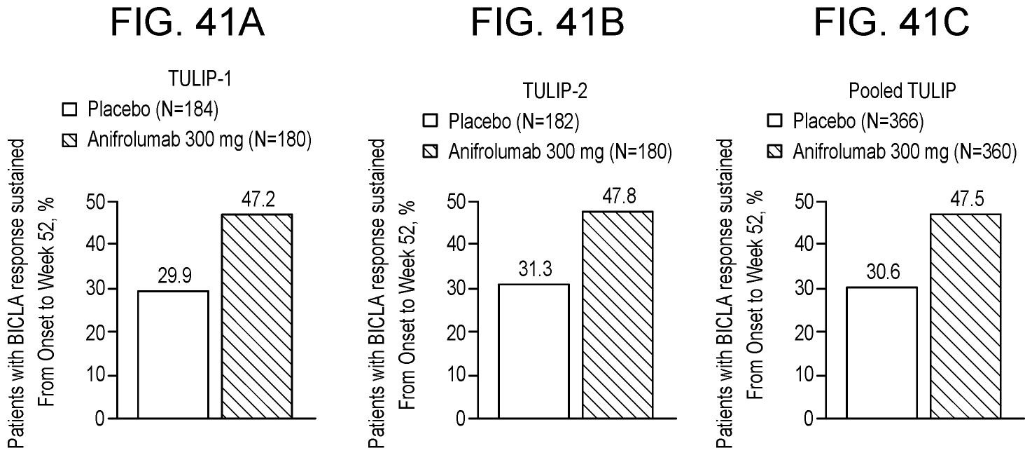

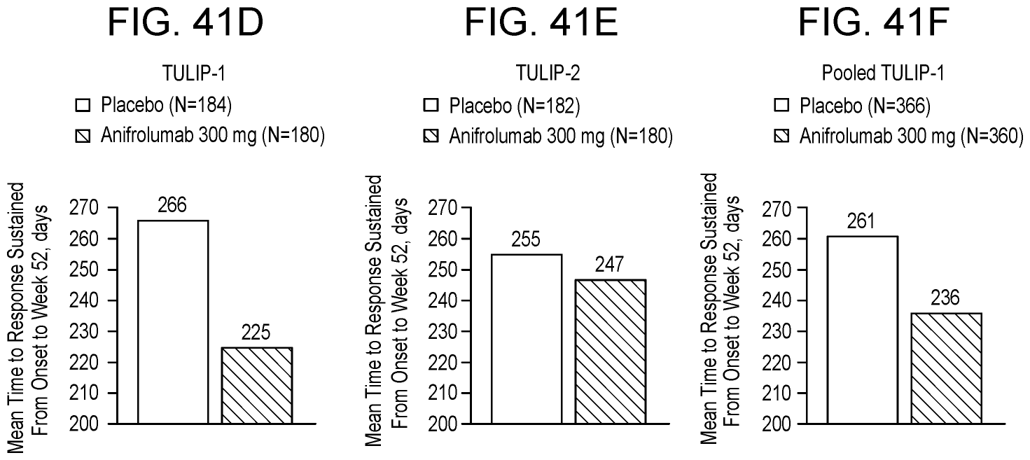

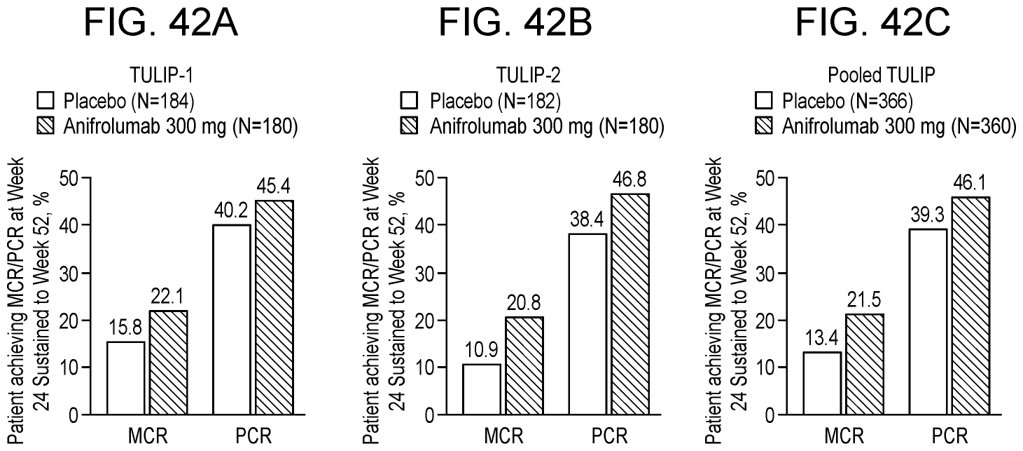

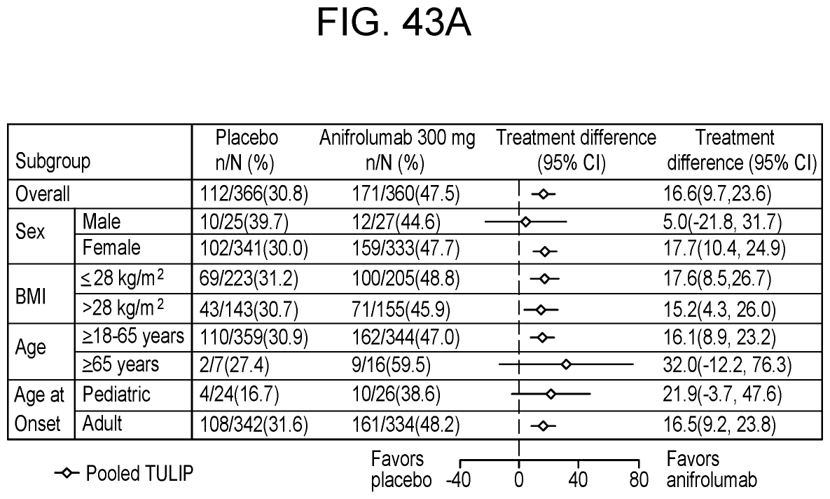

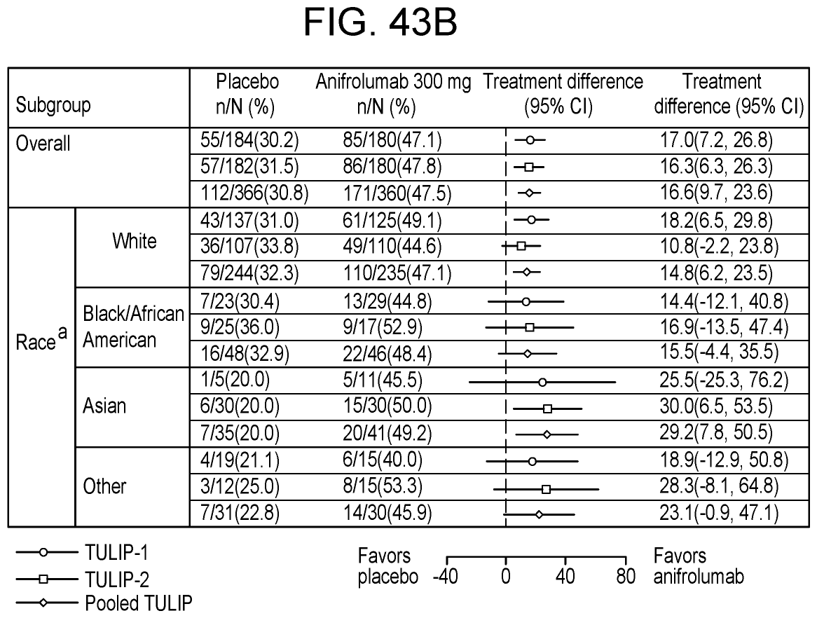

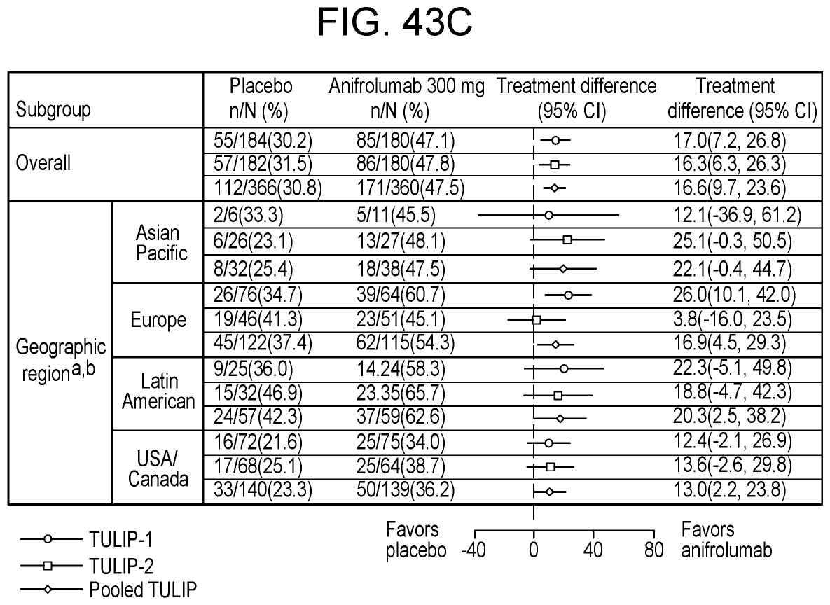

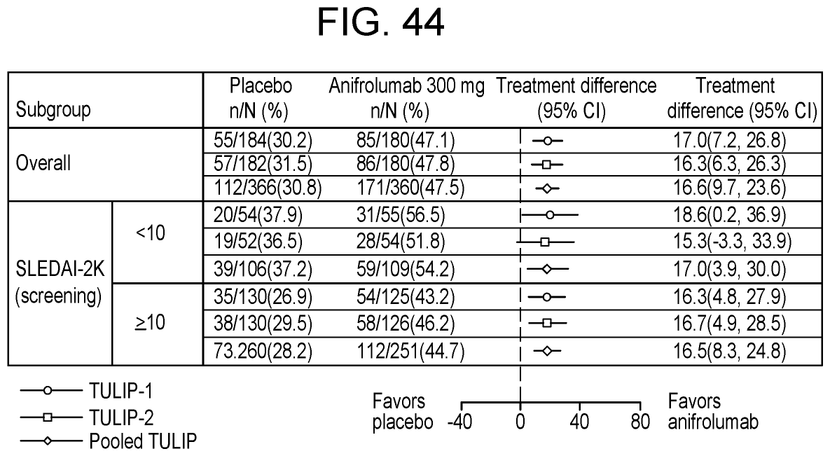

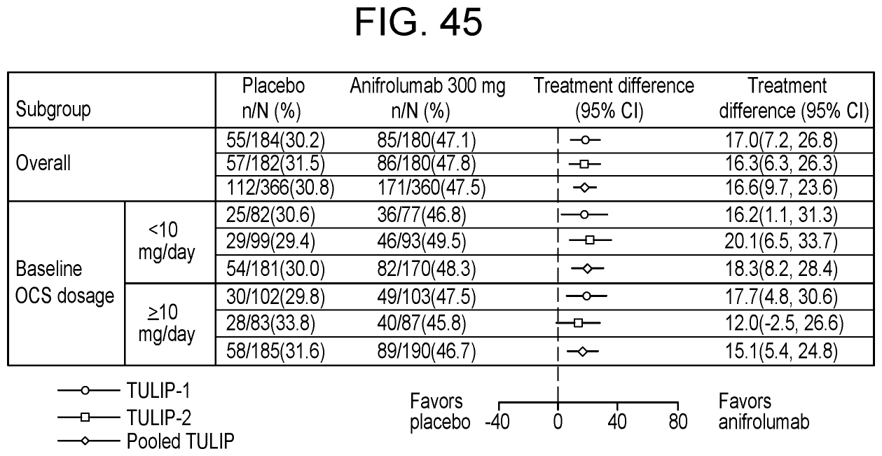

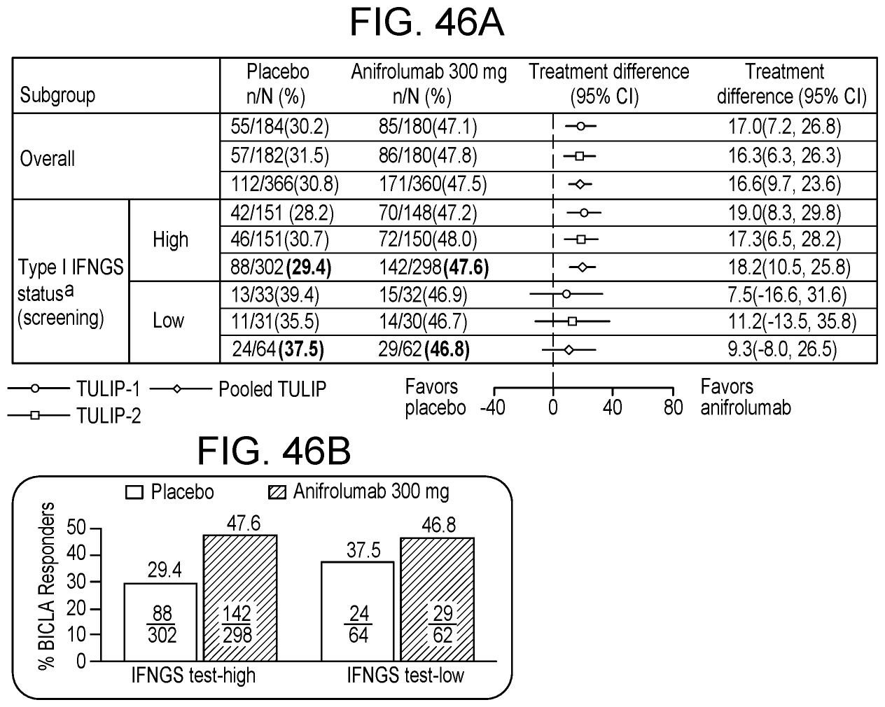

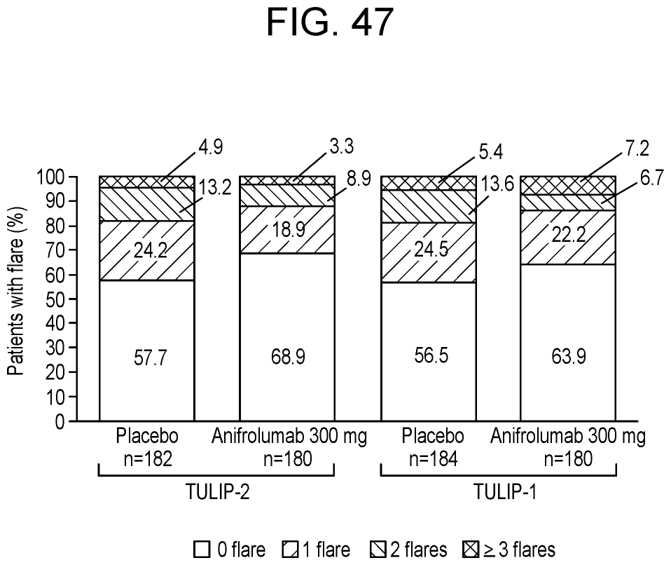

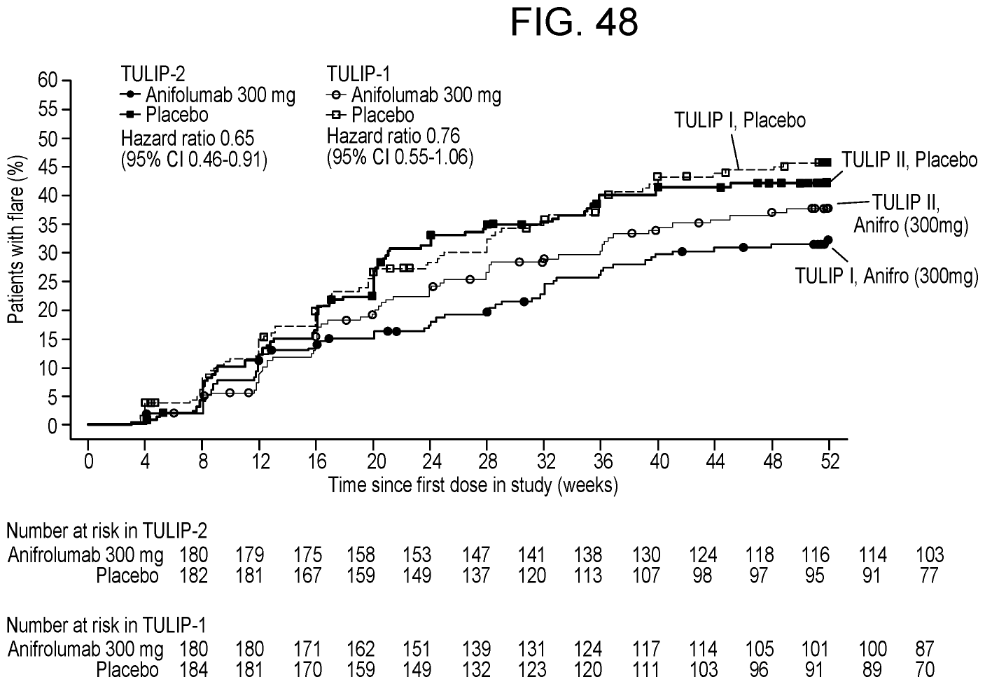

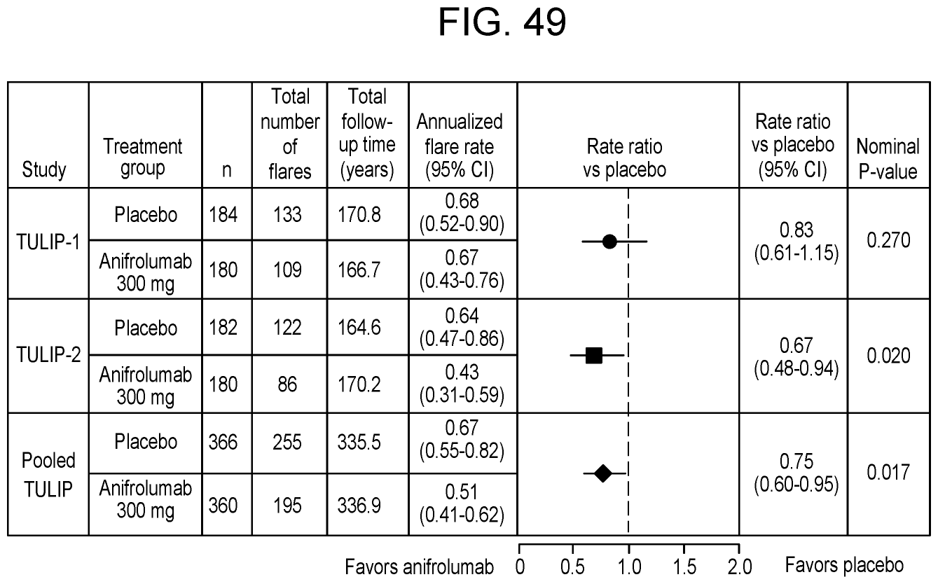

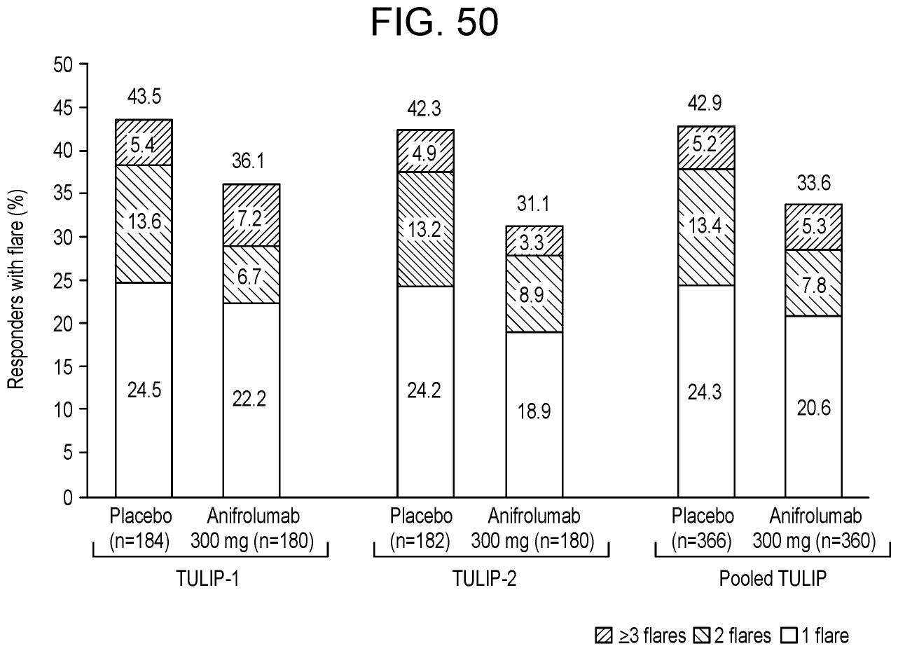

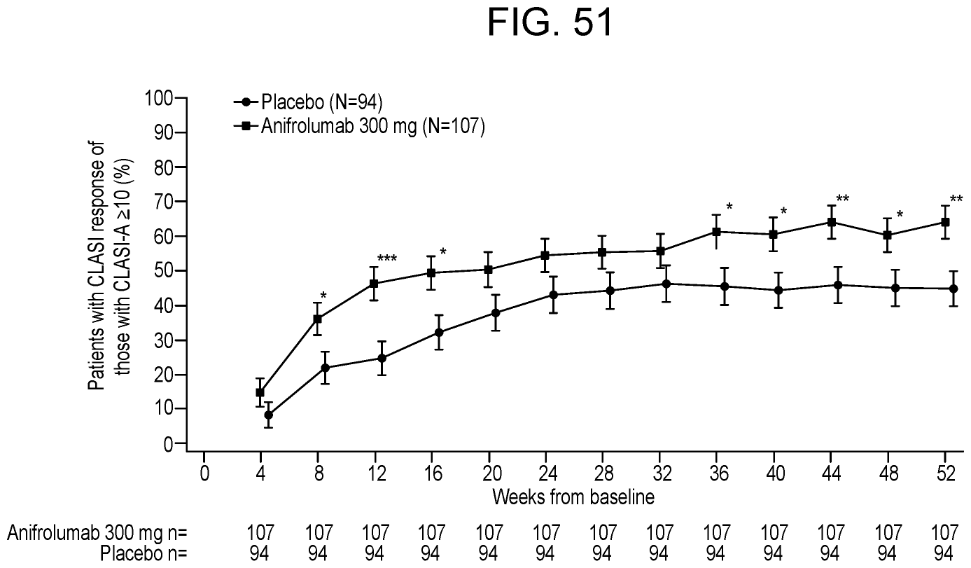

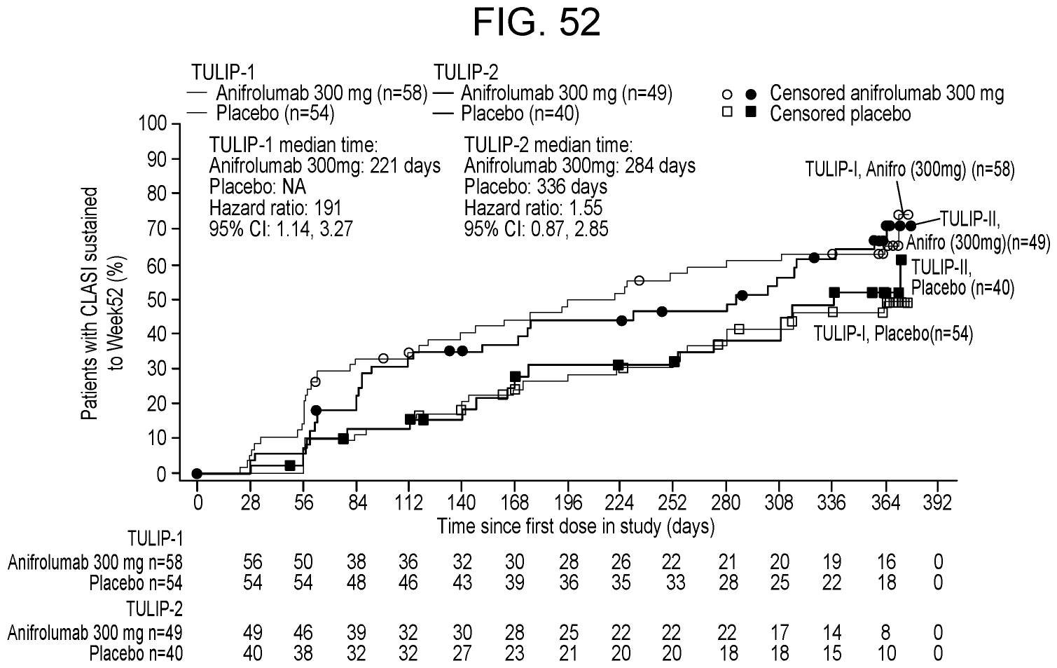

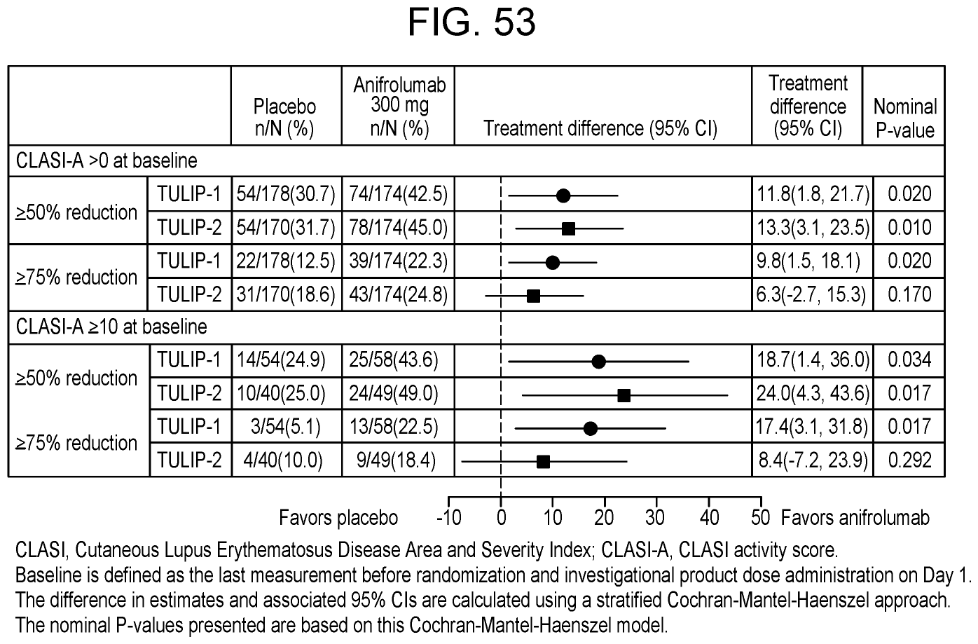

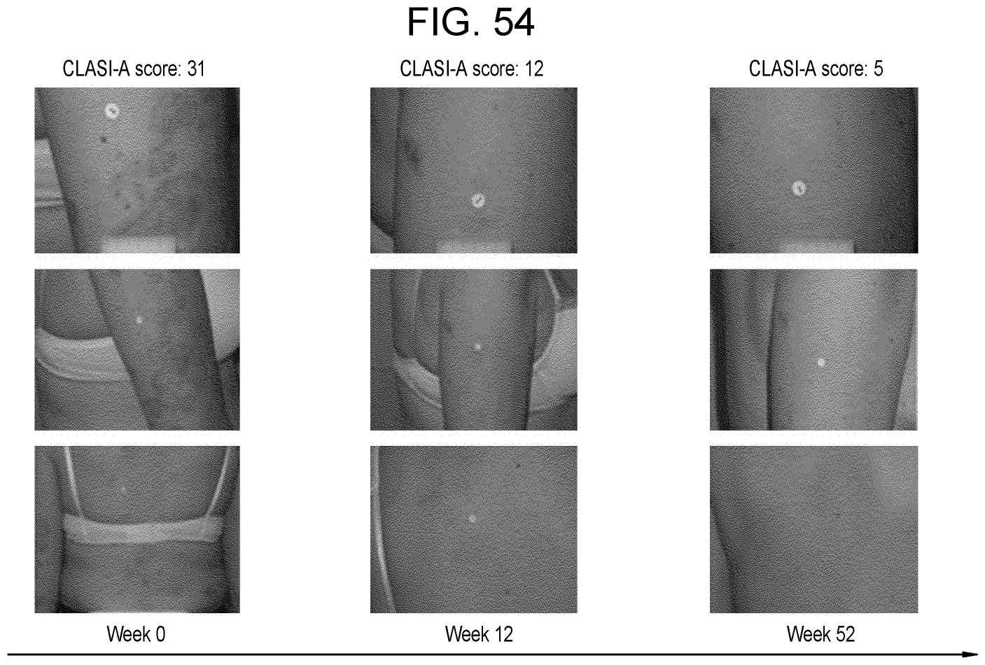

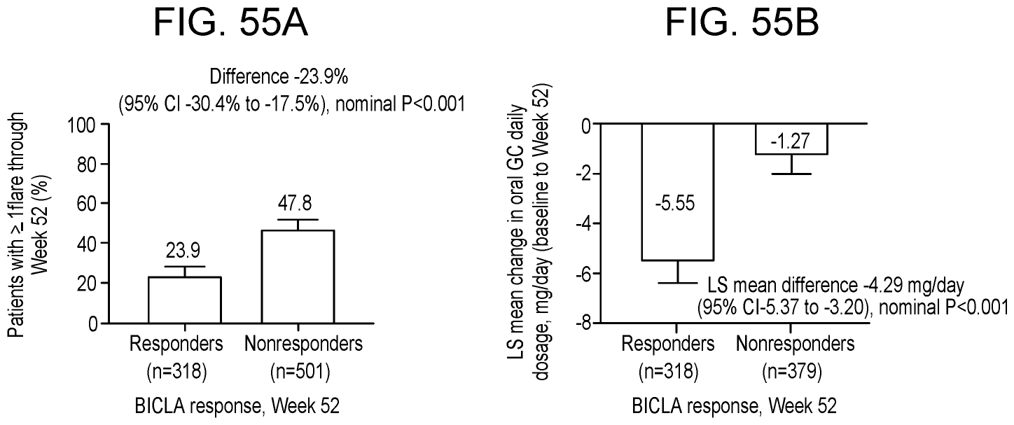

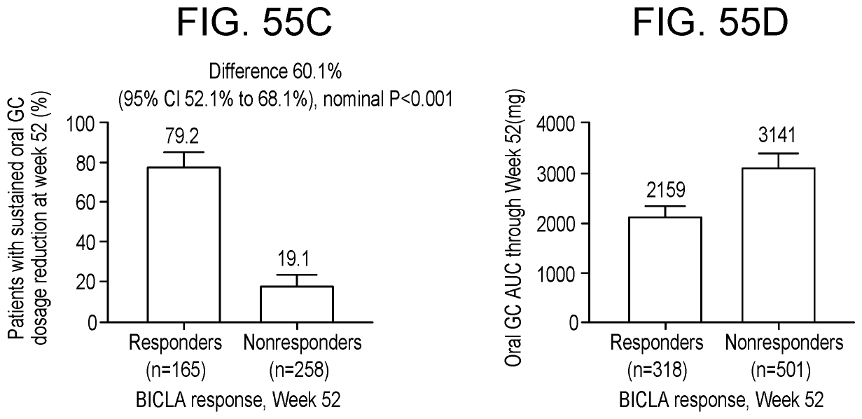

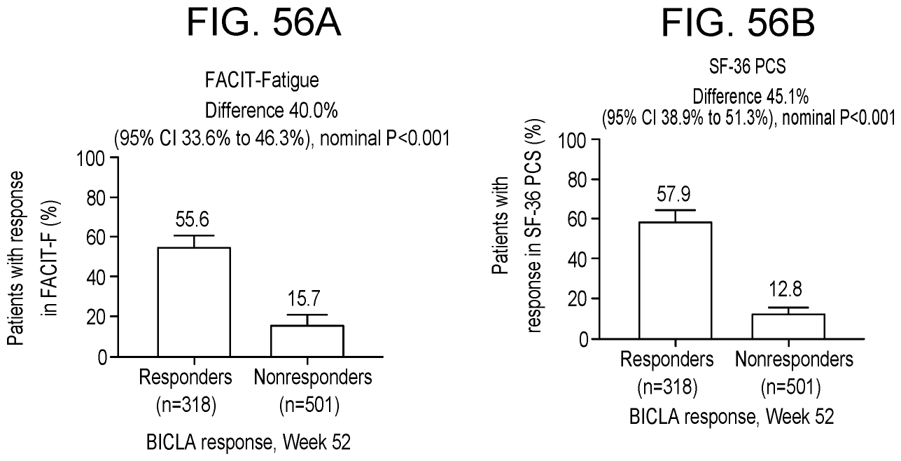

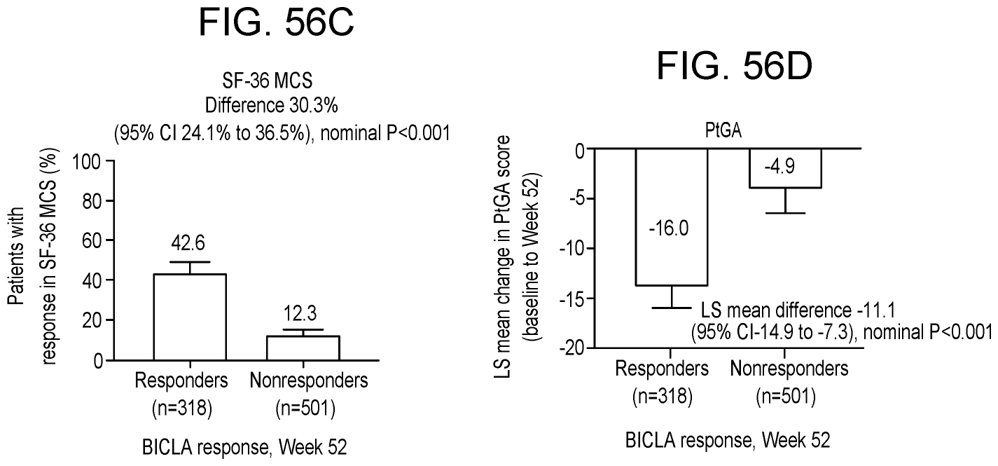

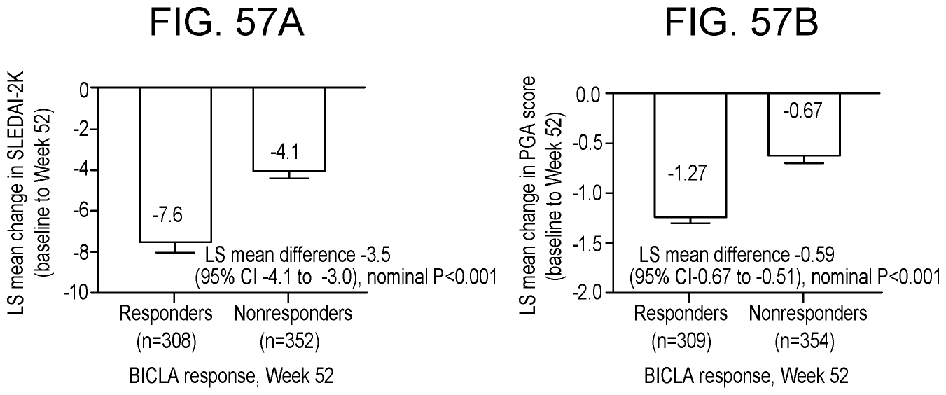

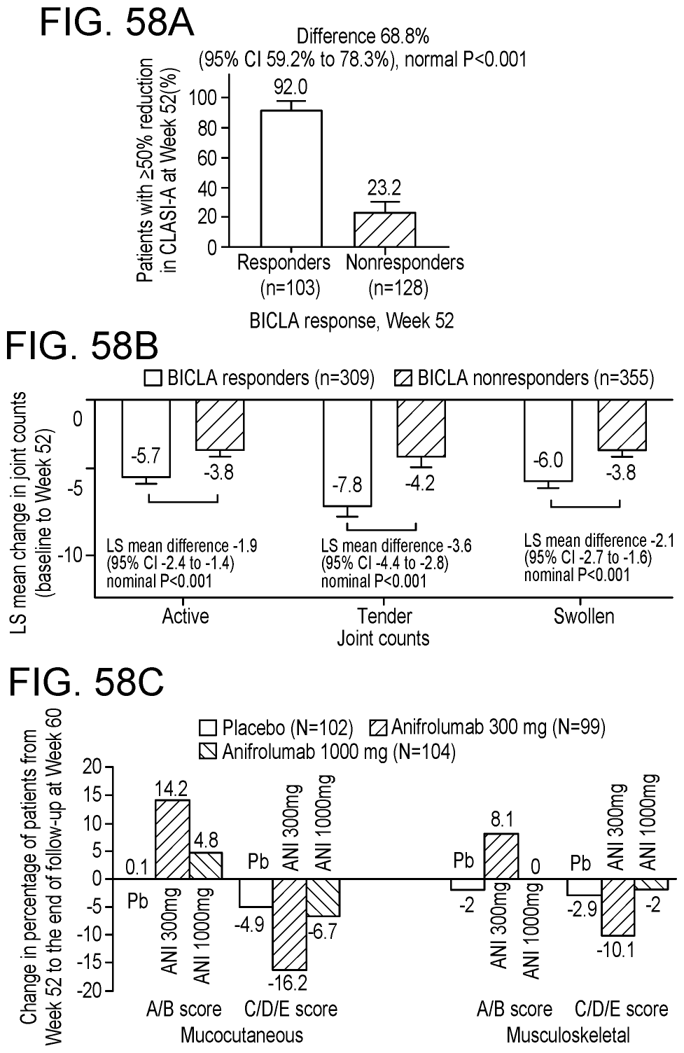

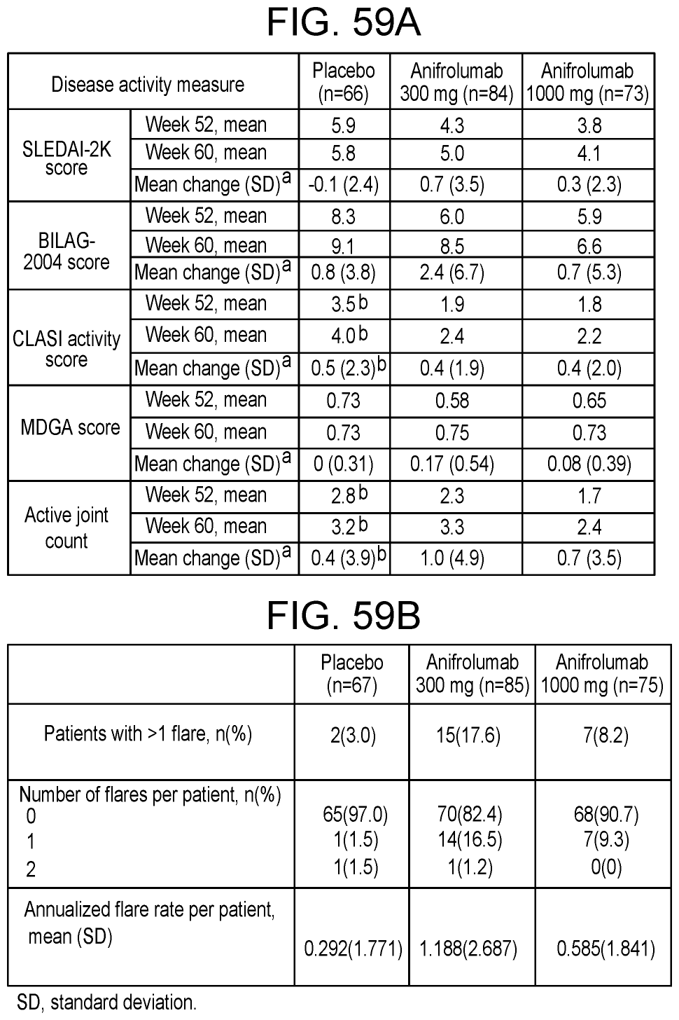

There were more BICLA responders with anifrolumab vs placebo from early time points (FIG. 39). The time to onset of sustained BICLA response favoured anifrolumab (FIG. 39, FIG. 40, FIG. 41A-F). There were numerical differences favouring anifrolumab in the percentage of patients with sustained BICLA responses and the percentage of patients with PCR and MCR (FIG. 42). Overall, 180 patients each in TULIP-2 and TULIP-1 received anifrolumab compared with 182 and 184 patients in the placebo arms, respectively. At the first 3 assessments in TULIP-2 (Weeks 4, 8, and 12), numerically greater percentages of patients treated with anifrolumab (26.8%, 35.3%, and 42.9%, respectively) were classified as having a BICLA response compared with placebo (21.3%, 21.6%, and 31.8%). A similar trend was observed in TULIP-1 with anifrolumab (23.3%, 34.2%, and 36.5%) vs placebo (18.3%, 23.2%, and 27.5%). The time to onset of BICLA response sustained from onset through Week 52 favoured anifrolumab in both TULIP-2 (HR 1.55, 95% CI 1.11-2.18) and TULIP-1 (HR 1.93, 95% CI 1.38-2.73). In TULIP-2, 86 (47.8%) patients treated with anifrolumab had BICLA responses that were sustained from time of onset through Week 52 compared with 57 (31.3%) patients in the placebo group. In TULIP-1, 85 (47.2%) patients in the anifrolumab treatment arm had BICLA responses that were sustained from time of onset through Week 52 compared with 55 (29.9%) patients in the placebo group. In TULIP-2 and TULIP-1, MCR was observed in 20.8% and 22.1% of patients treated with anifrolumab, respectively, compared with 10.9% and 15.8% of those who received placebo. PCR was observed in 46.8% and 45.4% of anifrolumab-treated patients compared with 38.4% and 40.2% in the placebo groups, respectively.

### 15.6 Conclusion

Rapid and durable BICLA responses support the clinical benefit of anifrolumab for patients with moderately to severely active SLE. In 2 Phase 3 studies, a greater proportion of patients achieved BICLA responses sustained from onset through Week 52 with anifrolumab treatment compared with placebo. Anifrolumab resulted in numerically favourable differences in time to onset of BICLA responses maintained through Week 52 across the TULIP studies. MCR and PCR also favoured anifrolumab. These data support the sustainability of clinical benefit derived from anifrolumab treatment of patients with active SLE.

### 16 Example 9: Flare Assessments

### 16.1 Background

Anifrolumab treatment resulted in improved British Isles Lupus Assessment Group (BILAG)-based Composite Lupus Assessment (BICLA) response rates in patients with systemic lupus erythematosus (SLE) in the phase 3 TULIP-2 and TULIP-1 trials. In addition, annualized flare rates were lower among the groups treated with anifrolumab compared with placebo

### 16.2 Objective

TULIP-2 and TULIP-1 data were analyzed to assess the effects of anifrolumab on the number of SLE flares and time to first flare during 52 weeks of treatment.

### 16.3 Methods

The randomized, double-blind, placebo-controlled TULIP-2 and TULIP-1 trials evaluated efficacy and safety of intravenous anifrolumab 300 mg vs placebo every 4 weeks for 48 weeks, with the primary endpoints assessed at Week 52, in patients with moderate to severe SLE despite standard-of-care treatment. Flares were defined as ≥1 new BILAG-2004 A or ≥2 new (worsening) BILAG-2004 B domain scores compared with the prior month's visit. Time to first flare was evaluated using a Cox proportional hazards model. Annualized flare rate was analyzed using a negative binomial regression model.

### 16.4 Results

In TULIP-2 (anifrolumab, n=180; placebo, n=182) and TULIP-1 (anifrolumab, n=180; placebo, n=184), fewer patients experienced ≥1 BILAG-2004 flare in the anifrolumab groups (TULIP-2: 31.1%, n=56; TULIP-1: 36.1%, n=65) compared with the placebo groups (TULIP-2: 42.3%, n=77; TULIP-1: 43. 43.5%, n=80; FIG. 20). Results favouring anifrolumab were observed in time to first flare (TULIP-2: hazard ratio [HR] 0.65, 95% confidence interval [CI] 0.46-0.91 and TULIP-1: HR 0.76, 95% CI 0.55-1.06; FIG. 47) and BILAG-based annualized flare rates (TULIP-2: adjusted rate ratio 0.67, 95% CI 0.48-0.94 and TULIP-1: rate ratio 0.83, 95% CI 0.60-1.14) across both trials. Annualized BILAG flare rates (vs prior visit) were significantly lower in the anifrolumab group compared with the placebo group in TULIP-2 (FIG. 48, FIG. 49). Fewer patients experienced ≥1 BILAG flare vs the prior visit in the anifrolumab group in TULIP-1, TULIP-2 and pooled TULIP (36.1%, 31.1%, and 33.6%, respectively) compared with placebo group (43.5%, 42.3%, and 42.9%, respectively; FIG. 50)

### 16.5 Conclusions

Across 2 phase 3 trials, we observed reductions in the total number of flares and annualized flare rates, as well as prolongation of time to first flare with anifrolumab treatment compared with placebo. These results support the potential of anifrolumab to reduce disease activity and reduce flares, benefiting patients with SLE. The results of the TULIP trials support the capacity of anifrolumab to not only reduce disease activity but also to reduce flares in the presence of OCS taper, an attribute that is vital to the long-term management of patients with SLE.

### 17 Example 10: Early and Sustained Reduction in Severity of Skin Disease as Measured by CLASI

### 17.1 Background

Skin is the second most commonly involved organ in SLE, with up to 85% of patients experiencing skin disease. The Cutaneous Lupus Erythematosus Disease Area and Severity Index (CLASI) is a validated index to measure skin disease severity with activity scores (CLASI-A) ranging from 0 (mild) to 70 (severe). CLASI-A includes measures for erythema, scale/hypertrophy, mucous membrane lesions, recent hair loss, and nonscarring alopecia. In the phase 3 TULIP-1 and-2 trials of patients with SLE, a greater proportion of patients with CLASI-A at baseline ≥10 achieved a ≥50% reduction of CLASI-A at Week 12 with anifrolumab compared with placebo. We further evaluated the effect of anifrolumab on skin-specific SLE disease activity using data pooled from TULIP-1 and -2.

### 17.2 Methods

TULIP-1 and-2 were 52-week randomized, double-blind, placebo-controlled trials that evaluated the efficacy and safety of anifrolumab (300 mg IV every 4 weeks for 48 weeks) in patients with moderately to severely active SLE despite standard-of-care treatment. TULIP-1 and -2 were analyzed separately using restricted medication rules per the TULIP-2 protocol, and data from both trials were pooled. We compared skin responses over time in patients receiving anifrolumab vs placebo. A CLASI-A response was defined as ≥50% reduction of CLASI-A score from baseline for patients with CLASI-A ≥10. Time to CLASI-A response was evaluated using a Cox proportional hazards model.

### 17.3 Results

In total, 360 patients received anifrolumab and 366 received placebo. At baseline, mean (SD) CLASI-A score was 8.1 (7.41); 95.9% (696/726) of patients had baseline CLASI-A >0 and 27.7% (201/726) had baseline CLASI-A ≥10. In the subgroup of patients with baseline CLASI-A ≥10, CLASI-A response (≥50% reduction) was achieved by Week 8 for 36.0% (38/107) of patients receiving anifrolumab vs 21.7% (21/94) receiving placebo (difference 14.3; 95% CI 1.8%, 26.9%). (FIG. 51). Results favouring anifrolumab were observed in time to CLASI-A response sustained to Week 52 in TULIP-1 (hazard ratio [HR] 1.91; 95% CI 1.14, 3.27) and TULIP-2 (HR 1.55; 95% CI 0.87, 2.85) (FIG. 52). Of the subgroup of patients with baseline CLASI-A >0, a greater number of patients achieved a CLASI-A response (≥50% reduction) by Week 12 in the anifrolumab vs placebo groups in both TULIP-1 and-2 (nominal P<0.05) (FIG. 53); a similar effect was observed in the subgroup of patients with baseline CLASI-A ≥10 in both TULIP-1 and -2 (nominal P<0.05). An example from one patient following treatment with anifrolumab (300 mg) is shown in FIG. 54.

### 17.4 Conclusions

Anifrolumab treatment was associated with rapid and durable improvements in skin-specific SLE disease activity, as assessed by CLASI, in a subgroup of patients with mild to severe cutaneous activity at baseline. These findings demonstrate the ability of anifrolumab to reduce skin disease activity in patients with moderately to severely active SLE.

### 18 Example 11: Clinical Relevance of BILCA

### 18.1 Background

The British Isles Lupus Assessment Group-based Composite Lupus Assessment (BICLA) is a validated global measure of treatment response in systemic lupus erythematosus (SLE) clinical trials. To understand the relevance of BICLA to clinical practice, the relationship between BICLA response and routine SLE assessments and patient-reported outcomes (PROs) was investigated.

The BICLA was developed following an expert panel review of disease activity indices used in SLE clinical trials. A BICLA response requires improvement in all domains affected at baseline, assessed by BILAG-2004, no worsening of other BILAG-2004 domains, no worsening versus baseline of both SLEDAI-2K and PGA, no initiation of nonprotocol treatment or use beyond protocol-allowed thresholds, and no discontinuation of investigational product. Thus, in contrast to the SRI, the driver of efficacy in the BICLA is BILAG-2004, whereas worsening is assessed with SLEDAI-2K and PGA in addition to BILAG. BILAG-2004-based BICLA weighs organ systems equally and distinguishes between inactive disease, partial and complete improvement, and deterioration of disease activity, whereas SLEDAI-2K-based SRI assigns weighting to organ systems and requires complete resolution of disease activity in the involved organ system to capture improvement.

Composite SLE assessments are not routinely used in clinical practice. The relevance of treatment response assessed in this way thus may not be appreciated by clinicians. We therefore investigated the relationship between BICLA response and other SLE disease measures that are meaningful in real-world clinical practice, including flares, oral glucocorticoid daily dosage and sustained oral glucocorticoid taper, PROs, medical resource utilization, and clinical and laboratory measures of global and organ-specific disease. These relationships were assessed between BICLA responders and nonresponders using pooled data from the phase 3 TULIP-1 and TULIP-2 trials of anifrolumab, regardless of treatment group assignment.

### 18.2 Methods

### 18.2.1 Patients and Study Design

This was a post hoc analysis of pooled data from the phase 3 randomized, placebo-controlled, double-blind, 52-Week TULIP-1 and TULIP-2 trials. In brief, eligible patients were aged 18 to 70 years, fulfilled the American College of Rheumatology revised classification criteria for SLE (13), and had seropositive moderate to severe SLE despite standard of care treatment. Patients with active severe lupus nephritis or neuropsychiatric SLE were excluded. Patients were randomized to receive intravenous infusions of placebo or anifrolumab every 4 weeks for 48 weeks in addition to standard-of-care treatment (TULIP-1: placebo, anifrolumab 150 mg, or anifrolumab 300 mg [2:1:2]; TULIP-2: placebo or anifrolumab 300 mg [1:1]). Primary endpoints were assessed at Week 52. Other treatments were stable throughout the trial except those resulting from protocol-determined intent-to-taper oral glucocorticoids. For patients receiving oral glucocorticoid ≥10 mg/day at baseline, an attempt to taper oral glucocorticoid to ≥7.5 mg/day was required between Weeks 8 and 40; tapering was also permitted for patients receiving oral glucocorticoid <10 mg/day at baseline. Stable oral glucocorticoid dosage was required between Weeks 40 and 52.

### 18.2.2 Study Endpoints and Assessments

BICLA response was defined as all of the following: reduction of all baseline BILAG-2004 A and B domain scores to B/C/D and C/D, respectively, and no worsening in other BILAG-2004 organ systems as defined by ≥1 new BILAG-2004 A or ≥2 new BILAG-2004 B domain scores; no increase in SLEDAI-2K score (from baseline); no increase in PGA score (≥0.3 points from baseline); no discontinuation of investigational product; and no use of restricted medications beyond protocol-allowed thresholds. Pooled data were analyzed according to the TULIP-2 restricted medication analytical rules to classify responders/nonresponders.

Clinical outcome measures were compared between BICLA responders and nonresponders at Week 52, regardless of treatment group assignment, and results are presented in a hierarchy of clinical relevance, agreed by consensus between the authors. Outcome measures include the percentage of patients with flares (defined as ≥1 new BILAG-2004 A or ≥2 new BILAG-2004 B domain scores compared with the prior visit) through Week 52, annualized flare rates, percentage of patients achieving sustained oral glucocorticoid taper (defined as oral glucocorticoid dosage reduction to ≤7.5 mg/day prednisone or equivalent, achieved by Week 40 and sustained through Week 52, in patients receiving ≥10 mg/day at baseline), and change in daily oral glucocorticoid dosage from baseline to Week 52. Changes in PROs were assessed from baseline to Week 52, including responses in Functional Assessment of Chronic Illness Therapy-Fatigue [FACIT-F] (defined as a >3-point improvement), responses in Short Form 36 Health Survey version 2 [SF-36-v2] [acute] physical component summary [PCS] and mental component summary [MCS] (defined as an improvement of >3.4 in the PCS and >4.6 in the MCS), and changes from baseline in Patient's Global Assessment [PtGA]). Medical resource utilization (health care visits, emergency department [ED] use, and hospital visits) was also assessed. Other indices compared between BICLA responders and nonresponders included changes from baseline to Week 52 in SLEDAI-2K, PGA, joint counts (active, swollen, tender), and the Cutaneous Lupus Erythematosus Disease Area and Severity Index Activity (CLASI-A) responses (defined as ≥50% reduction in CLASI-A score among patients with CLASI-A score ≥10 at baseline). Serologies (anti-double-stranded DNA [anti-dsDNA] antibodies and complement C3 and C4) were evaluated; anti-dsDNA antibody levels were classified as ‘positive’ (>15 U/mL) or ‘negative’ (≤15 U/mL), and complement levels were classified as ‘abnormal’ (C3, <0.9 g/L; C4, <0.1 g/L) or ‘normal’ (C3, ≥0.9 g/L; C4, ≥0.1 g/L). Adverse events (AEs) were also assessed.

### 18.2.3 Statistical Analyses

The similar designs of the TULIP-1 and TULIP-2 studies allowed for the results to be pooled. Sample sizes were selected for TULIP-1 and TULIP-2 to acquire adequate safety database sizes and to assess key secondary endpoints. In TULIP-1 and TULIP-2, 180 subjects/arm yielded >99% and 88% power, respectively, to reject the hypothesis (no difference in the primary endpoint) using a 2-sided alpha of 0.05. Responder versus nonresponder rates were calculated using a stratified Cochran-Mantel-Haenszel approach, which included stratification factors of SLEDAI-2K score at screening (<10 or ≥10), baseline oral glucocorticoid dosage (<10 mg/day or ≥10 mg/day), and type I IFNGS test status at screening (test-low or test-high). Study was also included in the model. For all responder analyses, patients were considered nonresponders if they used restricted medications beyond the protocol-allowed thresholds or discontinued investigational product before the assessment. Comparison of estimated change from baseline to Week 52 between BICLA responders and nonresponders was assessed using a mixed repeated measures model with fixed effects for baseline value, group, visit, study, and the stratification factors used at screening; a group-by-visit interaction term was used and visit was a repeated variable in the model. Missing data were imputed using the last observation carried forward for the first visit with missing data; subsequent visits with missing data were not imputed. For responder analyses, if any component of the variable could not be derived owing to missing data, the patient was classified as a nonresponders for that visit.

### 18.3 Results

### 18.3.1 Trial Populations

Data were pooled for 457 patients in TULIP-1 and 362 patients in TULIP-2 (N=819). Across both trials, 360 patients received anifrolumab 300 mg, 93 patients received anifrolumab 150 mg, and 366 patients received placebo. Regardless of treatment group assignment, there were 318 BICLA responders and 501 BICLA nonresponders at Week 52. Patient demographics and baseline clinical characteristics were generally balanced across BICLA responders and nonresponders (Table 18-1 and Table 18-2). The majority of patients were female (92.5%, responders; 93.0%, nonresponders) and the mean (standard deviation [SDD]) age was 41.5 (11.67) years for responders and 41.7 (12.13) years for nonresponders. Similar proportions of BICLA responders and nonresponders were white (67.0% vs 65.9%), Black/African American (14.2% vs 12.6%), or Asian (9.1% vs 11.0%).

Overall, improved outcomes were observed in BICLA responders versus nonresponders.

TABLE 18-1
Patient demographics and baseline clinical characteristics

| | BICLA | | BICLA | |
|---|---|---|---|---|
| | responders | | nonresponders | |
| | at Week 52 | | at Week 52 | |
| Baseline characteristic | (n = 318) | | (n = 501) | |
| Age, mean (SD), years | 41.5 | (11.67) | 41.7 | (12.13) |
| Female, n (%) | 294 | (92.5) | 466 | (93.0) |
| Time from SLE diagnosis | 85.5 | (0-555) | 84.0 | (4-503) |
| to randomization, median | | | | |
| (range), months | | | | |
| IFNGS test-high at | 261 | (82.1) | 415 | (82.8) |
| screening, n (%) | | | | |
| BILAG-2004 score, | 18.9 | (5.20) | 19.2 | (5.59) |
| mean (SD) | | | | |
| ≥1 A item, n (%) | 171 | (53.8) | 222 | (44.3) |
| No A and ≥2 B items, | 126 | (39.6) | 254 | (50.7) |
| n (%) | | | | |
| No A and <2 B items, | 21 | (6.6) | 25 | (5.0) |
| n (%) | | | | |
| SLEDAI-2K score, | 10.8 | (3.19) | 11.7 | (4.01) |
| mean (SD) | | | | |
| <10, n (%) | 104 | (32.7) | 127 | (25.3) |
| ≥10, n (%) | 214 | (67.3) | 374 | (74.7) |
| PGA score, mean (SD) | 1.76 | (0.425) | 1.81 | (0.396) |
| Oral glucocorticoid | 263 | (82.7) | 410 | (81.8) |
| use,^{a} n (%) | | | | |
| <10 mg/day | 98 | (30.8) | 152 | (30.3) |
| ≥10 mg/day | 165 | (51.9) | 258 | (51.5) |
| Antimalarials | 225 | (70.8) | 361 | (72.1) |
| Immunosuppressants | 158 | (49.7) | 230 | (45.9) |
| CLASI activity score, | 8.5 | (7.56) | 7.8 | (7.18) |
| mean (SD) | | | | |
| <10, n (%) | 215 | (67.6) | 373 | (74.5) |
| ≥10, n (%) | 103 | (32.4) | 128 | (25.5) |
| 0, n (%) | 13 | (4.1) | 20 | (4.0) |
| >0, n (%) | 305 | (95.9) | 481 | (96.0) |
| SDI global score, mean (SD) | 0.6 | (1.08) | 0.5 | (0.89) |
| Swollen joint count, | 6.5 | (5.27) | 7.4 | (6.08) |
| mean (SD) | | | | |
| Tender joint count, | 9.8 | (6.94) | 11.1 | (7.85) |
| mean (SD) | | | | |
| Active joint count,^{b} | 6.1 | (5.22) | 6.9 | (5.97) |
| mean (SD) | | | | |
| Anti-dsDNA positive,^{c} | 142 | (44.7) | 224 | (44.7) |
| n (%) | | | | |
| Median (min, max),^{d} | 48.2 | (15, 3790) | 57.0 | (15, 4404) |
| U/mL | | | | |
| Mean (SD),^{c} U/mL | 142.5 | (401.84) | 220.4 | (526.38) |
| Abnormal C3,^{e} n (%) | 123 | (38.7) | 178 | (35.5) |
| Median (min, max),^{c} g/L | 0.729 | (0.36, 0.90) | 0.715 | (0.18, 0.90) |
| Mean (SD),^{c} g/L | 0.711 | (0.1279) | 0.685 | (0.1603) |
| Abnormal C4,^{f} n (%) | 72 | (22.6) | 118 | (23.6) |
| Median (min, max),^{c} g/L | 0.080 | (0.05, 0.10) | 0.072 | (0.05, 0.10) |
| Mean (SD),^{c} g/L | 0.075 | (0.0168) | 0.071 | (0.0145) |

BICLA, BILAG-based Composite Lupus Assessment; BILAG-2004, British Isles Lupus Assessment Group-2004; CLASI, Cutaneous Lupus Erythematosus Disease Area and Severity Index; dsDNA, double-stranded DNA; IFNGS, interferon gene signature; max, maximum; min, minimum; PGA, Physician's Global Assessment; SD, standard deviation; SDI, Systemic Lupus International Collaborating Clinics/American College of Rheumatology Damage Index; SLE, systemic lupus erythematosus; SLEDAI-2K, SLE Disease Activity Index 2000.
^{a}Oral glucocorticoid includes prednisone or equivalent;
^{b}An active joint is defined as a joint with swelling and tenderness;
^{c}Anti-dsDNA antibody ‘positive’ defined as a result of >15 U/mL.
^{d}Only patients with anti-dsDNA antibodies and abnormal complement levels at baseline are included in the summary statistics for the respective variables.
^{e}Complement C3 ‘abnormal’ levels defined as a result of <0.9 g/L.
^{f}Complement C4 ‘abnormal’ levels defined as a result of <0.1 g/L.

TABLE 18-2
Patient demographics and baseline SLE medications
for BICLA responders and nonresponders

| | | |
|---|---|---|
| | BICLA | BICLA |
| | responders | nonresponders |
| Baseline demographics | at Week 52 | at Week 52 |
| and SLE medication | (n = 318) | (n = 501) |

Race,^{a} n (%)

| | | | | |
|---|---|---|---|---|
| White | 213 | (67.0) | 330 | (65.9) |
| Black/African American | 45 | (14.2) | 63 | (12.6) |
| Asian | 29 | (9.1) | 55 | (11.0) |
| Other | 23 | (7.2) | 45 | (9.0) |
| Hispanic or Latino ethnic | 80 | (25.2) | 115 | (23.0) |
| group, n (%) | | | | |

Geographic region, n (%)

| | | | | |
|---|---|---|---|---|
| United States/Canada | 92 | (28.9) | 226 | (45.1) |
| Europe | 126 | (39.6) | 144 | (28.7) |
| Latin America | 66 | (20.8) | 63 | (12.6) |
| Asia Pacific | 28 | (8.8) | 49 | (9.8) |
| Rest of world | 6 | (1.9) | 19 | (3.8) |

Baseline treatment for SLE, n (%)

| | | | | |
|---|---|---|---|---|
| Oral glucocorticoid^{b} | 263 | (82.7) | 410 | (81.8) |
| Antimalarial | 225 | (70.8) | 361 | (72.1) |
| Immunosuppressant^{c} | 158 | (49.7) | 230 | (45.9) |

BICLA, British Isles Lupus Assessment Group-based Composite Lupus Assessment;; SD, standard deviation; SLE, systemic lupus erythematosus.
^{a}Race data were missing from 16 patients (8 each in the responder and nonresponder groups);
^{b}Oral glucocorticoid included prednisone or equivalent;
^{c}Immunosuppressant agents included azathioprine, methotrexate, mycophenolate mofetil, mycophenolic acid, and mizoribine.

### 18.3.2 Flares

More BICLA responders than nonresponders were flare free over the 52-week treatment period (76.1% vs 52.2%), meaning that fewer BICLA responders than nonresponders experienced ≥1 flare over the 52-week period (23.9% vs 47.8%; difference −23.9%; 95% confidence interval [CI] −30.4 to −17.5; nominal P<0.001) (FIG. 56A). Fewer patients experienced 1, 2, or ≥3 flares and the annualized flare rate was lower for BICLA responders versus nonresponders (rate ratio [RR] 0.36, 95% CI 0.29 to 0.47; nominal P<0.001) (Table 18-3).

TABLE 18-3
SLE flares in BICLA responders and nonresponders

| | |
|---|---|
| BICLA | BICLA |
| responders at | nonresponders |
| Week 52 | at Week 52 |
| (n = 318) | (n = 501) |

Flares per patient through Week 52

| Patients with 0 flares, | 242 | (76.1) | 263 | (52.2) |
|---|---|---|---|---|
| n (%)^{a} | | | | |
| Patients with ≥1 flare, | 76 | (23.9) | 238 | (47.8) |
| n (%) | | | | |
| Comparison between | −23.9 (−30.4 | | | |
| groups, difference | to −17.5), <0.001 | | | |
| (95% CI), nominal P | | | | |
| valuea | | | | |
| 1 | 52 | (16.4) | 132 | (26.3) |
| 2 | 18 | (5.7) | 69 | (13.8) |
| ≥3 | 6 | (1.9) | 37 | (7.4) |

Flare rate through Week 52^{b}

| Total number of flares | 107 | | 401 | |
|---|---|---|---|---|
| Total follow-up time, | 318.8 | | 440.6 | |
| years | | | | |
| Annualized rate | 0.30 | (0.24 to 0.38) | 0.83 | (0.70 to 0.98) |
| (95% CI) | | | | |

| | |
|---|---|
| Comparison between | 0.36 (0.29 |
| groups, rate ratio | to 0.47), <0.0001 |
| (95% CI), nominal P | |
| value | |

BICLA, BILAG-based Composite Lupus Assessment; BILAG-2004, British Isles Lupus Assessment Group-2004; CI, confidence interval; CMH, Cochran-Mantel-Haenszel; PGA, Physician's Global Assessment; SD, standard deviation; SLE, systemic lupus erythematosus; SLEDAI-2K, SLE Disease Activity Index 2000. BILAG-2004 flares were defined by ≥1 new BILAG-2004 A or ≥2 new BILAG-2004 domain scores compared with the prior visit.
^{a}Percentages, difference, Cl, and nominal P values are weighted and calculated using a stratified CMH approach.
^{b}Flare rates calculated using a negative binomial regression model which included covariates of group and stratification factors. The logarithm to the (base e) of the follow-up time is used as an offset variable in the model to adjust for different exposure times.

### 18.3.3 Oral Glucocorticoid Use and Steroid Sparing

Similar percentages of BICLA responders and nonresponders were receiving oral glucocorticoid at any dosage, and at ≥10 mg/day, at baseline. BICLA responders versus nonresponders had greater reductions in daily oral glucocorticoid dosage from baseline to Week 52 (least squares [LS] mean difference −4.29 mg/day, 95% CI −5.37 to −3.20, nominal P<0.001) (FIG. 55B). The secondary endpoint of sustained oral glucocorticoid dosage reduction to ≤7.5 mg/day among patients who were receiving oral glucocorticoid ≥10 mg/day at baseline was achieved by more BICLA responders than nonresponders (79.2% vs 19.1%; difference 60.1%, 95% CI 52.1% to 68.1%, nominal P<0.001) (FIG. 55C). Mean (SD) cumulative oral glucocorticoid dose through Week 52 was 31.3% lower in BICLA responders versus nonresponders (2159.20 [1661.39] mg vs 3140.81 [3081.19] mg) (FIG. 55D).

### 18.3.4 PROs

FACIT-F, SF-36 MCS, and SF-36 PCS scores were similar for BICLA responders and nonresponders at baseline (Table 18-4). Improvement in FACIT-F was reported in more BICLA responders than nonresponders (55.6% vs 15.7%; difference 40.0%, 95% CI 33.6% to 46.3%, nominal P<0.001) (FIG. 56A). Similarly, more BICLA responders than nonresponders had improvement above the predefined threshold in SF-36 PCS (57.9% vs 12.8%; difference 45.1%, 95% CI 38.9% to 51.3%, nominal P<0.001) and SF-36 MCS (42.6% vs 12.3%; difference 30.3%, 95% CI 24.1% to 36.5%, nominal P<0.001) (FIG. 56A-C).

TABLE 18-4
PRO scores at baseline in BICLA responders and nonresponders

| | | |
|---|---|---|
| | BICLA | BICLA |
| | responders | nonresponders |
| PRO | (n = 318) | (n = 501) |

FACIT-F

| | | |
|---|---|---|
| n | 311 | 467 |

| | | | | |
|---|---|---|---|---|
| Mean (SD) | 26.3 | (12.63) | 25.3 | (11.99) |

SF-36 PCS

| | | |
|---|---|---|
| n | 312 | 469 |

| | | | | |
|---|---|---|---|---|
| Mean (SD) | 38.3 | (9.20) | 37.0 | (9.25) |

SF-36 MCS

| | | |
|---|---|---|
| n | 312 | 469 |

| | | | | |
|---|---|---|---|---|
| Mean (SD) | 43.8 | (11.35) | 44.0 | (11.24) |

PtGA

| | | |
|---|---|---|
| n | 311 | 467 |

| | | | | |
|---|---|---|---|---|
| Mean (SD) | 54.7 | (23.36) | 55.8 | (21.44) |

BICLA, British Isles Lupus Assessment Group-based Composite Lupus Assessment; FACIT-F Functional Assessment of Chronic Illness Therapy-Fatigue; MCS, mental component score; PCS, physical component score; PRO, patient-reported outcome; PtGA, Patient's Global Assessment; SD, standard deviation; SF-36-v2, Short Form 36 Health Survey version 2 (acute recall).

### 18.3.5 PtGA

PtGA scores were similar for BICLA responders and nonresponders at baseline. Greater improvements in PtGA scores from baseline to Week 52 were reported for BICLA responders than nonresponders (LS mean difference−11.1, 95% CI −14.9 to −7.3, nominal P<0.001) (FIG. 56D).

### 18.3.6 Medical Resource Utilization

During the 52-week trials, fewer BICLA responders than nonresponders had health care visits (62.5% vs 70.7%; difference −8.3%, 95% CI −14.9% to −1.6%, nominal P=0.015) (Table 18-5). Fewer BICLA responders required emergency department (ED) visits compared with nonresponders (11.9% vs 21.8%; difference −9.9%, 95% CI −15.2% to −4.5%, nominal P=0.001), and fewer ED visits were related to increased SLE activity (2.6% vs 24.0%; difference −21.4%, 95% CI −35.3% to −7.5%, nominal P=0.003). Similarly, fewer BICLA responders than nonresponders had hospital visits (4.5% vs 14.4%; difference −10.0%, 95% CI −14.3% to −5.7%, nominal P<0.001), and no hospital visits were related to increased SLE activity among BICLA responders, compared with 38.5% among BICLA nonresponders (difference −38.5%, 95% CI −58.8% to −18.2%, nominal P<0.001).

TABLE 18-5
Medical resource utilization for BICLA
responders and nonresponders

| | | |
|---|---|---|
| | BICLA | BICLA |
| | responders at | nonresponders |
| | Week 52 | at Week 52 |
| Medical resource utilization | (n = 318) | (n = 501) |

Health care visits (specialist and primary care)

| | | | | |
|---|---|---|---|---|
| Patients with ≥1 health care | 198 | (62.5) | 348 | (70.7) |
| visit,^{a} n (%) | | | | |

| | |
|---|---|
| Comparison between groups, | −8.3 (−14.9 |
| difference (95% CI), nominal | to −1.6), 0.015 |
| P value | |

Emergency department visits

| Patients with ≥1 ED visit,^{a} n (%) | 38 | (11.9) | 107 | (21.8) |
|---|---|---|---|---|
| Comparison between groups, | −9.9 (−15.2 | | | |
| difference (95% CI), nominal | to −4.5), <0.001 | | | |
| P value | | | | |
| Visit related to increase in | 1 | (2.6) | 25 | (24.0) |
| SLE activity,^{a} n (%) | | | | |
| Comparison between groups, | −21.4 (−35.3 | | | |
| difference (95% CI), nominal | to −7.5), 0.003 | | | |
| P value | | | | |
| Number of ED visits per | 1.4 | (0.86) | 1.7 | (1.48) |
| patient,^{b} mean (SD) | | | | |

Hospitalizations

| Patients with ≥1 hospital | 14 | (4.5) | 71 | (14.4) |
|---|---|---|---|---|
| visits,^{a} n (%) | | | | |
| Comparison between groups, | −10.0 (−14.3 | | | |
| difference (95% CI), | to −5.7), <0.001 | | | |
| nominal P value | | | | |
| Visit related to increase in | 0 | | 25 | (38.5) |
| SLE activity,^{a} n (%) | | | | |
| Comparison between groups, | −38.5 (−58.8 | | | |
| difference (95% CI), | to −18.2), <0.001 | | | |
| nominal P value | | | | |
| Number of hospital visits per | 1.6 | (2.13) | 1.4 | (0.72) |
| patient,^{b} mean (SD) | | | | |
| Length of hospital stay,^{b} | 5.7 | (2.64) | 7.4 | (8.02) |
| mean (SD), days | | | | |

| Total days in ICU,^{b} n | 0 | 5 | |
|---|---|---|---|
| Number of days in ICU, mean (SD) | — | 1.8 | (0.45) |

BICLA, British Isles Lupus Assessment Group-based Composite Lupus Assessment; CI, confidence interval; ED, emergency department; ICU, intensive care unit; SD, standard deviation; SLE, systemic lupus erythematosus.
^{a}Percentages, differences, Cls, and nominal P values were calculated using a stratified CMH approach.
^{b}Data on hospital visits and emergency department visits were missing from 8 patients in the BICLA nonresponders group.

### 18.3.7 SLEDAI-2K and PGA

Mean (SD) SLEDAI-2K and PGA scores were similar between responders and nonresponders at baseline (Table 1). From baseline to Week 52, greater improvements were observed for BICLA responders versus nonresponders in total SLEDAI-2K (LS mean difference −3.5, 95% CI −4.1 to −3.0, nominal P<0.001) (FIG. 57A) and PGA scores (LS mean difference −0.59, 95% CI −0.67 to −0.51, nominal P<0.001) (FIG. 57B).

### 18.3.8 CLASI Activity

Overall, 32.4% of BICLA responders and 25.5% of nonresponders had a baseline CLASI-A ≥10 (Table 1). Among these patients, more BICLA responders achieved a ≥50% reduction in CLASI-A at Week 52 versus nonresponders (92.0% vs 23.2%; difference 68.8%, 95% CI 59.2% to 78.3%, nominal P<0.001) (FIG. 31A).

### 18.3.9 Joint Counts

Mean (SD) active joint counts (defined as joints with swelling and tenderness) were 6.1 (5.22) and 6.9 (5.97) in BICLA responders and nonresponders, respectively, at baseline. Mean (SD) swollen joint counts were 6.5 (5.27) and 7.4 (6.08), respectively, and tender joint counts were 9.8 (6.94) and 11.1 (7.85), respectively. From baseline to Week 52, joint counts improved more for BICLA responders versus nonresponders for active (LS mean difference −1.9, 95% CI −2.4 to −1.4, nominal P<0.001), tender (LS mean difference −3.6, 95% CI −4.4 to −2.8, nominal P<0.001), and swollen (LS mean difference −2.1, 95% CI −2.7 to −1.6, nominal P<0.001) joints (FIG. 58B).

### 18.3.10 Serologies

Equal percentages of patients were anti-dsDNA antibody positive at baseline between BICLA responders and nonresponders. Improvement from positive to negative anti-dsDNA antibody status \\was observed in similar percentages of BICLA responders and nonresponders (5.0% vs 4.4%) (Table 18-6).

TABLE 18-6
Serology changes from baseline to Week 52
for BICLA responders and nonresponders

| | | |
|---|---|---|
| | BICLA | BICLA |
| | responders | nonresponders |
| | at Week 52 | at Week 52 |
| Change from baseline to Week 52 | (n = 318) | (n = 501) |

Anti-dsDNA^{a}

| n | 132 | | 147 | |
|---|---|---|---|---|
| Shift positive to negative, n (%) | 16 | (5.0) | 22 | (4.4) |
| Shift negative to positive, n (%) | 8 | (2.5) | 16 | (3.2) |
| Remained negative, n (%) | 114 | (35.8) | 142 | (28.3) |
| Remained positive, n (%) | 116 | (36.5) | 125 | (25.0) |

Complement C3^{b}

| n | 121 | | 127 | |
|---|---|---|---|---|
| Shift abnormal to normal, n (%) | 33 | (10.4) | 35 | (7.0) |
| Shift normal to abnormal, n (%) | 14 | (4.4) | 30 | (6.0) |
| Remained normal, n (%) | 167 | (52.5) | 193 | (38.5) |
| Remained abnormal, n (%) | 88 | (27.7) | 92 | (18.4) |
| LS mean change, % (95% CI) | 15.7 | (9.489 to 21.926) | 12.9 | (6.852 to 18.929) |

| | |
|---|---|
| LS mean difference, % (95% CI), | 2.8 (−4.19 |
| nominal P value | to 9.82), 0.429 |

Complement C4^{c}

| n | 70 | | 79 | |
|---|---|---|---|---|
| Shift abnormal to normal, n (%) | 24 | (7.5) | 24 | (4.8) |
| Shift normal to abnormal, n (%) | 9 | (2.8) | 20 | (4.0) |
| Remained normal, n (%) | 222 | (69.8) | 251 | (50.1) |
| Remained abnormal, n (%) | 46 | (14.5) | 55 | (11.0) |
| LS mean change, % (95% CI), g/L | 19.5 | (5.664 to 33.336) | 29.1 | (16.066 to 42.198) |

| | |
|---|---|
| LS mean difference, % (95% CI), | −9.6 (−25.17 |
| nominal P value | to 5.91), 0.223 |

BICLA, British Isles Lupus Assessment Group-based Composite Lupus Assessment; CI, confidence interval; dsDNA, double-stranded DNA; max, maximum; min, minimum; SD, standard deviation.
^{a}Anti-dsDNA antibody ‘positive’ or ‘negative’ defined as a result of >15 U/mL or ≤15 U/mL, respectively.
^{b}Complement C3 ‘abnormal’ or ‘normal’ levels defined as a result of <0.9 g/L or C3, <0.9 g/L, respectively.
^{c}Complement C4 ‘abnormal’ or ‘normal’ levels defined as a result of <0.1 g/L or C3, <0.1 g/L, respectively.

Only patients with baseline positive anti-dsDNA or abnormal complement C3 or C4 are included in the analysis. Percentage change, difference, CI, and nominal P values calculated using a repeated measures model with fixed effects for baseline value, group, visit, study, and stratification factors. A visit-by-group interaction term was used to account for different relationships across groups and visit was a repeated variable in the model. Percentages do not equal 100% owing to missing data.

Similar proportions of BICLA responders and nonresponders had abnormal C3 and C4 levels at baseline. Percentage changes from baseline to Week 52 in complement levels did not differ between BICLA responders versus nonresponders for C3 (LS mean difference 2.82, 95% CI −4.185 to 9.819, nominal P=0.429) or C4 (LS mean difference −9.63, 95% CI −25.174 to 5.910, nominal P=0.223) (Table 13). More BICLA responders than nonresponders had improvements from abnormal to normal C3 (10.4% vs 7.0%) and C4 (7.5% vs 4.8%).

### 18.3.10.1 Safety

AE frequencies were similar between BICLA responders and nonresponders (83.6% and 85.2%) (Table 18-7). Mild and moderate AEs were reported by similar percentages of BICLA responders and nonresponders, whereas fewer BICLA responders than nonresponders experienced severe AEs (3.8% vs 9.4%). There were no AEs leading to discontinuation (DAE) in BICLA responders compared with 8.2% DAEs in nonresponders. Fewer BICLA responders than nonresponders experienced serious AEs (5.0% vs 19.0%). Fewer BICLA responders than nonresponders had non-opportunistic serious infections (2.2% vs 6.8%). The percentage of patients with herpes zoster was similar in BICLA responders and nonresponders (4.7% vs 3.6%), as was the percentage of patients with influenza (1.9% vs 2.0%) or malignancy (0.6% vs 1.0%).

TABLE 18-7
AEs during treatment in BICLA responders and nonresponders

| | BICLA | | BICLA | |
|---|---|---|---|---|
| | responders | | nonresponders | |
| | at Week 52 | | at Week 52 | |
| AEs, n (%) | (n = 318) | | (n = 501) | |
| Patients with any AE | 266 | (83.6) | 427 | (85.2) |
| Any AE with outcome of | 0 | | 2 | (0.4) |
| death | | | | |
| Any SAE (including events | 16 | (5.0) | 95 | (19.0) |
| with outcome of death) | | | | |
| Lupus-related SAEs | 1 | (0.3) | 15 | (0.3) |
| Lupus nephritis | 0 | | 1 | (0.2) |
| SLE | 1 | (0.3) | 14 | (2.8) |
| Any DAE | 0 | | 41 | (8.2) |
| Any AE related to | 101 | (31.8) | 160 | (31.9) |
| investigational product | | | | |
| Any AE by maximum reported | | | | |
| intensity | | | | |
| Mild | 126 | (39.6) | 183 | (36.5) |
| Moderate | 128 | (40.3) | 197 | (39.3) |
| Severe | 12 | (3.8) | 47 | (9.4) |
| Any AESI | 27 | (8.5) | 67 | (13.4) |
| Non-opportunistic serious | 7 | (2.2) | 34 | (6.8) |
| infection | | | | |
| Opportunistic infections | 0 | | 1 | (0.2) |
| Anaphylaxis | 0 | | 1 | (0.2) |
| Malignancy | 2 | (0.6) | 5 | (1.0) |
| Herpes zoster | 15 | (4.7) | 18 | (3.6) |
| Tuberculosis (including | 2 | (0.6) | 1 | (0.2) |
| latent TB) | | | | |
| Influenza | 6 | (1.9) | 10 | (2.0) |
| Non-SLE vasculitis | 0 | | 0 | |
| Major adverse cardiovascular | 1 | (0.3) | 1 | (0.2) |
| event | | | | |

AE, adverse event; AESI, adverse event of special interest; DAE, adverse event leading to discontinuation; SAE, serious adverse event; SLE, systemic lupus erythematosus; TB, tuberculosis.

### 18.4 Conclusions

BICLA is a dichotomous SLE outcome measure that classifies a patient as a responder or nonresponder based on changes in organ domain activity. As BICLA is primarily used in the clinical trial setting, the aim of this study was to assess the meaningfulness of BICLA response in terms of outcomes that are relevant to patients and physicians. In this post hoc analysis of pooled data acquired from 819 patients enrolled in the TULIP-1 and TULIP-2 trials, BICLA response was significantly associated with improved clinical outcomes across a range of SLE assessments, key PROs, and medical resource utilization measures.

Flares, with or without an increase in glucocorticoid dose, pose significant risks to patients with SLE. In the long term, both disease flares and oral glucocorticoid use have been linked to organ damage, which itself increases mortality risk. Flares also associate with reduced health-related quality of life, and flare severity and oral glucocorticoid use correlates with health care costs. A key SLE treatment goal therefore is to prevent flares while minimizing oral glucocorticoid exposure, which in turn is expected to reduce medical resource utilization. We observed that BICLA responders had fewer disease flares together with a lower daily oral glucocorticoid dose. A greater percentage of BICLA responders achieved sustained oral glucocorticoid reduction to target dose. They also had fewer hospitalizations and ED visits than did nonresponders, including those related to increased SLE activity. Greater improvements in global and tissue-specific disease activity were also observed in responders versus nonresponders, as measured by PGA, SLEDAI-2K, CLASI-A, and joint counts. As improved patient outcomes in disease activity and oral glucocorticoid exposure have been shown to associate with reduced health care costs, BICLA responders may incur lower health care costs than nonresponders.

We also assessed adverse events in BICLA responders and nonresponders. Consistent with the lower flare rates, reduced medical resource utilization, and fewer SLE-related ED visits and hospitalizations associated with BICLA response, there were fewer SAEs in BICLA responders versus nonresponders. While discontinuation of investigational product for any reason led to a patient being classified as a BICLA nonresponder by definition, of note, BICLA nonresponders had a greater propensity to discontinue due to an AE than BICLA responders.

PROs have been incorporated into nearly all SLE clinical trials. However, analyses have often yielded discordance between clinical outcomes and PROs, as patient perceptions of disease activity and illness are heavily impacted by fatigue and quality of life and are not captured by the results of formal measures of disease activity. In the TULIP trials, BICLA responders had improvements in validated PROs, including the physical and mental components of the SF-36 health survey and the FACIT assessment of fatigue. Fatigue, a common symptom in patients with SLE, interferes with daily life, and more than one half of patients with BICLA responses experienced improvement in fatigue in the TULIP trials. PtGA and PGA scores showed concordance in improvement, and greater degrees of improvement among BICLA responders than nonresponders (Table 18-8). Our results demonstrate that BICLA response translates to general improvements in the physical and mental wellbeing of patients with SLE.

Investigation of correlations of SRI(4) response to clinical outcomes in pooled data from 2 phase 2b trials (sifalimumab and anifrolumab), as well as 2 phase 3 trials of belimumab, also demonstrated improved clinical outcomes in SRI(4) responders compared with nonresponders. Whereas changes in serologic outcomes were not significantly different between BICLA responders and nonresponders in the TULIP trials, SRI(4) response was associated with significant improvements in anti-dsDNA antibody and complement C3 levels (but not C4 levels) in the belimumab phase 3 trials. This discordance may be a reflection of the different mechanisms of action of the 2 evaluated drugs, and/or because the BILAG-2004, which measures improvement in BICLA, does not include serology in its scoring system.

The data confirm the value of BICLA as a clinical trial endpoint and that a BICLA response correlates with improvements in a range of other outcomes that resonate with the priorities of both clinicians and patients in everyday practice.

TABLE 18-8
PRO scores at baseline in BICLA responders and nonresponders

| | | |
|---|---|---|
| | BICLA | BICLA |
| | responders | nonresponders |
| PRO | (n = 318) | (n = 501) |

FACIT-F

| | | |
|---|---|---|
| n | 311 | 467 |

| | | | | |
|---|---|---|---|---|
| Mean (SD) | 26.3 | (12.63) | 25.3 | (11.99) |

SF-36 PCS

| | | |
|---|---|---|
| n | 312 | 469 |

| | | | | |
|---|---|---|---|---|
| Mean (SD) | 38.3 | (9.20) | 37.0 | (9.25) |

SF-36 MCS

| | | |
|---|---|---|
| n | 312 | 469 |

| | | | | |
|---|---|---|---|---|
| Mean (SD) | 43.8 | (11.35) | 44.0 | (11.24) |

PtGA

| | | |
|---|---|---|
| n | 311 | 467 |

| | | | | |
|---|---|---|---|---|
| Mean (SD) | 54.7 | (23.36) | 55.8 | (21.44) |

BICLA, British Isles Lupus Assessment Group-based Composite Lupus Assessment; FACIT-F Functional Assessment of Chronic Illness Therapy-Fatigue; MCS, mental component score; PCS, physical component score; PRO, patient-reported outcome; PtGA, Patient's Global Assessment; SD, standard deviation; SF-36-v2, Short Form 36 Health Survey version 2 (acute recall).

### 19 Example 12: Disease Activity in Patients With SLE Coming Off Anifrolumab During the 12-Week Follow-up Period of the Phase 2b MUSE Trial

### 19.1 Introduction

In the randomized, double-blind, phase 2b MUSE trial, anifrolumab treatment reduced disease activity vs placebo across multiple endpoints in patients with moderately to severely active SLE. The inventors assessed for the first time the safety and efficacy in patients coming off anifrolumab during the 12-week (wk) follow-up period in MUSE.

### 19.2 Methods

Patients were randomized 1:1:1 to receive placebo or anifrolumab 300 or 1000 mg every 4 wks; final study dose was Wk 48 and key efficacy endpoints were assessed at Wk 52. Patients were required to complete a 12-wk follow-up period and visits were conducted every 4 wks (±7 days) after the final study dose (FIG. 1). Disease activity was measured using SLEDAI-2K and BILAG-2004. Flares were defined as either ≥1 new BILAG-2004 A or ≥2 new BILAG-2004 B items. Adverse events (AEs) and changes in the 21-gene type I IFN gene signature (IFNGS) were also assessed. All efficacy and IFNGS measures were assessed from Wk 52 to end of follow-up (Wk 60); safety was assessed for 12 wks after the final study dose at Wk 48 or upon study discontinuation. The 21-gene type I IFN gene signature (IFNGS) was assessed over 8 weeks through to 60 weeks. Safety (adverse [AEs]) was evaluated over 12 weeks from Week 48 though to Week 60 or upon study discontinuation.

### 19.3 Results

Of 305 patients randomized in MUSE, 229 completed the last study visit (Wk 52): 86, 75, and 68 from the anifrolumab 300-mg, 1000-mg, and placebo groups, respectively. From Wk 52 to Wk 60, IFNGS expression increased more rapidly in the anifrolumab 300-mg group (mean neutralization ratio: 55.6% to −81.8%) vs the 1000-mg group (71.7% to 31.9%), with negligible changes in the placebo group (−59.2% to −62.6%). From Wk 52 to the end of the follow-up period (Wk 60), mean global SLEDAI-2K scores increased in patients coming off anifrolumab 300 mg (4.3 to 5.0 [mean change: 0.7]) and 1000 mg (3.8 to 4.1 [0.3]) but not for the placebo group (5.9 to 5.8 [−0.1]). A similar trend was observed in mean global BILAG-2004 scores in patients coming off anifrolumab 300 mg (6.0 to 8.5 [2.4]) vs placebo (8.3 to 9.1 [0.8]).

Mucocutaneous was the most frequent organ system associated with worsening in patients ceasing anifrolumab, with shifts in the percentages of patients with BILAG C/D/E scores to BILAG A/B scores; similar trends were also observed in the musculoskeletal organ system. Worsening was most frequent in the mucocutaneous domain in patients coming off anifrolumab, with shifts in the percentages of patients with BILAG-2004 C/D/E to A/B scores (FIG. 58C); similar trends were also observed in the musculoskeletal domain. Overall, 15.2% and 6.7% of patients coming off anifrolumab 300 or 1000 mg, respectively, had ≥1 flare in the follow-up period vs 2.0% with placebo.

Mean Cutaneous Lupus Erythematosus Disease Area and Severity Index (CLASI) scores increased slightly from Wk 52 to Wk 60 across the anifrolumab 300-mg, 1000-mg, and placebo groups (from 1.9 to 2.4, 1.8 to 2.2, and 3.5 to 4.0, respectively) (FIG. 59).

From Wk 52 to Wk 60, IFNGS expression increased more rapidly in the anifrolumab 300-mg group (mean neutralization ratio: 55.6% to −81.8%) vs the 1000-mg group (71.7% to 31.9%), with negligible changes in the placebo group (−59.2% to −62.6%).

AEs during the 12-wk follow-up period were similar between the anifrolumab 300-mg and 1000-mg vs placebo groups (≥1 AE: 29.3% and 26.7% vs 24.8%; ≥1 serious AE: 3.0% and 3.8% vs 5.0%). Disease activity, measured using MDGA score, increased between Week 52 and Week 60 in both anifrolumab 300-mg and 1000-mg groups; there was no change in the placebo group. Active joint counts increased slightly from Week 52 to Week 60 across the anifrolumab 300-mg, anifrolumab 1000-mg, and placebo groups (FIG. 59A). Overall, more patients ceasing treatment of anifrolumab 300 or 1000 mg had ≥1 BILAG flare from Week 52 through Week 60 versus placebo (FIG. 59B).

### 19.4 Conclusion

There was a notable trend toward worsening in disease activity in patients coming off anifrolumab vs placebo using SLEDAI-2K and BILAG-2004. This was associated with a rebound in IFNGS in patients previously treated with anifrolumab, an effect more apparent with 300 vs 1000 mg.

### 20 REFERENCES

(1) Bruce, I. N.; O'Keeffe, A. G.; Farewell, V.; Hanly, J. G.; Manzi, S.; Su, L.; Gladman, D. D.; Bae, S.-C.; Sanchez-Guerrero, J.; Romero-Diaz, J.; Gordon, C.; Wallace, D. J.; Clarke, A. E.; Bernatsky, S.; Ginzler, E. M.; Isenberg, D. A.; Rahman, A.; Merrill, J. T.; Alarcón, G. S.; Fessler, B. J.; Fortin, P. R.; Petri, M.; Steinsson, K.; Dooley, M. A.; Khamashta, M. A.; Ramsey-Goldman, R.; Zoma, A. A.; Sturfelt, G. K.; Nived, O.; Aranow, C.; Mackay, M.; Ramos-Casals, M.; van Vollenhoven, R. F.; Kalunian, K. C.; Ruiz-Irastorza, G.; Lim, S.; Kamen, D. L.; Peschken, C. A.; Inanc, M.; Urowitz, M. B. Factors Associated with Damage Accrual in Patients with Systemic Lupus Erythematosus: Results from the Systemic Lupus International Collaborating Clinics (SLICC) Inception Cohort. Ann Rheum Dis 2015, 74 (9), 1706-1713. https://doi.org/10.1136/annrheumdis-2013-205171.
(2) Petri, M. Long-Term Outcomes in Lupus. Am J Manag Care 2001, 7 (16 Suppl), S480-485.
(3) Arriens, C.; Wren, J. D.; Munroe, M. E.; Mohan, C. Systemic Lupus Erythematosus Biomarkers: The Challenging Quest. Rheumatology (Oxford) 2017, 56 (Suppl 1), i32-i45. https://doi.org/10.1093/rheumatology/kew407.
(4) Rousset, F.; Garcia, E.; Defrance, T.; Péronne, C.; Vezzio, N.; Hsu, D. H.; Kastelein, R.; Moore, K. W.; Banchereau, J. Interleukin 10 Is a Potent Growth and Differentiation Factor for Activated Human B Lymphocytes. Proc Natl Acad Sci USA 1992, 89 (5), 1890-1893.
(5) Llorente, L.; Richaud-Patin, Y.; García-Padilla, C.; Claret, E.; Jakez-Ocampo, J.; Cardiel, M. H.; Alcocer-Varela, J.; Grangeot-Keros, L.; Alarcón-Segovia, D.; Wijdenes, J.; Galanaud, P.; Emilie, D. Clinical and Biologic Effects of Anti-Interleukin-10 Monoclonal Antibody Administration in Systemic Lupus Erythematosus. Arthritis and Rheumatism 2000, 43 (8), 1790-1800. https://doi.org/10.1002/1529-0131 (200008)43:8<1790::AID-ANR15>3.0.CO; 2-2.
(6) Yao, Y.; Higgs, B. W.; Morehouse, C.; de Los Reyes, M.; Trigona, W.; Brohawn, P.; White, W.; Zhang, J.; White, B.; Coyle, A. J.; Kiener, P. A.; Jallal, B. Development of Potential Pharmacodynamic and Diagnostic Markers for Anti-IFN-α Monoclonal Antibody Trials in Systemic Lupus Erythematosus. Hum Genomics Proteomics 2009, 2009. https://doi.org/10.4061/2009/374312.
(7) Hrovat, A.; Zavec, A. B.; Pogačnik, A.; Frangež, R.; Vrecl, M. Establishing and Functional Characterization of an HEK-293 Cell Line Expressing Autofluorescently Tagged β-Actin (PEYFP-ACTIN) and the Neurokinin Type 1 Receptor (NK1-R). Cell Mol Biol Lett 2009, 15 (1), 55. https://doi.org/10.2478/s11658-009-0034-0.
(8) Švec, J.; Ergang, P.; Mandys, V.; Kment, M.; Pácha, J. Expression Profiles of Proliferative and Antiapoptotic Genes in Sporadic and Colitis-Related Mouse Colon Cancer Models. Int J Exp Pathol 2010, 91 (1), 44-53. https://doi.org/10.1111/j.1365-2613.2009.00698.x.
(9) Psarras, A.; Emery, P.; Vital, E. M. Type I Interferon-Mediated Autoimmune Diseases: Pathogenesis, Diagnosis and Targeted Therapy. Rheumatology (Oxford) 2017, 56 (10), 1662-1675. https://doi.org/10.1093/rheumatology/kew431.
(10) Khamashta, M.; Merrill, J. T.; Werth, V. P.; Furie, R.; Kalunian, K.; Illei, G. G.; Drappa, J.; Wang, L.; Greth, W. Sifalimumab, an Anti-Interferon-α Monoclonal Antibody, in Moderate to Severe Systemic Lupus Erythematosus: A Randomised, Double-Blind, Placebo-Controlled Study. Ann Rheum Dis 2016, 75 (11), 1909-1916. https://doi.org/10.1136/annrheumdis-2015-208562.
(11) Higgs, B. W.; Zhu, W.; Morehouse, C.; White, W. I.; Brohawn, P.; Guo, X.; Rebelatto, M.; Le, C.; Amato, A.; Fiorentino, D.; Greenberg, S. A.; Drappa, J.; Richman, L.; Greth, W.; Jallal, B.; Yao, Y. A Phase 1b Clinical Trial Evaluating Sifalimumab, an Anti-IFN-α Monoclonal Antibody, Shows Target Neutralisation of a Type I IFN Signature in Blood of Dermatomyositis and Polymyositis Patients. Ann Rheum Dis 2014, 73 (1), 256-262. https://doi.org/10.1136/annrheumdis-2012-202794.
(12) Ramaswamy, M.; Tummala, R.; Streicher, K.; Nogueira da Costa, A.; Brohawn, P. Z. The Pathogenesis, Molecular Mechanisms, and Therapeutic Potential of the Interferon Pathway in Systemic Lupus Erythematosus and Other Autoimmune Diseases. Int J Mol Sci 2021, 22 (20), 11286. https://doi.org/10.3390/ijms222011286.
(13) Vilas-Boas, A.; Morais, S. A.; Isenberg, D. A. Belimumab in Systemic Lupus Erythematosus. RMD Open 2015, 1 (1). https://doi.org/10.1136/rmdopen-2014-000011.
(14) Target Modulation of a Type I Interferon Gene Signature and Pharmacokinetics of Anifrolumab in a Phase IIb Study of Patients with Moderate to Severe Systemic Lupus Erythematosus. ACR Meeting Abstracts.
(15) van Vollenhoven, R. F.; Stohl, W.; Furie, R. A.; Fox, N. L.; Groark, J. G.; Bass, D.; Kurtinecz, M.; Pobiner, B. F.; Eastman, W. J.; Gonzalez-Rivera, T.; Gordon, D. Clinical Response beyond the Systemic Lupus Erythematosus Responder Index: Post-Hoc Analysis of the BLISS-SC Study. Lupus Sci Med 2018, 5 (1). https://doi.org/10.1136/lupus-2018-000288.

1-10. (canceled)

11. A method of treating SLE in a subject in need thereof, the method comprising administering a therapeutically effective amount of anifrolumab, wherein the subject has an IL-10 plasma concentration lower than a about 2 pg/ml at baseline, wherein the treatment reduces SLE disease activity in the subject.

12. The method of claim 11, wherein the subject is identified as having an elevated IFNGS compared to a healthy subject.

13. The method of claim 12, wherein an elevated IFNGS comprises at least about four-fold increase in mRNA of at least four of IFI27, IFI44, IFI44L, IFI6, and RSAD2 in a sample from the subject and/or subjects, relative to a sample from a healthy subject.

14. The method of claim 12, wherein an elevated IFNGS comprises at least about four-fold increase in mRNA of at least four of IFI27, IFI44, IFI44L, IFI6, and RSAD2 in a sample from the subject and/or subjects, relative to pooled samples from healthy patients.

15. The method of claim 13, wherein the mRNA is increased relative to the mRNA of one or more control genes present in the sample.

16. The method of claim 15, wherein the one or more control genes are chosen from ACTB, GAPDH, and 18S rRNA.

17-23. (canceled)

24. The method of claim 11, comprising determining the IL-10 concentration in a sample from the patient, optionally wherein the sample is isolated from the subject.

25. The method of claim 24, wherein the method comprises measuring the IL-10 concentration in the sample from the patient.

26. The method of claim 25, wherein the IL-10 concentration is measured by immunoassay.

27. The method of claim 26, wherein the immunoassay is a Luminex or Simoa immunoassay.

28. The method of claim 24, wherein the sample is a blood, serum or plasma sample.

29-74. (canceled)

75. The method of claim 11, wherein the treatment comprises administering 300 mg anifrolumab.

76. The method of claim 75, wherein anifrolumab is administered as an intravenous (IV) infusion.

77. The method according to claim 75, wherein anifrolumab is administered every four weeks.

78. The method of claim 11, wherein the treatment comprises administering about 120 mg anifrolumab.

79. The method of claim 78, wherein anifrolumab is administered subcutaneously.

80. The method according to claim 78, wherein anifrolumab is administered every week.

81-86. (canceled)

87. A method of treating SLE in a subject in need thereof, the method comprising administering a therapeutically effective amount of an IFNR inhibitor, wherein the IFNR inhibitor comprises:
a) a heavy chain variable region complementarity determining region 1 (HCDR1) comprising the amino acid sequence of SEQ ID NO: 3;
b) a heavy chain variable region complementarity determining region 2 (HCDR2) comprising the amino acid sequence of SEQ ID NO: 4;
c) a heavy chain variable region complementarity determining region 3 (HCDR3) comprising the amino acid sequence of SEQ ID NO: 5;
d) a light chain variable region complementarity determining region 1 (LCDR1) comprising the amino acid sequence SEQ ID NO: 6;
e) a light chain variable region complementarity determining region 2 (LCDR2) comprising the amino acid sequence SEQ ID NO: 7; and
f) a light chain variable region complementarity determining region 3 (LCDR3) comprising the amino acid sequence SEQ ID NO: 8,
wherein the subject has an IL-10 plasma concentration lower than about 2 pg/ml at baseline, wherein the treatment reduces SLE disease activity in the subject.

88. The method of claim 87, wherein the IFNR inhibitor comprises: (a) a human heavy chain variable region comprising the amino acid sequence of SEQ ID NO: 1; and (b) a human light chain variable region comprising the amino acid sequence of SEQ ID NO: 2.

89. The method of claim 87, wherein the IFNR inhibitor comprises an Fc region comprising an amino acid substitution of L234F, as numbered by the EU index as set forth in Kabat, and wherein the antibody exhibits reduced affinity for at least one Fc ligand compared to an unmodified antibody, optionally wherein the antibody comprises in the Fc region an amino acid substitution of L234F, L235E and/or P331S, as numbered by the EU index as set forth in Kabat.


SEQUENCE LISTING (24 sequences)

SEQ ID NO: 1 - protein, 117 aa, Artificial Sequence [sequence omitted]

SEQ ID NO: 2 - protein, 108 aa, Artificial Sequence [sequence omitted]

SEQ ID NO: 3 - protein, 5 aa, Artificial Sequence
  HCDR1
NYWIA

SEQ ID NO: 4 - protein, 17 aa, Artificial Sequence
  HCDR2
IIYPGDSDIRYSPSFQG

SEQ ID NO: 5 - protein, 8 aa, Artificial Sequence
  HCDR3
HDIEGFDY

SEQ ID NO: 6 - protein, 12 aa, Artificial Sequence
  LCDR1
RASQSVSSSFFA

SEQ ID NO: 7 - protein, 7 aa, Artificial Sequence
  LCDR2
GASSRAT

SEQ ID NO: 8 - protein, 9 aa, Artificial Sequence
  LCDR3
QQYDSSAIT

SEQ ID NO: 9 - protein, 107 aa, Artificial Sequence [sequence omitted]

SEQ ID NO: 10 - protein, 330 aa, Artificial Sequence
  Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 447 aa, Artificial Sequence
  Heavy chain
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, Artificial Sequence
  Light chain
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 80 aa, Artificial Sequence
  H15D10 - VH
EVQLVQSGAEVKKPGESLRISCKGSGYTFTNYWVAWVRQMPGKGLESMGIIYPGDSDTRYSPSFQGHVTISADKSISTAY

SEQ ID NO: 14 - protein, 108 aa, Artificial Sequence
  L8C3 - VL
DIQMTQSPSSLSASLGDRVTITCRASQNVGNYLNWYQQKPGKAPKLLIYRASNLASGVPSRFSGSGSGTDFTLTISSLQPEDFATYYCQQMEHAPPTFGQGTKVEIKR

SEQ ID NO: 15 - protein, 108 aa, Artificial Sequence
  L16C11 - VL
EIVLTQSPGTLSLSPGERATLSCRASQSVIGYYLAWYQQKPGQAPRLLIYSVSTLASGIPDRFSGSGSGTDFTLTISRLEPEDFAVYYCQQYYRFPITFGQGTKVEIK

SEQ ID NO: 16 - protein, 117 aa, Artificial Sequence
  H19B7 - VH
EVQLVQSGAEVKKPGESLRISCKGSGYTFTNYWMAWVRQMPGKGLESMGIIYPSDSDTRYSPSFQGHVTISADKSISTAYLQWSSLKASDTAMYYCARHDVEGYDYWGQGTLVTVSS

SEQ ID NO: 17 - protein, 111 aa, Artificial Sequence
  BT-063 VL
DVVMTQSPLSLPVTLGQPASISCRSSQNIVHSNGNTYLEWYLQRPGQSPRLLIYKVSNRFSGVPDRFSGSGSGTDFTLISRVEAEDVGVYYCFQGSHVPWTFGQGTKVEIK

SEQ ID NO: 18 - protein, 117 aa, Artificial Sequence
  VH
EVQLVESGGGLVQPGGSLRLSCAASGFSFATYGVHWVRQSPGKGLEWLGVIWRGGSTDYSAAFMSRLTISKDNSKNTVYLQMNSLRAEDTAVYFCAKQAYGHYMDYWGQGTSVTVSS

SEQ ID NO: 19 - protein, 17 aa, Artificial Sequence
  LCDR1
ISCRSSQNIVHSNGNTY

SEQ ID NO: 20 - protein, 12 aa, Artificial Sequence
  LCDR2
KVSNRFSGVPDR

SEQ ID NO: 21 - protein, 7 aa, Artificial Sequence
  LCDR3
QGSHVPW

SEQ ID NO: 22 - protein, 9 aa, Artificial Sequence
  HCDR1
ASGFSFATY

SEQ ID NO: 23 - protein, 16 aa, Artificial Sequence
  HCDR2
IWRGGSTDYSAAFMSR

SEQ ID NO: 24 - protein, 8 aa, Artificial Sequence
  HCDR3
QAYGHYMD